SEQUENCE LISTING (84 sequences)

SEQ ID NO: 1 - DNA, 6 bp, Artificial Sequence
  Description of Artificial Sequence; note = synthetic construct
agaaca

SEQ ID NO: 2 - DNA, 6 bp, Artificial Sequence
  Description of Artificial Sequence; note = synthetic construct
aggtca

SEQ ID NO: 3 - DNA, 15 bp, Artificial Sequence
  Description of Artificial Sequence; note = synthetic construct
  misc_feature 7,8,9: n = a, g, c or t(u)
agaacannntgttct

SEQ ID NO: 4 - DNA, 13 bp, Artificial Sequence
  Description of Artificial Sequence; note = synthetic construct
  misc_feature 7: n = a, c, g or t(u)
aggtcanaggtca

SEQ ID NO: 5 - DNA, 12 bp, Artificial Sequence
  Description of Artificial Sequence; note = synthetic construct
aggtcatgacct

SEQ ID NO: 6 - DNA, 13 bp, Artificial Sequence
  Description of Artificial Sequence; note = synthetic construct
ggtcacggtggcc

SEQ ID NO: 7 - DNA, 13 bp, Artificial Sequence
  Description of Artificial Sequence; note = synthetic construct
ggtgaccttgacc

SEQ ID NO: 8 - DNA, 13 bp, Artificial Sequence
  Description of Artificial Sequence; note = synthetic construct
ggtcaaggcgatc

SEQ ID NO: 9 - DNA, 13 bp, Artificial Sequence
  Description of Artificial Sequence; note = synthetic construct
  misc_feature 6,7,8: n = a, c, g or t(u)
ggtcannntgacc

SEQ ID NO: 10 - DNA, 13 bp, Artificial Sequence
  Description of Artificial Sequence; note = synthetic construct
gggcatcgtgacc

SEQ ID NO: 11 - DNA, 13 bp, Artificial Sequence
  Description of Artificial Sequence; note = synthetic construct
ggtcgccaggacc

SEQ ID NO: 12 - DNA, 13 bp, Artificial Sequence
  Description of Artificial Sequence; note = synthetic construct
ggtcaggctggtc

SEQ ID NO: 13 - DNA, 13 bp, Artificial Sequence
  Description of Artificial Sequence; note = synthetic construct
ggccgggctgacc

SEQ ID NO: 14 - DNA, 13 bp, Artificial Sequence
  Description of Artificial Sequence; note = synthetic construct
ggccgggctgacc

SEQ ID NO: 15 - DNA, 13 bp, Artificial Sequence
  Description of Artificial Sequence; note = synthetic construct
ggccacgatgaca

SEQ ID NO: 16 - DNA, 13 bp, Artificial Sequence
  Description of Artificial Sequence; note = synthetic construct
ggtggccctgacc

SEQ ID NO: 17 - DNA, 13 bp, Artificial Sequence
  Description of Artificial Sequence; note = synthetic construct
ggtcaaggtgacc

SEQ ID NO: 18 - DNA, 13 bp, Artificial Sequence
  Description of Artificial Sequence; note = synthetic construct
ggtcatggtgacc

SEQ ID NO: 19 - DNA, 13 bp, Artificial Sequence
  Description of Artificial Sequence; note = synthetic construct
gcaggagctgacc

SEQ ID NO: 20 - DNA, 13 bp, Artificial Sequence
  Description of Artificial Sequence; note = synthetic construct
ggtcagcgtggcc

SEQ ID NO: 21 - DNA, 13 bp, Artificial Sequence
  Description of Artificial Sequence; note = synthetic construct
aatcagactgact

SEQ ID NO: 22 - DNA, 13 bp, Artificial Sequence
  Description of Artificial Sequence; note = synthetic construct
ggtcaggctggtc

SEQ ID NO: 23 - protein, 69 aa, Artificial Sequence
  Description of Artificial Sequence; note = synthetic construct
TRYCAVCNDYASGYHYGVWSCEGCKAFFKRSIQGHNDYMCPATNQCTIDKNRRKSCQACRLRKCYEVGM

SEQ ID NO: 24 - protein, 51 aa, Artificial Sequence
  Description of Artificial Sequence; note = synthetic construct
MKGGIRKDRRGGRMLKHKRQRDDGEGRGEVGSAGDMRAANLWPSPLMIKRS

SEQ ID NO: 25 - protein, 70 aa, Artificial Sequence
  Description of Artificial Sequence; note = synthetic construct
DAHFCAVCSDYASGYHYGVWSCEGCKAFFKRSIQGHNDYICPATNQCTIDKNRRKSCQACRLRKCYEVGM

SEQ ID NO: 26 - protein, 29 aa, Artificial Sequence
  Description of Artificial Sequence; note = synthetic construct
VKCGSRRERCGYRLVRRQRSADEQLHCAG

SEQ ID NO: 27 - protein, 74 aa, Artificial Sequence
  Description of Artificial Sequence; note = synthetic construct
PPKLCLVCSDEASGCHYGVLTCGSCKVFFKRAVEGQHNYLCAGRNDCIIDKIRRKNCPACRYRKCLQAGMNLEA

SEQ ID NO: 28 - protein, 43 aa, Artificial Sequence
  Description of Artificial Sequence; note = synthetic construct
RKTKKKIKGIQQATTGVSQETSENPGNKTIVPATLPQLTPTGR

SEQ ID NO: 29 - protein, 72 aa, Artificial Sequence
  Description of Artificial Sequence; note = synthetic construct
PQKTCLICGDEASGCHYGALTCGSCKVFFKRAAEGKQKYLCASRNDCTIDKFRRKNCPSCRLRKCYEAGMTL

SEQ ID NO: 30 - protein, 56 aa, Artificial Sequence
  Description of Artificial Sequence; note = synthetic construct
GARKLKKLGNLKLQEEGEASSTTSPTEETTQKLTVSHIEGYECQPIFLNVLEAIAR

SEQ ID NO: 31 - protein, 72 aa, Artificial Sequence
  Description of Artificial Sequence; note = synthetic construct
PQKICLICGDEASGCHYGVLTCGSCKVFFKRAMEGQHNYLCAGRNDCIVDKIRRKNCPACRLRKCCQAGMVL

SEQ ID NO: 32 - protein, 53 aa, Artificial Sequence
  Description of Artificial Sequence; note = synthetic construct
GGRKFKKFNKVRVVRALDAVALPQPVGVPNESQALSQRFTFSPGQDIQLIPPL

SEQ ID NO: 33 - protein, 72 aa, Artificial Sequence
  Description of Artificial Sequence; note = synthetic construct
KDELCVVCGDKATGYHYRCITCEGCKGFFRRTIQKNLHPSYSCKYEGKCVIDKVTRNQCQECRFKKCIYVGM

SEQ ID NO: 34 - protein, 63 aa, Artificial Sequence
  Description of Artificial Sequence; note = synthetic construct
ATDLVLDDSKRLAKRKLIEENREKRRREELQKSIGHKPEPTDEEWELIKTVTEAHVATNAQGS

SEQ ID NO: 35 - protein, 70 aa, Artificial Sequence
  Description of Artificial Sequence; note = synthetic construct
TKHICAICGDRSSGKHYGVYSCEGCKGFFKRTVRKDLTYTCRDNKDCLIDKRQRNRCQYCRYQKCLAMGM

SEQ ID NO: 36 - protein, 59 aa, Artificial Sequence
  Description of Artificial Sequence; note = synthetic construct
KREAVQEERQRGKDRNENEVESTSSANEDMPVERILEAELAVEPKTETYVEANMGLNPS

SEQ ID NO: 37 - protein, 72 aa, Artificial Sequence
  Description of Artificial Sequence; note = synthetic construct
IYKPCFVCQDKSSGYHYGVSACEGCKGFFRRSIQKNMVYTCHRDKNCIINKVTRNRCQYCRLQKCFEVGMSK

SEQ ID NO: 38 - protein, 69 aa, Artificial Sequence
  Description of Artificial Sequence; note = synthetic construct
ESVRNDRNKKKKEVPKPECSESYTLTPEVGELIEKVRKAHQETFPALCQLGKYTTNNSSEQRVSLDIDL

SEQ ID NO: 39 - protein, 70 aa, Artificial Sequence
  Description of Artificial Sequence; note = synthetic construct
RICGVCGDRATGFHFNAMTCEGCKGFFRRSMKRKALFTCPFNGDCRITKDNRRHCQACRLKRCVDIGMMK

SEQ ID NO: 40 - protein, 65 aa, Artificial Sequence
  Description of Artificial Sequence; note = synthetic construct
EFILTDEEVQRKREMILKRKEEEALKDSLRPKLSEEQQRIIAILLDAHHKTYDPTYSDFCQFRPP

SEQ ID NO: 41 - protein, 134 aa, Artificial Sequence
  Description of Artificial Sequence; note = synthetic construct
PPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQIS

SEQ ID NO: 42 - protein, 87 aa, Artificial Sequence
  Description of Artificial Sequence; note = synthetic construct
KVAPPTDVSLGDELHLDGEDVAMAHADALDDFDLDMLGDGDSPGPGFTPHDSAPYGALDMADFEFEQMFTDALGIDEYGGEFPGIRR

SEQ ID NO: 43 - protein, 35 aa, Artificial Sequence
  Description of Artificial Sequence; note = synthetic construct
MAAAVRMNIQMLLEAADYLERREREAEHGYASMLP

SEQ ID NO: 44 - protein, 90 aa, Artificial Sequence
  Description of Artificial Sequence; note = synthetic construct
MDAKSLTAWSRTLVTFKDVFVDFTREEWKLLDTAQQIVYRNVMLENYKNLVSLGYQLTKPDVILRLEKGEEPWLVEREIHQETHPDSETA

SEQ ID NO: 45 - protein, 595 aa, Artificial Sequence
  Description of Artificial Sequence; note = synthetic construct
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 46 - DNA, 2092 bp, Artificial Sequence
  Description of Artificial Sequence; note = synthetic construct
gaattccaaaattgtgatgtttcttgtatttttgatgaaggagaaatactgtaatgatcactgtttacactatgtacactttaggccagccctttgtagcgttatacaaactgaaagcacaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggc

SEQ ID NO: 47 - DNA, 207 bp, Artificial Sequence
  Description of Artificial Sequence; note = synthetic construct
actcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatg

SEQ ID NO: 48 - DNA, 153 bp, Artificial Sequence
  Description of Artificial Sequence; note = synthetic construct
atgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctct

SEQ ID NO: 49 - protein, 530 aa, Artificial Sequence
  Description of Artificial Sequence; note = synthetic construct
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

SEQ ID NO: 50 - DNA, 2011 bp, Artificial Sequence
  Description of Artificial Sequence; note = synthetic construct
tttcagtttctccagctgctggctttttggacacccactcccccgccaggaggcagttgcaagcgcggaggctgcgagaaataactgcctcttgaaacttgcagggcgaagagcaggcggcgagcgctgggccggggagggaccacccgagctgcgacgggctctggggctgcggggcagggctggcgcccggagcctgagctgcaggaggtgcgctcgctttcctcaacaggtggcggcggggcgcgcgccgggagaccccccctaatgcgggaaaagcacgtgtccgcattttagagaaggcaaggccggtgtgtttatctgcaagccattatacttgcccacgaatctttgagaacattataatgacctttgtgcctcttcttgcaaggtgttttctcagctgttatctcaagacatggatataaaaaactcaccatctagccttaattctccttcctcctacaactgcagtcaatccatcttacccctggagcacggctccatatacataccttcctcctatgtagacagccaccatgaatatccagccatgacattctatagccctgctgtgatgaattacagcattcccagcaatgtcactaacttggaaggtgggcctggtcggcagaccacaagcccaaatgtgttgtggccaacacctgggcacctttctcctttagtggtccatcgccagttatcacatctgtatgcggaacctcaaaagagtccctggtgtgaagcaagatcgctagaacacaccttacctgtaaacagagagacactgaaaaggaaggttagtgggaaccgttgcgccagccctgttactggtccaggttcaaagagggatgctcacttctgcgctgtctgcagcgattacgcatcgggatatcactatggagtctggtcgtgtgaaggatgtaaggccttttttaaaagaagcattcaaggacataatgattatatttgtccagctacaaatcagtgtacaatcgataaaaaccggcgcaagagctgccaggcctgccgacttcggaagtgttacgaagtgggaatggtgaagtgtggctcccggagagagagatgtgggtaccgccttgtgcggagacagagaagtgccgacgagcagctgcactgtgccggcaaggccaagagaagtggcggccacgcgccccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagtaacaagggcatggaacatctgctcaacatgaagtgcaaaaatgtggtcccagtgtatgacctgctgctggagatgctgaatgcccacgtgcttcgcgggtgcaagtcctccatcacggggtccgagtgcagcccggcagaggacagtaaaagcaaagagggctcccagaacccacagtctcagtga

SEQ ID NO: 51 - DNA, 210 bp, Artificial Sequence
  Description of Artificial Sequence; note = synthetic construct
gatgctcacttctgcgctgtctgcagcgattacgcatcgggatatcactatggagtctggtcgtgtgaaggatgtaaggccttttttaaaagaagcattcaaggacataatgattatatttgtccagctacaaatcagtgtacaatcgataaaaaccggcgcaagagctgccaggcctgccgacttcggaagtgttacgaagtgggaatg

SEQ ID NO: 52 - DNA, 87 bp, Artificial Sequence
  Description of Artificial Sequence; note = synthetic construct
gtgaagtgtggctcccggagagagagatgtgggtaccgccttgtgcggagacagagaagtgccgacgagcagctgcactgtgccggc

SEQ ID NO: 53 - protein, 777 aa, Artificial Sequence
  Description of Artificial Sequence; note = synthetic construct
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 54 - DNA, 4788 bp, Artificial Sequence
  Description of Artificial Sequence; note = synthetic construct
tttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttg

SEQ ID NO: 55 - DNA, 222 bp, Artificial Sequence
  Description of Artificial Sequence; note = synthetic construct
cctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagct

SEQ ID NO: 56 - DNA, 123 bp, Artificial Sequence
  Description of Artificial Sequence; note = synthetic construct
cgaaaaacaaagaaaaaaataaaaggaattcagcaggccactacaggagtctcacaagaaacctctgaaaatcctggtaacaaaacaatagttcctgcaacgttaccacaactcacccctacc

SEQ ID NO: 57 - protein, 734 aa, Artificial Sequence
  Description of Artificial Sequence; note = synthetic construct
MQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGGGGEAEAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 58 - DNA, 2205 bp, Artificial Sequence
  Description of Artificial Sequence; note = synthetic construct
atgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggtggcggcggcggcggcggcggcggcggcggcggcggcggcggcggcggcggcggcggcggcgaggcggaagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtga

SEQ ID NO: 59 - DNA, 216 bp, Artificial Sequence
  Description of Artificial Sequence; note = synthetic construct
ccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctg

SEQ ID NO: 60 - DNA, 162 bp, Artificial Sequence
  Description of Artificial Sequence; note = synthetic construct
ggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccatt

SEQ ID NO: 61 - protein, 933 aa, Artificial Sequence
  Description of Artificial Sequence; note = synthetic construct
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGRKFKKFNKVRVVRALDAVALPQPVGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 62 - DNA, 3014 bp, Artificial Sequence
  Description of Artificial Sequence; note = synthetic construct
ctgaccagcgccgccctcccccgcccccgacccaggaggtggagatccctccggtccagccacattcaacacccactttctcctccctctgcccctatattcccgaaaccccctcctccttcccttttccctcctccctggagacgggggaggagaaaaggggagtccagtcgtcatgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtgg

SEQ ID NO: 63 - DNA, 216 bp, Artificial Sequence
  Description of Artificial Sequence; note = synthetic construct
cctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtcctt

SEQ ID NO: 64 - DNA, 160 bp, Artificial Sequence
  Description of Artificial Sequence; note = synthetic construct
ggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactga

SEQ ID NO: 65 - protein, 455 aa, Artificial Sequence
  Description of Artificial Sequence; note = synthetic construct
MTENGLTAWDKPKHCPDREHDWKLVGMSEACLHRKSHSERRSTLKNEQSSPHLIQTTWTSSIFHLDHDDVNDQSVSSAQTFQTEEKKCKGYIPSYLDKDELCVVCGDKATGYHYRCITCEGCKGFFRRTIQKNLHPSYSCKYEGKCVIDKVTRNQCQECRFKKCIYVGMATDLVLDDSKRLAKRKLIEENREKRRREELQKSIGHKPEPTDEEWELIKTVTEAHVATNAQGSHWKQKPKFLPEDIGQAPIVNAPEGGKVDLEAFSHFTKIITPAITRVVDFAKKLPMFCELPCEDQIILLKGCCMEIMSLRAAVRYDPESETLTLNGEMAVIRGQLKNGGLGVVSDAIFDLGMSLSSFNLDDTEVALLQAVLLMSSDRPGLACVERIEKYQDSFLLAFEHYINYRKHHVTHFWPKLLMKVTDLRMIGACHASRFLHMKVECPTELLPPLFEVFED

SEQ ID NO: 66 - DNA, 1698 bp, Artificial Sequence
  Description of Artificial Sequence; note = synthetic construct
cggcggggatcaactttgcatgaataatgtgagtgcgcttggaaaagagacctcctgctccgcgggctcggggcaagagcccgcaggctaccttccccgggcaggggcgctcaacccaaccggctccagggcactggtaatttggctagaggaccgcgcggaggcagcgggatctgcgatttccttctggttggctgtcctgcgtgggtgccaagttccacacatgatttaatgaataagaaggagatgtcagtgaaaaaagggatccagaatgattactaacctataacccccaacagtatgacagaaaatggccttacagcttgggacaaaccgaagcactgtccagaccgagaacacgactggaagctagtaggaatgtctgaagcctgcctacataggaagagccattcagagaggcgcagcacgttgaaaaatgaacagtcgtcgccacatctcatccagaccacttggactagctcaatattccatctggaccatgatgatgtgaacgaccagagtgtctcaagtgcccagaccttccaaacggaggagaagaaatgtaaagggtacatccccagttacttagacaaggacgagctctgtgtagtgtgtggtgacaaagccaccgggtatcactaccgctgtatcacgtgtgaaggctgcaagggtttctttagaagaaccattcagaaaaatctccatccatcctattcctgtaaatatgaaggaaaatgtgtcatagacaaagtcacgcgaaatcagtgccaggaatgtcgctttaagaaatgcatctatgttggcatggcaacagatttggtgctggatgacagcaagaggctggccaagaggaagctgatagaggagaaccgggagaaaagacggcgggaagagctgcagaagtccatcgggcacaagccagagcccacagacgaggaatgggagctcatcaaaactgtcaccgaagcccatgtggcgaccaacgcccaaggcagccactggaagcaaaaaccgaaatttctgccagaagacattggacaagcaccaatagtcaatgccccagaaggtggaaaggttgacttggaagccttcagccattttacaaaaatcatcacaccagcaattaccagagtggtggattttgccaaaaagttgcctatgttttgtgagctgccatgtgaagaccagatcatcctcctcaaaggctgctgcatggagatcatgtcccttcgcgctgctgtgcgctatgacccggaaagtgagactttaaccttgaatggggaaatggcagtgatacggggccagctgaaaaatgggggtcttggggtggtgtcagacgccatctttgacctaggcatgtctctgtcttctttcaacctggatgacactgaagtagccctccttcaggccgtcctgctgatgtcttcagatcgcccggggcttgcctgtgttgagagaatagaaaagtaccaagatagtttcctgctggcctttgaacactatatcaattaccgaaaacaccacgtgacacacttttggccaaaactcctgatgaaggtgacagatctgcggatgataggagcctgccatgccagccgcttcctgcacatgaaggtggaatgccccacagaactcctcccccctttgttcctggaagtgttcgaggattagactgactggattccttcctataattcc

SEQ ID NO: 67 - DNA, 216 bp, Artificial Sequence
  Description of Artificial Sequence; note = synthetic construct
aaggacgagctctgtgtagtgtgtggtgacaaagccaccgggtatcactaccgctgtatcacgtgtgaaggctgcaagggtttctttagaagaaccattcagaaaaatctccatccatcctattcctgtaaatatgaaggaaaatgtgtcatagacaaagtcacgcgaaatcagtgccaggaatgtcgctttaagaaatgcatctatgttggcatg

SEQ ID NO: 68 - DNA, 189 bp, Artificial Sequence
  Description of Artificial Sequence; note = synthetic construct
gcaacagatttggtgctggatgacagcaagaggctggccaagaggaagctgatagaggagaaccgggagaaaagacggcgggaagagctgcagaagtccatcgggcacaagccagagcccacagacgaggaatgggagctcatcaaaactgtcaccgaagcccatgtggcgaccaacgcccaaggcagc

SEQ ID NO: 69 - protein, 462 aa, Artificial Sequence
  Description of Artificial Sequence; note = synthetic construct
MDTKHFLPLDFSTQVNSSLTSPTGRGSMAAPSLHPSLGPGIGSPGQLHSPISTLSSPINGMGPPFSVISSPMGPHSMSVPTTPTLGFSTGSPQLSSPMNPVSSSEDIKPPLGLNGVLKVPAHPSGNMASFTKHICAICGDRSSGKHYGVYSCEGCKGFFKRTVRKDLTYTCRDNKDCLIDKRQRNRCQYCRYQKCLAMGMKREAVQEERQRGKDRNENEVESTSSANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 70 - DNA, 5449 bp, Artificial Sequence
  Description of Artificial Sequence; note = synthetic construct
gcgccgggggccgccgcgcccgccgcccgctgcctgcgccgccggccgggcatgagttagtcgcagacatggacaccaaacatttcctgccgctcgatttctccacccaggtgaactcctccctcacctccccgacggggcgaggctccatggctgccccctcgctgcacccgtccctggggcctggcatcggctccccgggacagctgcattctcccatcagcaccctgagctcccccatcaacggcatgggcccgcctttctcggtcatcagctcccccatgggcccccactccatgtcggtgcccaccacacccaccctgggcttcagcactggcagcccccagctcagctcacctatgaaccccgtcagcagcagcgaggacatcaagccccccctgggcctcaatggcgtcctcaaggtccccgcccacccctcaggaaacatggcttccttcaccaagcacatctgcgccatctgcggggaccgctcctcaggcaagcactatggagtgtacagctgcgaggggtgcaagggcttcttcaagcggacggtgcgcaaggacctgacctacacctgccgcgacaacaaggactgcctgattgacaagcggcagcggaaccggtgccagtactgccgctaccagaagtgcctggccatgggcatgaagcgggaagccgtgcaggaggagcggcagcgtggcaaggaccggaacgagaatgaggtggagtcgaccagcagcgccaacgaggacatgccggtggagaggatcctggaggctgagctggccgtggagcccaagaccgagacctacgtggaggcaaacatggggctgaaccccagctcgccgaacgaccctgtcaccaacatttgccaagcagccgacaaacagcttttcaccctggtggagtgggccaagcggatcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttaggcctgcgggcccatcctttgtgcccacccgttctggccaccctgcctggacgccagctgttcttctcagcctgagccctgtccctgcccttctctgcctggcctgtttggactttggggcacagcctgtcactgctctgcctaagagatgtgttgtcaccctccttatttctgttactacttgtctgtggcccagggcagtggctttcctgaggcagcagccttcgtggcaagaactagcgtgagcccagccaggcgcctccccaccgggctctcaggacaccctgccacaccccacggggcttgggcgactacagggtcttcgggccccagccctggagctgcaggagttgggaacggggcttttgtttccgttgctgtttatcgatgctggttttcagaattcctgtgtggccctcctgtctggagtgacatcttcatctgctctgaatactggtgcccagccagcccgtgacagcttccccctaatcaggaggggacagctgggggcgcaagctggtgtgtcatcagcaaagacctcagccgcctcggggatgagaggggactcgtggggcaagcaagctgccctgtgctctgagtgagggggaaggtagcccctttttccaaagataactcacagttttgccctcgagccaatgagaacatgagctgccctctgtgcaaggtttcggggccacctccaggctgcaggggcgggtcactcacccccctgttttctctctgccttggtgttctggtttcagactcccgactccccgttcagaccagagtgccccggcccctccccagcctgagtcttctccttgctctgcggggtgggctgaggcttgtccttgtttcctgcagggctggccctggctcgggcagggtggggcatcaccacctcactggccttgctggaggcacagggctctgcggacctgcagccatctgtgaggcccgcggggatgggaggggaggagggtggcctgttggtttccctcagagggggcaggtggcctggagagagaggggctcaggaactgggagcctcgtgggtggggcagatgctccgcggcctggagtggctctgccggggcattggtgggacccctgctcaggccttctctctggctgccagttgtgtctaaaagactcttggaatctgagaacccggagtcgcagcgccctcgggcctgggccacacgcaggccctggtgggaccacccagcctggtattgtccacggacagcgttgttcacccagagccttacttgggagcctcactgaacgcctgctctggttgaaggtggggtgggggcggggcttggggcctccctggctcagcccagtgcggcctggcgctcctcccgcaggctctgcccccgggctccggtggtgcggggccctctcaggttgaactcgcctcttttgcactggaaggccctccctttggcctgagtacttttcccgttcacgcctcagtcccgtggacccagcctttgtcagtggcaggtgcctgaacagagggtggatgggggggataccggagggggtcttgtcttcccagccgcagtctaggaatgatgcgggggggtggacgccttctccatagtctttccccacctggagcaggggcttcctcagtggtgaggggagctgcctacaggttggaccgggaggcagtggcttggagaggcagctttccagccttggtggggaagaaagtgtccattctttgccttcctggagctcccagccagagctgagcttaggcacccgagtggagcctgcagctgagtctgtgcccgagacaggctgtcagagattccagaagcctctcctccccgccgccctccacccctgcctttcagcgttgtggatccctagaggtggccccctgcccgatccaccgtcctgaggcagagtgttgagcctcatacctgtaccaggtccccggccagctgggcccctcccaggcactgccaggaagccccagctgcccctggcgggtgtggtggaaatggcaggagggtgcaggtactcttggggccccagcggtgggagtgcaaaagacccaacgccaacacctggtgccttttgcagccagcgcccacccatccgtgcccggacccttgggaatgcccgcggctccagaggaaaaagcccagggacggggcctccgttgcggggggtcggctgcttcttgggaactttgtcgtttccggcgctggctggctggctggctgtaaagcactgaagccccccggccgccaacccctgaaagcagaacctggcctccctggccacagcagccttacccaccgctctacgtgtcccgggcacttcccgcagccttcccgtccctttctcatcggccttgtagttgtacagtgctgttggtttgaaaaggtgatgtgtggggagtgcggctcatcactgagtagagaggtagaatttctatttaaccagacctgtagtagtattaccaatccagttcaattaaggtgattttttgtaattattattattttggtgggacaatctttaattttctaaagatagcactaacatcagctcattagccacctgtgcctgtccccgccttggcccggctggatgaagcggcttccccgcagggcccccacttcccagtggctgcttcctggggacccagggcaccccggcaccttcaggcacgctcctcagctggtcacctcccggctttgccgttcagatggggctcctgaggctcaggagtgaagatgccacagagccgggctcccctaggctgcgtcgggcatgcttggaagctggcctgccaggaccttccaccctggggcctgtgtcagccgccggccctccgcaccctggaagcacacggcctctgggaaggacagccctgaccttcggttttccgagcacggtgtttcccaagaattctgggctggcggcctggtggcagtgctggagatgaccccgagcccctccccgtggggcacccaggagggccctgccggaatgtgcagcctgtgggtagtcggctggtgtccctgtcgtggagctggggtgcgtgatctggtgctcgtccacgcaggtgtgtggtgtaaacatgtatgtgctgtacagagagacgcgtgtggagagagccgcacaccagcgccacccaggaaaggcggagcggttaccagtgttttgtgtttatttttaatcaagacgtttcccctgttttcctataaatttgcttcgtgtaagcaagtacataaggaccctcctttggtgaaatccgggttcgaatgaatatctcaaggcaggagatgcatctattttaagatgctttggagcagacagctttagccgttcccaatccttagcaatgccttagctgggacgcatagctaatactttagagaggatgacagatccataaagagagtaaagataagagaaaatgtctaaagcatctggaaaggtaaaaaaaaaaaatctatttttgtacaaatgtaattttatccctcatgtatacttggatatggcggggggagggctgggactgtttcgtttctgcttctagagattgaggtgaaagcttcgtccgagaaacgccaggacagacgatggcagaggagagggctcctgtgacggcggcgaggcttgggaggaaaccgccgcaatgggggtgtcttccctcggggcaggagggtgggcctgaggctttcaagggttttcttccctttcgagtaatttttaaagccttgctctgttgtgtcctgttgccggctctggccttcctgtgactgactgtgaagtggcttctccgtacgattgtctctgaaacatcgtggcctcaggtgccagggtttgatggacagtagcattagaattgtggaaaaggaacacgcaaagggagaagtgtgagaggagaaacaaaatatgagcgtttaaaatacatcgccattcagttcgttaaaaaaaaaaaaaaaaaaaaaaaaa

SEQ ID NO: 71 - DNA, 210 bp, Artificial Sequence
  Description of Artificial Sequence; note = synthetic construct
accaagcacatctgcgccatctgcggggaccgctcctcaggcaagcactatggagtgtacagctgcgaggggtgcaagggcttcttcaagcggacggtgcgcaaggacctgacctacacctgccgcgacaacaaggactgcctgattgacaagcggcagcggaaccggtgccagtactgccgctaccagaagtgcctggccatgggcatg

SEQ ID NO: 72 - DNA, 177 bp, Artificial Sequence
  Description of Artificial Sequence; note = synthetic construct
aagcgggaagccgtgcaggaggagcggcagcgtggcaaggaccggaacgagaatgaggtggagtcgaccagcagcgccaacgaggacatgccggtggagaggatcctggaggctgagctggccgtggagcccaagaccgagacctacgtggaggcaaacatggggctgaaccccagc

SEQ ID NO: 73 - protein, 462 aa, Artificial Sequence
  Description of Artificial Sequence; note = synthetic construct
MASNSSSCPTPGGGHLNGYPVPPYAFFFPPMLGGLSPPGALTTLQHQLPVSGYSTPSPATIETQSSSSEEIVPSPPSPPPLPRIYKPCFVCQDKSSGYHYGVSACEGCKGFFRRSIQKNMVYTCHRDKNCIINKVTRNRCQYCRLQKCFEVGMSKESVRNDRNKKKKEVPKPECSESYTLTPEVGELIEKVRKAHQETFPALCQLGKYTTNNSSEQRVSLDIDLWDKFSELSTKCIIKTVEFAKQLPGFTTLTIADQITLLKAACLDILILRICTRYTPEQDTMTFSDGLTLNRTQMHNAGFGPLTDLVFAFANQLLPLEMDDAETGLLSAICLICGDRQDLEQPDRVDMLQEPLLEALKVYVRKRRPSRPHMFPKMLMKITDLRSISAKGAERVITLKMEIPGSMPPLIQEMLENSEGLDTLSGQPGGGGRDGGGLAPPPGSCSPSLSPSSNRSSPATHSP

SEQ ID NO: 74 - DNA, 2907 bp, Artificial Sequence
  Description of Artificial Sequence; note = synthetic construct
gccatctgggcccaggccccatgccccgaggaggggtggtctgaagcccaccagagccccctgccagactgtctgcctcccttctgactgtggccgcttggcatggccagcaacagcagctcctgcccgacacctgggggcgggcacctcaatgggtacccggtgcctccctacgccttcttcttcccccctatgctgggtggactctccccgccaggcgctctgaccactctccagcaccagcttccagttagtggatatagcacaccatccccagccaccattgagacccagagcagcagttctgaagagatagtgcccagccctccctcgccaccccctctaccccgcatctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaaggagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtgaccgcccacgccacatggacacagccctcgccctccgccccggcttttctctgcctttctaccgaccatgtgaccccgcaccagccctgcccccacctgccctcccgggcagtactggggaccttccctgggggacggggagggaggaggcagcgactccttggacagaggcctgggccctcagtggactgcctgctcccacagcctgggctgacgtcagaggccgaggccaggaactgagtgaggcccctggtcctgggtctcaggatgggtcctgggggcctcgtgttcatcaagacacccctctgcccagctcaccacatcttcatcaccagcaaacgccaggacttggctcccccatcctcagaactcacaagccattgctccccagctggggaacctcaacctcccccctgcctcggttggtgacagagggggtgggacaggggcggggggttccccctgtacataccctgccataccaaccccaggtattaattctcgctggttttgtttttattttaatttttttgttttgatttttttaataagaattttcattttaagcacatttatactgaaggaatttgtgctgtgtattggggggagctggatccagagctggagggggtgggtccgggggagggagtggctcggaaggggcccccactctcctttcatgtccctgtgccccccagttctcctcctcagccttttcctcctcagttttctctttaaaactgtgaagtactaactttccaaggcctgccttcccctccctcccactggagaagccgccagcccctttctccctctgcctgaccactgggtgtggacggtgtggggcagccctgaaaggacaggctcctggccttggcacttgcctgcacccaccatgaggcatggagcagggcagagcaagggccccgggacagagttttcccagacctggctcctcggcagagctgcctcccgtcagggcccacatcatctaggctccccagcccccactgtgaaggggctggccaggggcccgagctgcccccacccccggcctcagccaccagcacccccatagggcccccagacaccacacacatgcgcgtgcgcacacacacaaacacacacacactggacagtagatgggccgacacacacttggcccgagttcctccatttccctggcctgccccccacccccaacctgtcccacccccgtgccccctccttaccccgcaggacgggcctacaggggggtctcccctcacccctgcacccccagctgggggagctggctctgccccgacctccttcaccaggggttggggccccttcccctggagcccgtgggtgcacctgttactgttgggctttccactgagatctactggataaagaataaagttctatttattct

SEQ ID NO: 75 - DNA, 216 bp, Artificial Sequence
  Description of Artificial Sequence; note = synthetic construct
atctacaagccttgctttgtctgtcaggacaagtcctcaggctaccactatggggtcagcgcctgtgagggctgcaagggcttcttccgccgcagcatccagaagaacatggtgtacacgtgtcaccgggacaagaactgcatcatcaacaaggtgacccggaaccgctgccagtactgccgactgcagaagtgctttgaagtgggcatgtccaag

SEQ ID NO: 76 - DNA, 207 bp, Artificial Sequence
  Description of Artificial Sequence; note = synthetic construct
gagtctgtgagaaacgaccgaaacaagaagaagaaggaggtgcccaagcccgagtgctctgagagctacacgctgacgccggaggtgggggagctcattgagaaggtgcgcaaagcgcaccaggaaaccttccctgccctctgccagctgggcaaatacactacgaacaacagctcagaacaacgtgtctctctggacattgacctc

SEQ ID NO: 77 - protein, 427 aa, Artificial Sequence
  Description of Artificial Sequence; note = synthetic construct
MEAMAASTSLPDPGDFDRNVPRICGVCGDRATGFHFNAMTCEGCKGFFRRSMKRKALFTCPFNGDCRITKDNRRHCQACRLKRCVDIGMMKEFILTDEEVQRKREMILKRKEEEALKDSLRPKLSEEQQRIIAILLDAHHKTYDPTYSDFCQFRPPVRVNDGGGSHPSRPNSRHTPSFSGDSSSSCSDHCITSSDMMDSSSFSNLDLSEEDSDDPSVTLELSQLSMLPHLADLVSYSIQKVIGFAKMIPGFRDLTSEDQIVLLKSSAIEVIMLRSNESFTMDDMSWTCGNQDYKYRVSDVTKAGHSLELIEPLIKFQVGLKKLNLHEEEHVLLMAICIVSPDRPGVQDAALIEAIQDRLSNTLQTYIRCRHPPPGSHLLYAKMIQKLADLRSLNEEHSKQYRCLSFQPECSMKLTPLVLEVFGNEIS

SEQ ID NO: 78 - DNA, 1284 bp, Artificial Sequence
  Description of Artificial Sequence; note = synthetic construct
atggaggcaatggcggccagcacttccctgcctgaccctggagactttgaccggaacgtgccccggatctgtggggtgtgtggagaccgagccactggctttcacttcaatgctatgacctgtgaaggctgcaaaggcttcttcaggcgaagcatgaagcggaaggcactattcacctgccccttcaacggggactgccgcatcaccaaggacaaccgacgccactgccaggcctgccggctcaaacgctgtgtggacatcggcatgatgaaggagttcattctgacagatgaggaagtgcagaggaagcgggagatgatcctgaagcggaaggaggaggaggccttgaaggacagtctgcggcccaagctgtctgaggagcagcagcgcatcattgccatactgctggacgcccaccataagacctacgaccccacctactccgacttctgccagttccggcctccagttcgtgtgaatgatggtggagggagccatccttccaggcccaactccagacacactcccagcttctctggggactcctcctcctcctgctcagatcactgtatcacctcttcagacatgatggactcgtccagcttctccaatctggatctgagtgaagaagattcagatgacccttctgtgaccctagagctgtcccagctctccatgctgccccacctggctgacctggtcagttacagcatccaaaaggtcattggctttgctaagatgataccaggattcagagacctcacctctgaggaccagatcgtactgctgaagtcaagtgccattgaggtcatcatgttgcgctccaatgagtccttcaccatggacgacatgtcctggacctgtggcaaccaagactacaagtaccgcgtcagtgacgtgaccaaagccggacacagcctggagctgattgagcccctcatcaagttccaggtgggactgaagaagctgaacttgcatgaggaggagcatgtcctgctcatggccatctgcatcgtctccccagatcgtcctggggtgcaggacgccgcgctgattgaggccatccaggaccgcctgtccaacacactgcagacgtacatccgctgccgccacccgcccccgggcagccacctgctctatgccaagatgatccagaagctagccgacctgcgcagcctcaatgaggagcactccaagcagtaccgctgcctctccttccagcctgagtgcagcatgaagctaacgccccttgtgctcgaagtgtttggcaatgagatctcctga

SEQ ID NO: 79 - DNA, 210 bp, Artificial Sequence
  Description of Artificial Sequence; note = synthetic construct
cggatctgtggggtgtgtggagaccgagccactggctttcacttcaatgctatgacctgtgaaggctgcaaaggcttcttcaggcgaagcatgaagcggaaggcactattcacctgccccttcaacggggactgccgcatcaccaaggacaaccgacgccactgccaggcctgccggctcaaacgctgtgtggacatcggcatgatgaag

SEQ ID NO: 80 - DNA, 195 bp, Artificial Sequence
  Description of Artificial Sequence; note = synthetic construct
gagttcattctgacagatgaggaagtgcagaggaagcgggagatgatcctgaagcggaaggaggaggaggccttgaaggacagtctgcggcccaagctgtctgaggagcagcagcgcatcattgccatactgctggacgcccaccataagacctacgaccccacctactccgacttctgccagttccggcctcca

SEQ ID NO: 81 - protein, 51 aa, Artificial Sequence
  Description of Artificial Sequence; note = synthetic construct
MKGGVRKDRRGGRMLKHKRQRDDGEGRGEVGSAGDMRAANLWPSPLMIKRS

SEQ ID NO: 82 - protein, 47 aa, Artificial Sequence
  Description of Artificial Sequence; note = synthetic construct
MARRPRHSIYSSDEDDEDFEMCDHDYDGLLPKSGKRHLGKTRWTREE

SEQ ID NO: 83 - DNA, 71 bp, Artificial Sequence
  Description of Artificial Sequence; note = synthetic construct
ctctggatccggtggaggtggttctggaggaggtggttccggaggtggaggaaaggagacgcgttacgctg

SEQ ID NO: 84 - protein, 24 aa, Artificial Sequence
  Description of Artificial Sequence; note = synthetic construct
LWIRWRWFWRRWFRRWRKGDALTL

(19) United States
(12) Patent Application Publication
(10) Pub. No.: US 2007/0192880 A1
(43) Pub. Date: Aug. 16, 2007

(54) Horming response element binding transregulators

(75) Inventors: Mesut Muyan, Rochester, NY (US); Jing Huang, Rochester, NY (US)

Correspondence Address: NEEDLE & ROSENBERG, P.C., SUITE 1000, 999 PEACHTREE STREET, ATLANTA, GA, 30309-3915, US

(73) Assignee: UNIVERSITY OF ROCHESTER, Rochester, NY (US)

(21) Appl. No.: 10/574,322

(22) PCT Filed: Oct. 4, 2004
(86) PCT No.: PCT/US04/32561

§ 371 Date: Oct. 24, 2006

Related U.S. Application Data
(60) Provisional application No. 60/508,463, filed on Oct. 3, 2003.

(51) Int. Cl. A01K 67/027; A61K 48/00; C07H 21/04; C12P 21/06; C07K 14/82

(52) U.S. Cl. 800/14; 536/23.5; 530/350; 435/69.1; 435/320.1; 435/325; 514/44

(57) ABSTRACT

Disclosed are compositions and methods for ERE-binding tansregulators that specifically and potently regulate ERE-containing genes. To accomplish this, we took advantage of the modular nature of ER and initially designed a monomeric ERE binding module by co-joining two DNA binding domains with the hinge domain. Integradon of strong activation or repressor domains from other transcription factors into this module generated constitutively active ERE-binding activators (EBAs) and ERE-binding repressors (EBRs) respectively. These novel transregulators are the basis for the targeted regulation of ERE containing genes, the identification of estrogen reponsive gene networks, and the development of alternative/complementary therapeutic approaches for estrogen target tissue cancers.

This application claims benefit of U.S. Provisional Application No. 60/508,76, filed Oct. 3, 2003, which is incorporated herein by reference in its entirety.

### I. BACKGROUND OF THE INVENTION

Transcriptional regulation is very important in controlling cell growth. Transcription can be regulated by molecules that act as activators of transcription or as repressors of transcription. Disclosed are engineered modulators of transcription that are based on the covalent linking of regions that bind DNA. These molecules can then have repressor domains or activation domains added to modulate transcription. These molecules can be used to identify genes that are naturally controlled by certain transcription regulators, such as the estrogen receptor. Furthermore, they can be used to control transcription in cells at specific sites.

### II. SUMMARY OF THE INVENTION

In accordance with the purposes of this invention, as embodied and broadly described herein, this invention, in one aspect, relates to hormone response element (HRE)-binding transregulators.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### III. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows the construction and biochemical properties of ERE binding proteins. FIG. 1A shows the cDNA for the C domain encoding residues 181-263 of ERα, and for the CD domain, residues 181-301, were joined for the corresponding cDNA of an ERE binding module, CC, CCD or CDC. The cDNA of an ERE binding module is flanked by multiple cloning sites (MCS) for the subsequent insertions of cDNAs for single or multiple activation domains. FIG. 1B shows that cDNAs were transcribed and translated in vitro (TNT) in the presence of [³⁵S]-Met. Equal aliquots of reaction mixtures were subjected to 4-18% gradient SDS-PAGE followed by fluorography. V denotes the parent vector. Molecular weight markers in KDa are shown. FIG. 1C shows the abilities of proteins synthesized in vitro in the presence of unlabeled Met to bind to the consensus ERE were assessed by EMSA. Equal aliquots of reaction mixtures were incubated with 0.05 nM [³²p] end-labeled DNA in the absence (−) or presence (+) of a Flag antibody and resolved by 8% native PAGE. Free denotes the unbound radiolabeled ERE, while P-ERE indicates protein-ERE complexes. The ERα-ERE complex migrates as a doublet in EMSA, which is likely due to a truncated, post-translationally modified or monomeric ER that is capable of interacting with the ERE. This is also the case for the recombinant ERα observed in panel H. FIG. 1D shows the TNT reaction containing ERα or CDC shows no binding to the radiolabeled DNA fragment bearing a half-site ERE, 5′-GGTCA-3′. FIG. 1E shows fluorographic detection of radiolabeled CDC, ERα, the DNA binding defective C*DC, or the DNA binding defective ERα*. FIG. 1F shows binding of equal amounts of TNT reaction containing CDC, C*DC, ERα or ERα* proteins to the ERE was assessed by EMSA. FIG. 1G shows the identity of critical contact sites in the consensus ERE by ERα and CDC was assessed by the missing nucleoside hydroxyl radical assay followed by denaturing PAGE analysis. Lanes 1 and 6 indicate ERE containing no protein, whereas lane 2 and 5 represent reactions containing CDC and ERα, respectively. Lanes 3 and 4 represent the Maxam-Gilbert reaction (G). The lane 7 (C) represents DNA subjected to hydroxyl radical treatment and the lane 8 indicates the uncut DNA (U) in the absence of protein. The ratio of free (F) to bound (B) DNA at each base was quantified and plotted. Ratios are represented as horizontal bars, the length of which approximates the strength of nucleoside contact with the protein. The ERE half-sites are boxed. A representative autoradiogram of several independent experiments is shown. FIG. 1H shows the displacement of the radiolabeled ERE bound to CDC or ERα by unlabeled ERE. The construct-bound radiolabeled consensus ERE (0.05 nM) was incubated with 0, 0.125, 0.25, 0.5, 1, 2, 4 and 8 nM unlabeled ERE. Bound and free fractions quantified by PhosphorImager were used in the estimation of dissociation constant (K_{d}). A representative image of three independent experiments is shown. FIG. 1I shows CDC competes with ERα to bind to ERE. ERα was incubated with the end-labeled consensus ERE for 30 min. The CDC at 1, 8 and 16-fold more molar concentrations was then added into the reaction. Reactions were further incubated for 30 min and resolved with 8% non-denaturing PAGE.

FIG. 2 shows intracellular localization and transcription activation abilities of ERα and ERE binding proteins in transiently transfected COS-1 cells. FIG. 2A shows ERE binders, as ERα, are localized in the nucleus of transiently transfected COS-1 cells. Proteins were probed with a Flag antibody followed by a fluorescein-conjugated secondary antibody for visualization (FITC). DAPI staining indicates the nucleus. FIG. 2B shows that in order to examine the transactivation abilities of constructs, cells were transfected with 300 ng expression vectorper well bearing none (V) or cDNA for ERα, CDC, VP-16, CDC-VP16, ERα*, C*DC-VP16, or CDC-VP16* cDNA together with 125 ng of reporter plasmid. (*) denotes mutants with an impaired DNA binding finction, while VP16* depicts the activation function defective construct. The reporter plasmid contained either one (1× ERE) or two copies (2× ERE) of the consensus ERE in tandem located upstream of a simple promoter, TATA box, driving the expression of the firefly luciferase cDNA. The transfection efficiency was monitored by the co-expression of 0.5 ng of a reporter plasmid, pCMV-RL that drives the expression of Renilla luciferase cDNA. Cells were treated with or without 10⁻⁹ M E2 for 24 h. The cell extracts were assayed for luciferase enzymes, and the normalized firefly/Renilla luciferase activities are presented as fold changes compare to the control, which was set to one. Shown are the mean±SEM of three independent experiments performed in duplicate.

FIG. 3 shows the effects of the promoter-type on the ability of an EBA to transactivate the ERE-driven reporter gene in COS-1 cells. Cells were transiently transfected with an expression vector bearing no cDNA (V), cDNA for ERα or for an EBA together with a single ERE-driven TATA box (ERE-TATA) or the thymidine kinase (ERE-TK) promoter for the expression of the firefly luciferase cDNA. Shown are the mean±SEM of three independent experiments performed in duplicate.

Table 1. The effect of increasing number of ADs and ERE on transactivation abilities of EBAs. COS-1 cells were transiently transfected with an expression vector bearing no cDNA (V) or cDNA for ERα, EBAs with single or two ADs of VP 16 or of p65 fused to amino-, carboxyl- or both termini. Cells were also co-transfected with the reporter vectors bearing one (1×ERE) or two copies (2×ERE) of the consensus ERE located upstream of a simple TATA box promoter that drives the expression of the firefly luciferase cDNA. The mean±SEM represents three independent experiments performed in duplicate.

Table 2. Effects of the cell-type on transactivation abilities of EBAs. CHO or MDA-MB-231 cells were transiently transfected with an expression vector bearing no cDNA (V), cDNA for ERα or for an EBA. The reporter plasmid contained either one (1×ERE) or two copies (2×ERE) of the consensus ERE located upstream of the TATA box promoter driving the expression of the firefly luciferase cDNA. The mean±SEM of three independent experiments performed in duplicate indicates normalized luciferase values represented as fold change.

FIG. 4 shows transcriptional responses from non-consensus ERE and non-ERE sequences in COS-1 cells. FIG. 4A shows cells were transfected with expression vectors bearing none (V), ERα, (VP16)₂-CDC-(p65)₂ or (p65)₂-CDC-(VP16)₂ cDNA and treated with 10⁻⁹ M E₂ for 24 h. pS2, Oxytocin or Lactoferrin depicts 5′-GGTCAcggTGGCC-3′, 5′-GGTGAcctTGACC-3′ or 5′GGTCAaggCGATC-3′ ERE sequence derived from the human pS2, oxytocin or lactoferrin gene ERE, respectively. An ERE drives the expression of the firefly luciferase cDNA as the reporter enzyme from the TATA box promoter. FIG. 4B shows COS-1 cells were transfected with expression vectors bearing ERα, or (p65)₂-CDC-(VP16)₂ cDNA together with the TATA box reporter vector. The reporter vector contains no (TATA) or two ERE half sites with 0, 1, 2, 3, 4, 5, 10 or 15 non-specific central nucleotides. FIGS. 4C and 4D show COS-1 cells were transfected with expression vectors bearing none (V), RXRα (Panel C) PR (Panel D), ERα, (VP16)₂-CDC-(p65)₂ or (p65)₂-CDC-(VP16)₂ cDNA. Cells were treated with 10⁻⁶ M 9-cis-retinoic acid for RXRα, 10⁻⁸ M Progesterone for PR or 10⁻⁹ M E₂ for ERα for 24 h. The TATA box reporter vector bears one consensus RXRE 5′-AGGTCAnAGGTCA-′3 (Panel C) or PRE 5′-AGAACAnnnTGTTCT-3′ (panel D) that drives the expression of the reporter firefly luciferase cDNA. Normalized luciferase values represented as fold change are the mean±SEM of three independent experiments performed in duplicate.

FIG. 5 shows the effects of EBAs on transcriptional responses from ERE-dependent and -independent reporter constructs, and on cell cycle in MDA-MB-231 cells. (A&B) Cells were transfected with expression vectors bearing none (V), ERα, CDC, p65-CDC-VP16 or (p65)₂-CDC-(VP16)₂ cDNA together with the pS2, C3 or Oxytocin (Oxy) promoter (A), or together with the Col or RARα promoter (B). All promoter constructs drive the expression of the firefly luciferase cDNA as the reporter enzyme. Cells were then treated without or with 10⁻⁹ M E₂ (shown only for ERα) for 24 h. Normalized luciferase values represented as fold change are the mean±SEM of three independent experiments performed in duplicate. FIG. 5C shows cells were transfected with pEGFP vector bearing none (EGFP), CDC, ERα or an EBA cDNA. One day after transfection, cells were subjected to a fluorescent-activated cell sorting (FACS) to separate EGFP-positive cell population. Cell cycle analysis was simultaneously performed with the EGFP-positive cells. The percentage of cells at G1 phase was assessed among treatment groups and expressed as percent change compared to cells transfected with EGFP vector bearing no cDNA. Shown is a representative experiment from several independent experiments.

FIG. 6 shows the effects of EBAs on transcriptional responses (A&B) from ERE-dependent and -independent reporter constructs, and on cell cycle (C) in MCF-7 cells. Transfection and processing of cells were done as described in legend of FIG. 5.

FIG. 7 shows EBRs effectively repress the transcription of reporter gene from an ERE-containing heterologous promoter in COS-1 and CHO cells. To examine the transrepression abilities of constructs, cells were transfected with 75 ng expression vector per well bearing none (V) or cDNA for ERα, CDC, an EBR together with 125 ng of reporter plasmid. The reporter SV40 enhancer/promoter vector containing one consensus ERE juxtaposed to the promoter that drives the firefly luciferase cDNA expression. The transfection efficiency was monitored by the co-expression of 0.5 ng of a reporter plasmid, pCMV-RL that drives the expression of Renilla luciferase cDNA. Cells were treated with 10⁻⁹ M E2, 10⁻⁷ M 4-OHT or 10⁻⁹ M E2+10⁻⁷ M 4-OHT for ERα, and without ligand for EBRs for 24 h. The cell extracts were assayed for luciferase enzymes, and the normalized firefly/Renilla luciferase activities are presented as fold changes compare to the control, which was set to 100. Shown are the mean±SEM of three independent experiments performed in duplicate.

FIG. 8 shows the effects of EBA to trans-repress the Catepsin D gene promoter in COS-1 cells. Transfection and processing of cells were done as described in legend of FIG. 7. The reporter vector contains the human Catepsin D (CatD) gene promoter that drives the firefly luciferase cDNA expression. The mean±SEM indicates three independent experiments performed in duplicate.

FIG. 9 shows that EBAs trans-repress the expression of reporter gene from a reporter vector bearing the human C3 gene promoter that drives the firefly luciferase cDNA expression in COS-1 cells. Transfection and processing of cells were done as described in legend of FIG. 7. Shown are the mean±SEM of three independent experiments performed in duplicate and expressed as fold changes compare to the parent expression vector (V), which was set to one.

FIG. 10 shows in the upper panels that the infection of MCF-7 and MDA-MB-231 cells with the recombinant adenovirus expressing the β-galactosidase cDNA at different MOI. Cells were fixed, subjected to an in situ β-galactosidase assay for blue color development. Lower panels indicate the induction of β-galactosidase activity as a function of time in cells infected with the recombinant adenovirus expressing β-galactosidase cDNA at MOI of 500 for MCF-7 and 250 for MDA-MB-231 cells. Images were captured by CCD camera attached to a phase-contrast microscope.

FIG. 11 shows the regulation of endogenous E2-responsive genes in MDA-MB-231 cells infected with recombinant adenovirus bearing no (CMV), ERα (E2-ERα) or p65-CDC-VP16 (PV) at MOI of 250 for 24 h. ERα infected cells were treated with 10⁻⁹ M E2 for 24 h. The ΔRn indicates the intensity of fluorescence signal as a function of PCR cycle number. An experiment performed in duplicate is shown.

FIG. 12 shows the cell cycle progression in MDA-MB-231 cells. Cells were infected with the recombinant adenovirus expressing no (CMV) or the ERα in the presence of a physiological concentration, 10⁻⁹ M, of E2, or PV cDNA. The data show that the transactivator PV, just as the E2-ERα, represses cell cycle progression in MDA-MB-231 cells by blocking G1/S phase transition.

### IV. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from ““about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

B. Compositions and Methods

The central dogma of molecular biology posits that genetic information flows from genes stored in DNA, to RNA, such as mRNA in eukaryotes, and then to proteins. While certain virsus, such as retroviruses, have a different cycle because their genetic material is stored in RNA, one of the most fundamental processes of life is the control of the movement of the information contained in genes to the expressed protein product encoded by those genes. There are many points of control along this pathway, including the point at which the DNA is transcribed to RNA, producing the molecule which will be used to produce the protein product. Control of this process is very important, as many diseases, most notably cancer, arise because of aberrant control of this process, wherein either the wrong gene is being expressed in the wrong cell or too much of a particular gene is being expressed, or not enough of a particular gene, such as a negative cell cycle regulator, is being expressed.

The process of turning the information in the DNA into RNA, is called transcription. The transcription process is a very complex and involves many different proteins, including the basal transcription machinery. The basal transcription machinery comprises more than 30 proteins and polypeptides. The basal transcription machinery functions at the start site, the TATA box, and is responsible for actually decoding the DNA and synthesizing the corresponding RNA of a given gene.

In addition to the basal transcription machinery, typically other proteins are required for adequate and controlled transcription. These other proteins are broadly referred to as transcription modulators or transcription factors, such as transcription activators or enhancers, or transcription repressors. Transcription modulators typically function by binding a region of the DNA associated with the gene called a DNA binding site. Typically these regions are in the 5′1 leader sequence of a gene. There are a number of different types of DNA binding sites, but there are characteristics that many DNA binding sites share. For example, DNA binding sites typically are made up of a precise DNA sequence, i.e. 5′-GGTCA-3′. It is possible that DNA binding sites can be made up of a set of sequences, i.e. 5′-GGTCA-3′ or 5′-GGCCA-3′, that are related, but have some differences. These differences can arise because, the contacts needed for binding between the protein and the DNA, either do not involve the variant position or because the variant position provides the appropriate contact with more than one specific nucleotide.

Many transcription modulators bind the DNA as dimers, meaning there are two modulators, which are typically interacting with each other, as well as interacting with the DNA. This generally allows increased affinity and specificity to take place between the modulator and the DNA. The affinity is generally determined by how many binding contacts are made between the modulator and the DNA. When there are two modulators, the number of binding contacts increases, not only between the modulators and the DNA, relative to a single modulator, but also binding contacts between the two modulators increase. Specificity arises from the type of contact, i.e. the orientation of the contacts between the modulator and the DNA. When modulators bind as dimers, they can do so as homodimers or heterodimers. A homodimer is a dimer that is formed between two identical transcription modulators, and a heterodimer is a dimer that is formed between two different transcription modulators.

As indicated, transcription modulators bind DNA, and the site that they bind DNA at is called a DNA binding site. As indicated, transcription modulators typically bind DNA as a dimer, in the natural context (i.e. the modulator may bind as a monomer, but in the natural state of the cell, the amount of the dimer is insufficient to bind the site as a monomer because of the low affinity, but does bind as a dimer, because of the increased affinity). As indicated, a dimer comprises two transcription modulators, and each of these modulators binds DNA at a DNA binding site in the DNA, which has a specific sequence or set of sequences. Thus, when a dimer binds, there will be two DNA binding sites, one for each monomer of the dimer. The combination of two sites can be referred to as an element, such as an enhancer element.

Typically transcription modulators are composed of different functional domains. Most transcription modulators have a DNA binding domain, as discussed above. In addition, transcription modulators also typically have a domain that imparts an effect on the basal transcription machinery, i.e. enhancement or repression. These domains can be called activation domains or repressor domains. In addition, there can be domains, for example, that cause nuclear localization, interact with other proteins, such as other modulators, or which interact with regulators of the modulator themselves. There can also be domains that connect the various domains of the modulator. Another type of domain is a dimerization domain. A dimerization domain, is a region of a transcription modulator that facilitates the interaction between two modulators, to form a dimer. Thus, the dimerization of two transcription modulators occurs through non-covalent interactions.

There is a need for engineered transcriptional modulators. This need exists because it can allow the designer control of transcription for genes of choice. Furthermore, there is a need to be able to identify sets of genes that are activated by the same modulators. Disclosed herein are engineered transcriptional modulators. In certain embodiments these engineered transcriptional modulators are called linked modulators. Linked modulators are composed of at least two DNA binding domains that are covalently linked. Linked modulators can also have at least one other domain which modulates transcription, called a modulation domain. In certain embodiments these linked modulators can have the effect of creating constitutive transcriptional activation from a defined element that typically would not be constitutively active because the typical transcriptional dimer modulator for the element site is not a constitutive activator, but for example, requires that the modulator interact with another molecule or protein before activation takes place. Disclosed are properties of the linked modulators and uses of the linked modulators.

1. Breast Cancer Treatments

a) Endocrine Therapy

Although complementation and convergence of various signaling pathways are ultimately responsible for the physiology and pathophysiology of breast tissue, it is clear that estrogens are primary agents in the development of most breast cancers by stimulating and maintaining malignant cell proliferation. Consequently, measures that perturb the estrogen environment of the tumor cells by blocking the synthesis of estrogen or by preventing estrogen actions are current strategies for therapeutic intervention for the neoplasm. The management of early breast cancer is primarily based on surgical removal of the tumor by mastectomy or lumpectomy without or with radiotherapy, followed by an adjuvant systemic therapy dependent upon the ER status.

(1) GnRH Antagonist

GnRH regulates the synthesis and secretion of LH and FSH from the anterior pituitary (Shalev, E. et al. (2003) J Obstet Gynaecol Can 25, 98-113). GnRH-stimulated gonadotropin secretion can be blocked with antagonists as well as agonists whose sustained delivery induces pituitary desensitization (Limonta, P. et al. (2001) Expert Opin Investig Drugs 10, 709-720). These compounds ultimately reduce the circulating levels of gonadotropins and subsequently gonadal steroid hormone synthesis and secretion. Termed medical castration, this effect is exploited in the treatments of sex hormone-dependent neoplasms that also include breast (Robertson, J. F. et al. (2003) Eur. J. Cancer 39, 861-869; Grundker, C. et al. (2003) Reprod Biol Endocrinol 1, 65). The GnRH agonist, goserelin, remains the treatment of choice for pre-menopausal patients with ER-positive breast cancers. It appears that a combination of goserelin and antiestrogenic compounds to produce an estrogen blockade is a more effective treatment regimen in prolonging progression-free survival than the use of a GnRH agonist alone (Robertson, J. F. et al. (2003) Eur. J. Cancer 39, 861-869; Grundker, C. et al. (2003) Reprod Biol Endocrinol 1, 65).

(2) Aromatase Inhibitors

Since, as described above, estrogens are synthesized from androgenic steroid substrates by the aromatase enzyme, an effective perturbation of enzyme activity provides the most specific effects on estrogen production. Two major classes of aromatase inhibitors have been developed and are currently in clinical use. Type 1 inhibitors are steroidal analogues of androstenedione and bind to the same site as androstenedione on the aromatase molecule. However, unlike androstenedione these analogues bind to the enzyme irreversibly and covalently, because of their conversion to reactive intermediates by aromatase (Simpson, E. R. et al. (2002) Recent Prog. Horm. Res. 57, 317-338; Santen, R. J. (2002) J. Clin. Endocrinol. Metab. 87, 3007-3012). Therefore, Type 1 inhibitors are now commonly known as enzyme inactivators that include formestane and exemestane. Since the recovery of enzyme activity depends on both the re-synthesis of enzyme and the pharmacokinetics of the drug, these types of inhibitors have the potential for selectivity for the enzyme target and long-term effectiveness. However, such steroidal structures also have the potential for hormonal activity (Simpson, E. R. et al. (2002) Recent Prog. Horm. Res. 57, 317-338; Santen, R. J. (2002) J. Clin. Endocrinol. Metab. 87, 3007-3012).

Type II inhibitors are non-steroidal compounds that are triazoles and include anastrozole and letrozole. These type II inhibitors bind reversibly to the enzyme and fit into the substrate-binding site such that azole nitrogens interact with the heme prosthetic group in the aromatase enzyme with high affinity and specificity (Simpson, E. R. et al. (2002) Recent Prog. Horm. Res. 57, 317-338; Santen, R. J. (2002) J. Clin. Endocrinol. Metab. 87, 3007-3012).

Aromatase inhibitors are not effective in pre-menopausal women, as lower circulating levels of estrogen could result in the stimulation of the hypothalamo-hypophyseal axis activity, which in turn increases circulating estrogen levels by enhancing estrogen synthesis from the ovaries (Simpson, E. R. et al. (2002) Recent Prog. Horm. Res. 57, 317-338; Santen, R. J. (2002) J. Clin. Endocrinol. Metab. 87, 3007-3012). Thus, application of aromatase inhibitors to treatment of pre-menopausal women with breast cancer is limited to their combined usage with goserelin. Since, however, the primary source of estrogen in post-menopausal women is the conversion of adrenal C19 steroids into estrogens by intra-tumor as well as extra-gonadal sites of aromatase activity, aromatase inhibitors constitute an effective therapeutic intervention for breast cancers (Simpson, E. R. et al. (2002) Recent Prog. Horm. Res. 57, 317-338; Santen, R. J. (2002) J. Clin. Endocrinol. Metab. 87, 3007-3012). Studies indicate that aromatase inhibitor therapy leads to a precipitous drop in the intratumoral concentrations of estrogens together with a corresponding loss of intratumoral aromatase activity (Simpson, E. R. et al. (2002) Recent Prog. Horm. Res. 57, 317-338; Santen, R. J. (2002) J. Clin. Endocrinol. Metab. 87, 3007-3012). Clinical trials have provided further support for the use of the aromatase inhibitors as first line treatment of ER positive breast cancers in post-menopausal women (Simpson, E. R. et al. (2002) Recent Prog. Horm. Res. 57, 317-338; Santen, R. J. (2002) J. Clin. Endocrinol. Metab. 87, 3007-3012). Since, however, aromatase inhibitors inhibit aromatase activity globally, these compounds could affect many other tissues wherein estrogens are required for normal function. The development of tissue-specific aromatase inhibitors could expand the utility of this approach in the treatment of breast cancers (Simpson, E. R. et al. (2002) Recent Prog. Horm. Res. 57, 317-338; Santen, R. J. (2002) J. Clin. Endocrinol. Metab. 87, 3007-3012).

(3) Antiestrogens

In addition to estrogen, ER also binds compounds that act as estrogen competitors (McDonnell, D. P. (1999) Trends Endocrinol Metab 10, 301-311; Jordan, V. C. et al. (1999) Endocr. Rev. 20, 253-278; Jensen, E. V. et al. (2003) Clin Cancer Res 9, 1980-1989). These compounds can be divided into two categories: Type I and II. Type I compounds include tamoxifen, toremifene and raloxifene and are now referred to as selective estrogen receptor modulators, SERMs. Tamoxifen and toremifene have a triphenylethylene structure and raloxifene has a benzothiophene structure. Although the primary structure of these SERMs differs significantly from that of estrogen which is a cyclophenanthrene, they have conformations that allow them to bind to ERs. SERMs can function as agonists or antagonists depending on ER subtypes, and the cells and tissues in which they operate (McDonnell, D. P. (1999) Trends Endocrinol Metab 10, 301-311; Wakeling, A. E. (2000) Endocr Relat Cancer 7, 17-28). Tamoxifen and raloxifene function as antagonists in breast. While tamoxifen acts as an agonist in the uterus, bone and cardiovascular system, raloxifen functions as a pure antagonist in the uterus but an agonist in bone.

Type II compounds that include steroidal compounds ICI 164,384 and ICI 182,780 are derivatives of estrogen with long alkyl 7α-substitutions and are considered as pure antagonists devoid of estrogenic activity in most experimental systems tested (McDonnell, D. P. (1999) Trends Endocrinol Metab 10, 301-311; Wakeling, A. E. et al. (2001) Clin Cancer Res 7, 4350s-4355s; discussion 4411s-4412s). The distinct pharmacological properties of these antiestrogens allow treatment regimens to be targeted to a specific tissue of interest to minimize unintended development of other tissue malignancies.

Biochemical, functional and structural studies have indicated that antiestrogens alter the conformation of the carboxyl-terminal regions of ERs (McDonnell, D. P. (1999) Trends Endocrinol Metab 10, 301-311; Wakeling, A. E. et al. (2001) Clin Cancer Res 7, 4350s-4355s; discussion 4411s-4412s). Ligand binding is accompanied by a major reorganization in the tertiary structure of the LBD. Key differences in receptor conformation in the presence of different ligands are an indication for a structural basis for antagonism. Agonist binding induces a conformational change in which the carboxyl terminal helix 12 (H12), containing the core region of AF2, is aligned over the ligand-binding cavity that is composed of helices 3, 5/6, and 11. This alignment results in the formation of a specific binding site for the consensus LXXLL motif of co-activators. Binding of the Type 1 antagonists to ER sterically interferes with H12 positioning in that H12 interacts with a hydrophobic groove composed of residues from helices 3 and 5. This distinct orientation of H12 partially buries residues in the groove necessary for AF-2 activity, thereby preventing co-factor recruitment (Brzozowski, A. M. et al. (1997) Nature 389, 753-758; Pike, A. C. et al. (1999) EMBO J. 18, 4608-4618).

In ICI-bound ER, the side chain of ICI completely prevents H12 from associating with the LBD. This disordered conformation is thought to lead to full antagonism that results in the destabilized ER structure leading to disruption of nuclear-cytoplasmic shuttling and increased receptor turnover (Dauvois, S. et al. (1992) Proc Natl Acad Sci USA 89, 4037-4041; Dauvois, S. et al. (1993) J. Cell Sci. 106 (Pt 4), 1377-1388). ICI 182,780 (Faslodex) is approved as a “second-line” hormonal therapy for post-menopausal women with ER-positive metastatic breast cancer (Howell, A. et al. (2000) Cancer 89, 817-825).

Although blocking the AF-2 function by antagonists suggests a passive role for the prevention of ER-mediated transactivation by antiestrogens, an active repression of gene transcription appears to be involved. Tamoxifen-ER is shown to recruit the co-repressors NCoR, SMRT (Lavinsky, R. M. et al. (1998) Proc. Natl. Acad. Sci. USA 95, 2920-2925; Shang, Y. et al. (2000) Cell 103, 843-852) and REA (Delage-Mourroux, R. et al. (2000) J. Biol. Chem. 275, 35848-35856) to the promoters of estrogen responsive genes. The subsequent recruitment of histone deacetylases (HDACs) to the repressor-ER complex causes deacetylation of histone proteins. This event leads to chromatin compaction and transcriptional repression.

How does a SERM display partial agonist activity in an ER subtype and cell context dependent manner? The partial agonist activity of an antagonist is manifested as transcriptional responses from ERE-dependent genomic signaling pathway that are siginficantly lower than those observed with the estrogen-ER complex. The partial agonistic effect of SERMs, particularly tamoxifen, bound ERα, but not ERβ, from the ERE-dependent signaling pathway is modulated through the amino terminal AF-1 (Berry, M. et al. (1990) EMBO J. 9, 2811-2818; Yi, P. et al. (2002) Mol. Endocrinol. 16, 1810-1827). It appears that although the binding of a SERM to ERα prevents the AF-2 domain of the receptor from interacting with co-factors, the ability of the AF-1 domain to recruit the p160 family of co-factors in a cell-context dependent manner provides a mechanism for the partial agonistic effect of an antagonist for ERα (Yi, P. et al. (2002) Mol. Endocrinol. 16, 1810-1827; Webb, P. et al. (1998) Mol. Endocrinol. 12, 1605-1618; Yi, P. et al. (2002) Mol. Endocrinol. 16, 674-693). Studies have shown that the tamoxifen-bound ERα recruits co-repressors, but not co-activators, to target promoters in breast cancer cells (Lavinsky, R. M. et al. (1998) Proc. Natl. Acad. Sci. USA 95, 2920-2925; Shang, Y. et al. (2000) Cell 103, 843-852; Shang, Y. et al. (2002) Science 295, 2465-2468; Lee, E. J. et al. (2001) Mol. Med. 7, 773-782). On the other hand, the tamoxifen-ERα complex interacts preferentially with the p160 family co-activators as well as co-repressors to target promoters to stimulate transcription in cells derived from endometrium (Shang, Y. et al. (2002) Science 295, 2465-2468). This allows the tamoxifen-ERα complex to induce transcription, albeit at lower levels than estrogen-ERα, from estrogen responsive genes. Since the relative and absolute levels of expression of co-regulators vary among estrogen target cells, a balance between cell specific co-activators and co-repressors recruited by the antagonist-ERα complex appears to underlie the tissue selective pharmacology of SERMs (Shang, Y. et al. (2002) Science 295, 2465-2468; McKenna, N. J. et al. (1999) Endocr. Rev. 20, 321-344).

It should be noted that antiestrogens could also affect the function of intracellular proteins and signaling independently from ER signaling pathways. These include changes in oxidative stress responses, activation of specific protein kinase C isoforms as well as alterations in calmodulin finction and in cell membrane structure/function (Clarke, R. et al. (2001) Pharmacol. Rev. 53, 25-71).

b) Endocrine Resistance

Current therapeutic approaches for breast cancer treatment utilize endocrine measures to counteract the effects of estrogens and are often successful in the remission of tumors (Nicholson, R. I. et al. (2000) Br. J. Cancer 82, 501-513; Clarke, R. et al. (2001) J. Steroid Biochem. Mol. Biol. 76, 71-84; Nicholson, R. I. et al. (2003) Breast Cancer Res. Treat. 80 Suppl 1, S29-34; discussion S35; Clarke, R. et al. (2003) Oncogene 22, 7316-7339). However, one-third of breast cancers fails to respond to endocrine therapy (de novo endocrine resistance). Moreover, the beneficial effects of antiestrogens are counteracted by the capacity of tumor cells to eventually circumvent such therapies, allowing the tumor cells to resume growth (acquired endocrine resistance).

(1) De Novo Endocrine Resistance

The most important factor in de novo resistance to endocrine therapies is the lack of ER expression. However, the ontology of de novo endocrine resistance cells is unclear. These populations could stem from ERα-negative epithelial cells that acquire autonomous growth properties. It is also possible that mitogenic changes in non-proliferate and ERα positive epithelial cells give rise to a phenotype that gains autonomous growth but loses its ability to express the ERα gene. Although the status of the ERβ gene expression remains unknown in de novo resistant phenotypes, genetic alterations such as homozygous deletion, loss of heterozygosity or ERα gene mutation have not been reported to play a major role in the absence or loss of ER expression. Epigenetic control of ERα gene expression, on the other hand, appears to be critical for the absence/loss of the ERα gene transcription. CpG dinucleotides are frequently clustered into CpG islands and are often found in the promoters of genes (Chen, D. et al. (1999) Science 284, 2174-2177; Yang, X. et al. (2001) Endocr Relat Cancer 8, 115-127). Methylation of cytosines in these islands is associated with the repression of gene transcription (Chen, D. et al. (1999) Science 284, 2174-2177; Yang, X. et al. (2001) Endocr Relat Cancer 8, 115-127). Studies have indicated that the ERα gene contains CpG islands in its promoter and first exon (Falette, N. S. et al. (1990) Cancer Res. 50, 3974-3978; Ottaviano, Y. L. et al. (1994) Cancer Res. 54, 2552-2555). These ERα CpG islands are unmethylated in normal breast tissue and ERα-positive tumor lines but they are methylated in about half of primary breast cancers and most ER-negative breast cancer cell lines (Ottaviano, Y. L. et al. (1994) Cancer Res. 54, 2552-2555; Piva, R. et al. (1989) Biochemistry International 19, 267-275). The methylation status of CpG islands is associated with reduced or absent ERα expression, consequently cessation of ER protein synthesis (Ottaviano, Y. L. et al. (1994) Cancer Res. 54, 2552-2555; Piva, R. et al. (1989) Biochemistry International 19, 267-275). DNA methylation is regulated by the members of DNA-cytosine methyltransferase (DNMT) family (Chen, D. et al. (1999) Science 284, 2174-2177; Yang, X. et al. (2001) Endocr Relat Cancer 8, 115-127). Studies have shown that methyltransferase inhibitors cause partial de-methylation and restoration of ERα mRNA expression and synthesis of functional ERα protein (Ferguson, A. T. et al. (1995) Cancer Res. 55, 2279-2283). A disregulated expression of DNMT in ERα-negative breast cancer cell lines is proposed to be associated with the ER-gene repression (Yang, X. et al. (2001) Endocr Relat Cancer 8, 115-127).

Methylation of the ERα gene is required but may not be sufficient for ERα gene repression. It appears that the acetylation status of the ERα gene also contributes to ERα gene silencing (Yang, X. et al. (2000) Cancer Res. 60, 6890-6894). Studies showed that an increase in the acetylation of histones and de-methylation of the ER CpG islands synergistically activate ERα expression (Yang, X. et al. (2001) Cancer Res. 61, 7025-7029). This suggests that DNMT and HDAC are key regulators of methylation-mediated ERα gene silencing. These findings also imply that DNMT and HDAC inhibitors could be potentially important in establishing hormone responsiveness, and consequently in breast cancer treatment.

The underlying mechanisms for the methylation and acetylation status of the ERα gene promoter are unclear. Studies showed that the activation of the growth factor signaling pathways in breast cancer cells results in down-regulation of ERα gene expression (Pietras, R. J. et al. (1995) Oncogene 10, 2435-2446; Kumar, R. et al. (1996) J. Cell. Biochem. 62, 102-112; Tang, C. K. et al. (1996) Cancer Res. 56, 3350-3358) through, at least in part, an enhanced deacetylase activity (Mazumdar, A. et al. (2001) Nat Cell Biol 3, 30-37). It is therefore possible that aberrant growth factor signaling is involved in the absence or loss of ER gene expression. Additionally, altered expression of transacting factors responsible for ERα transcription and/or abnormalities in post-transcriptional and translational processing of ERα could also contribute to the absence of ER synthesis (Weigel, R. J. et al. (1993) Cancer Res. 53, 3472-3474; Ferguson, A. T. et al. (1997) Crit. Rev. Oncog. 8, 29-46; Ferguson, A. T. et al. (1998) Cancer Treat. Res. 94, 255-278).

Whatever the underlying mechanisms for the absence or loss of the ERα gene expression might be, an autonomous regulation of cell growth defines de novo resistance malignancies. Several growth factors and their receptors that include EGF, FGF, IGF, and TGF families have been shown to be over-expressed and to act as autocrine growth stimulators for breast cancer cells (Nicholson, R. I. et al. (2000) Br. J. Cancer 82, 501-513; Clarke, R. et al. (2001) J. Steroid Biochem. Mol. Biol. 76, 71-84; Clarke, R. et al. (2003) Oncogene 22, 7316-7339). Increased expression of growth factor receptors correlates with the severity of the disease (Nicholson, R. I. et al. (2000) Br. J. Cancer 82, 501-513; Clarke, R. et al. (2001) J. Steroid Biochem. Mol. Biol. 76, 71-84; Clarke, R. et al. (2003) Oncogene 22, 7316-7339). Receptors for growth factors are trans-membrane tyrosine kinases that are linked to activation of MAPK and/or AKT signaling pathways critical for cellular transformation, cancer progression and resistance to endocrine therapy (Nicholson, R. I. et al. (2000) Br. J. Cancer 82, 501-513; Clarke, R. et al. (2001) J. Steroid Biochem. Mol. Biol. 76, 71-84; Clarke, R. et al. (2003) Oncogene 22, 7316-7339). Disrupting signal transduction by specifically modulating the activity of these trans-membrane tyrosine kinases, therefore, constitutes an important strategy in the development anticancer agents. This includes antibody therapy to block ligand binding to the receptors and administration of small molecule tyrosine kinase inhibitors to inhibit receptor tyrosine kinase activity.

The EGFR belongs to a family of tyrosine kinases that contains human epidermal growth factor receptor-1 (or HER1), HER2, HER3, and HER4 (Yarden, Y. (2001) Oncology 61 Suppl 2, 1-13). Receptor activation is mediated by homo- and hetero-dimerization among all four HER family members upon binding to various ligands. Dimerization results in receptor tyrosine phosphorylation that allows the binding of downstream signaling molecules leading to the activation of kinases. Heterodimerization of HERs provides further diversification and specificity of signal transduction. Moreover, many other growth factor receptors can phosphorylate and activate HERs. HERs also act as a conduit for multiple other signaling pathways through trans-phosphorylation. HER2 is over-expressed in approximately 30% of breast cancers with adverse clinical prognosis (Slamon, D. J. et al. (1989) Science 244, 707-712). Trastuzumab is a novel humanized monoclonal antibody that binds to the extracellular domain of HER2 (Modi, S. et al. (2002) Curr Oncol Rep 4, 47-55). This leads to receptor down-regulation, degradation and consequently to inhibition of cell growth. Trastuzumab is currently being used in clinical settings for the treatment of patients with HER2-positive metastatic breast cancer with significant benefits as monotherapy or in combination with chemotherapy (Vogel, C. L. et al. (2002) J. Clin. Oncol. 20, 719-726; Slamon, D. J. et al. (2001) N. Engl. J. Med. 344, 783-792). Similarly, a humanized monoclonal antibody BX-EGF that targets the extracellular domain of HER1 has entered clinical trials for breast cancer treatments (Modi, S. et al. (2002) Curr Oncol Rep 4, 47-55).

Small molecule compounds compete for the ATP-binding sites of the tyrosine kinase domains of the HER-family. Binding of these compounds to the receptor block the activation of the tyrosine kinase domain and subsequently prevent the downstream signaling cascades that include MAPK and AKT pathways (Modi, S. et al. (2002) Curr Oncol Rep 4, 47-55; Arteaga, C. L. et al. (2002) Semin. Oncol. 29, 4-10; Goel, S. et al. (2002) Curr Oncol Rep 4, 9-19). The two most clinically advanced compounds in this class of agents are ZD1839 and OSI-774 that specifically target HER1, whereas CI-1033 interacts with all four members of the HER-family. In pre-clinical models ZD1839 displays anti-proliferative activity by interfering with cell cycle progression in a wide range of HER-expressing cancer cell lines (Sliwkowski, M. X. et al. (1999) Semin. Oncol. 26, 60-70). ZD1839 also augments the antitumor effects of chemo- and radiation-therapies (Modi, S. et al. (2002) Curr Oncol Rep 4, 47-55; Arteaga, C. L. et al. (2002) Semin. Oncol. 29, 4-10; Goel, S. et al. (2002) Curr Oncol Rep 4, 9-19). However, recent clinical trials in patients with refractory metastatic breast cancer, suggest that EGFR inhibitor ZD1839 has no clinical activity (Arteaga, C. L. et al. (2004) Semin. Oncol. 31, 3-8). Pharmacodynamic studies (Arteaga, C. L. et al. (2004) Semin. Oncol. 31, 3-8) also indicate that the activated EGFR in breast tumor cells is indeed blocked by EGFR tyrosine kinase inhibitors but without an associated reduction in tumor cell proliferation. These results imply that 1) levels of P-EGFR do not predict for EGFR dependence nor sensitivity to therapeutic EGFR blockade, and 2) drug-induced inhibition of P-EGFR is not predictive of response to treatment either.

(2) Acquired Endocrine Resistance

Counteraction of the beneficial effects of endocrine approaches by the tumor cells that express ER leads to acquired endocrine resistance phenotypes, in which the cells are no longer growth inhibited by antiestrogens (Nicholson, R. I. et al. (2000) Br. J. Cancer 82, 501-513; Clarke, R. et al. (2001) J. Steroid Biochem. Mol. Biol. 76, 71-84; Nicholson, R. I. et al. (2003) Breast Cancer Res. Treat. 80 Suppl 1, S29-34; discussion S35; Clarke, R. et al. (2003) Oncogene 22, 7316-7339). It is certain that endocrine resistance is multi-factorial. Since breast cancers display a remarkable phenotypic heterogeneity as a result of distinct gene expression profiles (Perou, C. M. et al. (2000) Nature 406, 747-752; Sorlie, T. et al. (2001) Proc Natl Acad Sci USA 98, 10869-10874), each cancer type likely utilizes a different resistance mechanism. Nonetheless, aberrations in ER signaling pathways appear to be critical events that drive the response and resistance to antiestrogens. A rise in the population of ER mutants as ligand-independent, constitutively active or dominant-negative phenotypes, is postulated to contribute to the endocrine resistance of tumors (Murphy, L. C. et al. (1997) Ann. Med. 29, 221-234; Leygue, E. et al. (1998) Cancer Res. 58, 3197-3201). Despite the fact that ERα and ERβ possess similar structural and biochemical properties, they display distinct activation properties for the expression of estrogen responsive genes. An alteration in the relative levels of ERα and ERβ when co-synthesized could, therefore, contribute to endocrine resistance by offsetting the balance between the regulatory potentials of ER-subtypes (Lazennec, G. et al. (2001) Endocrinology 142, 4120-4130; Speirs, V. et al. (1999) Cancer Res. 59, 525-528; Speirs, V. et al. (1999) Cancer Res. 59, 5421-5424). Aberrations in signaling pathways converging onto ER (post-translational processing) and/or ER-mediated events (promoter cross-talk) could also contribute to resistance by altering the sensitivity of ligand-ER mediated events or by circumventing the need for ligand-driven cell-growth (Kato, S. et al. (1998) Oncology 55 Suppl 1, 5-10; Nicholson, R. I. et al. (1999) Endocr Relat Cancer 6, 373-387).

Alterations in co-regulator expression or availability could also be one mechanism for the development of endocrine resistance. Tamoxifen resistance is characterized not only by the ineffectiveness of the compound to inhibit tumor growth but also by a gained ability to act as a partial agonist in breast cells. Co-regulatory proteins are present at rate-limiting levels in cells such that modification in the level of co-regulator expression or activity could lead to alterations in the ER signaling, consequently endocrine resistance (Shang, Y. et al. (2002) Science 295, 2465-2468). As discussed above, the transcriptional activity of the tamoxifen-ERα complex is modulated by the ratio between co-activator and co-repressor recruited to the complexes in cells within which tamoxifen acts as a partial agonist (Fujita, T. et al. (2003) J. Biol. Chem. 278, 26704-26714). A decrease in the level or activity of co-repressors (Lavinsky, R. M. et al. (1998) Proc. Natl. Acad. Sci. USA 95, 2920-2925; Graham, J. D. et al. (2000) J. Steroid Biochem. Mol. Biol. 74, 255-259; Graham, J. D. et al. (2000) Steroids 65, 579-584) with or without a concurrent increase in the level of co-activators (Hudelist, G. et al. (2003) Breast Cancer Res. Treat. 78, 193-204; Font de Mora, J. et al. (2000) Mol. Cell. Biol. 20, 5041-5047) could therefore play a critical role in the development of tarnoxifen resistance in ER positive breast cancers.

Studies showed that ERα positive breast cancer cells that are resistant to the growth-inhibitory effects of tamoxifen remain sensitive to growth inhibition by ICI 182,780 in experimental models in situ (Clarke, R. et al. (2001) Pharmacol. Rev. 53, 25-71; Brunner, N. et al. (1993) Cancer Res. 53, 3229-3232). It is likely that the ability of ICI 182,780 to promote monomerization of ER and subsequent degradation by preventing the nuclearicytopiasm shuttling of ER is the basis for its effectiveness as an antiestrogen. This interpretation is also consistent with second-line endocrine responses in patients who had relapsed on tamoxifen but responded to ICI 182,780 (Howell, A. et al. (1996) Br. J. Cancer 74, 300-308). It is unknown whether patients undergoing ICI 182,780 treatment develop resistance to the compound. However, the continuous long-term exposure of estrogen responsive breast cancer cells that are initially growth inhibited by ICI 182,780 develop resistance to the compound (Larsen, S. S. et al. (1997) Int. J. Cancer 72, 1129-1136; Brunner, N. et al. (1997) Cancer Res. 57, 3486-3493), as observed with experimental cell models (for a review see: [Simpson, E. R. et al. (2002) Recent Prog. Horm. Res. 57, 317-338]). This appears, at least in part, to be due to the re-bounding synthesis of ERα with a concomitant increase in responsiveness to estrogens (Larsen, S. S. et al. (1997) Int. J. Cancer 72, 1129-1136). The regulation of ERα gene expression involves activity of several distinct promoters whose activities are mediated by AP-1, AP-2, and estrogen receptor factor 1 (ERF-1) binding sites that interact with a member of the AP-2 family proteins (Tang, Z. et al. (1997) Mol. Cell. Biol. 17, 1274-1280; deConinck, E. C. et al. (1995) Mol. Cell. Biol. 15, 2191-2196; Tanimoto, K. et al. (1999) Nucleic Acids Res. 27, 903-909). Similarly, Alu ERE, Oct-1, AP-1 and SP-1 sites regulate the expression of the ERβ gene (Li, L. C. et al. (2000) Biochem. Biophys. Res. Commun. 275, 682-689). There is evidence that both ERs also auto-regulate their own transcription (Castles, C. G. et al. (1997) J. Steroid Biochem. Mol. Biol. 62, 155-163; Vladusic, E. A. et al. (2000) Oncol Rep 7, 157-167). It is likely that modulation of the synthesis or activity of transacting factors responsible for the ER expression could be responsible for re-bounding/increase expression of ERs. This, together with findings that ICI 182,780 treatment can also lead to cross-resistance to tamoxifen (Brunner, N. et al. (1997) Cancer Res. 57, 3486-3493), indicates that the estrogen-mediated ER signaling participates in the development of acquired endocrine resistance. This reinforces expectations that inhibition of estrogen biosynthesis by aromatase inhibitors or by GnRH analogs together with antiestrogenic compounds could provide more effective treatment regimens for hormone responsive breast cancer.

As in endocrine de novo resistance, growth factor signaling pathways become up-regulated and/or activated in resistant breast cancer cells, which show an increased dependence on growth factor signaling pathways as an adaptive mechanism (Yarden, Y. (2001) Oncology 61 Suppl 2, 1-13). Therefore, blockage or inhibition of growth factor signaling pathways in acquired endocrine resistance could also provide a basis for treatment. Indeed, in situ, in vivo, and clinical studies clearly indicate that inhibition of a variety of growth factor-mediated signaling processes is effective in the prevention of endocrine-resistant phenotypes Nicholson, R. I. et al. (2001) Endocr Relat Cancer 8, 175-182; Jeng, M. H. et al. (2000) Breast Cancer Res. Treat. 62, 167-175). The efficacy of anti-growth factor modalities can be further enhanced by combined treatments involving estrogen synthesis inhibitors and/or antiestrogens (Wakeling, A. E. et al. (2001) Clin Cancer Res 7, 4350s-4355s; discussion 4411s-4412s).

c) Gene Based Approaches for Breast Cancer

Estrogens and growth factor signaling pathways mediate many interdependent events optimal for cell growth and survival. In the absence or loss of regulatory inputs from estrogen signaling due to deficiencies in ER expression/function, aberrations in signaling pathways mediated by growth factors appear to be central to de novo endocrine resistant cancers. Targeted inhibition of the growth factor signaling cascade is a target in the treatment of de novo resistant breast cancers. In addition, induction of ER expression/re-expression could provide a novel therapeutic benefit by restoring functional estrogen signaling. Consistent with this premise, experimental studies have shown that re-introduction of ERα or of ERβ into ER-negative breast cancer cell models by stable transfections or by an adenoviral gene delivery re-establishes some features of estrogen responsiveness, decreases proliferation, motility and invasiveness of tumor cells (Lazennec, G. et al. (2001) Endocrinology 142, 4120-4130; Kushner, P. J. et al. (1990) Mol. Endocrinol. 4, 1465-1473; Garcia, M. et al. (1992) Proc Natl Acad Sci USA 89, 11538-11542; Zajchowski, D. A. et al. (1993) Cancer Res. 53, 5004-5011; Catherino, W. H. et al. (1995) Cancer Lett. 90, 3542; Lazennec, G. et al. (1999) Mol. Cell. Endocrinol. 149, 93-105; Lazennec, G., et al. (1999) Mol. Endocrinol. 13, 969-980). These findings concur the prediction that establishment of functional estrogen-ER signaling could provide an effective treatment strategy for ER-negative breast cancers.

Although, as in de novo endocrine resistance, aberrant growth factor signaling pathways play a critical role in the initiation and progression of acquired endocrine resistance, gain-of-function of ER is coincident with the development of resistance. This implies that additional therapeutic benefits could be achieved by reducing ER function. Strategies to further ablate the intracellular activity of ER by employing anti-sense technologies to inhibit ER expression and ER-dominant negative mutants to interfere with protein function constitute paradigms for the development of novel strategies for breast cancer treatments.

(1) Targeting ER

Dominant-negative receptors are structurally altered forms that are deficient in transcriptional activation of responsive genes, but they retain the ability to suppress the activity of the wild-type receptor (Herskowitz, I. (1987) Nature 329, 219-222). Studies exploring the functional inactivation of ERα have identified and generated ERα mutants with dominant-negative phenotypes. These include truncated or point mutant ERα variants in which substitution or deletion mutations in the carboxyl terminal region severely affect the estrogen-dependent AF-2 function (Ince, B. A. et al. (1993) J. Biol. Chem. 268, 14026-14032). Using adenovirus-mediated gene delivery systems, further studies found that expression of dominant-negative or anti-sense ERα effectively suppresses proliferation and induces apoptosis in both ER-positive breast cancer cells (Lee, E. J. et al. (2001) Mol. Med. 7, 773-782; Lazennec, G., et al. (1999) Mol. Endocrinol. 13, 969-980; Madden, T. A. et al. (2000) Eur. J. Cancer 36 Suppl 4, S34-35) and xenograft models (Lee, E. J. et al. (2001) Mol. Med. 7, 773-782). Dominant negative interference is also the basis for the generation of novel ER proteins. Genetic integration of either the Krupple-associated box (KRAB) (de Haan, G. et al. (2000) J. Biol. Chem. 275, 13493-13501) or the repression domain of NCoR (Chien, P. Y. et al. (1999) Mol. Endocrinol. 13, 2122-2136) as single or multiple copies to WT or mutant ERα produced dominant negative ER species that effectively repressed both basal and estrogen-ER mediated transcription in situ. Similarly, a chimeric receptor composed of the DBD of ERα for binding to ERE, the KRAB repressor domain to silence target genes and a truncated LBD of progesterone receptor to respond only to the synthetic ligand, RU486, effectively blocks estrogen-ER mediated transcription under the control of RU486 (Ma, Z. Q. et al. (1999) J. Steroid Biochem. Mol. Biol. 69, 155-163).

These approaches are confirmatory for the prediction that inhibition of ER synthesis/function could provide an alternative/complementary modality for targeted therapy of breast cancers. Dominant negative interference of ERα is suggested to occur at multiple levels of the signaling pathway that includes the formation of inactive heterodimers, competition for ERE binding, and specific transcriptional silencing. However, determining target gene responses during co-synthesis of different ER species is difficult. This is because dimers formed in the presence of more than one ER species are a heterogeneous population of homo- or hetero-dimers. Moreover, heterodimerization also provides novel characteristics to ER by combining distinct functions of the contributing partners (Tremblay, G. B. (1999) Molecular & Cellular Biology 19, 1919-1927; Muyan, M. et al. (2001) Mol. Cell. Endocrinol. 182, 249-263). This combinatorial diversity, hence heterogeneity, of ERs is an obstacle for the use of variants as effective means of regulating estrogen signaling.

Disclosed herein is a genetic conjugation approach to circumvent the pivotal dimerization step in receptor action (Muyan, M. et al. (2001) Mol. Cell. Endocrinol. 182, 249-263). Homofusion ERα was engineered as a prototype by genetically conjugating two ER monomers into a covalently fused single-chain protein to obtain a homogeneous population. Disclosed herein the single-chain ERα is in a “closed, dimer-like” configuration through an intra-molecular interaction, in contrast to WT-ERα that is a homodimer by an inter-molecular assembly (Muyan, M. et al. (2001) Mol. Cell. Endocrinol. 182, 249-263). The genetic conjugation approach precludes the limitation of monomer association into biologically active dimers and the dissociation of dimers into inactive monomer induced by destabilizing mutations. This method offered an opportunity to expand the understanding of structure-function relationships of not only ER homodimers but also heterodimers with biological activity that is difficult to assess due to presence of the homodimers of contributing partners. Permitting functional analysis of unique symmetrical or asymmetrical mutations that simulate variant homo- and heterodimers, the genetic conjugation can also allow elucidation of the mechanisms of action of specific homo- and hetero-dimers of ER variants. This, in turn, can reveal their contribution to tumor progression and acquisition of resistance to antiestrogen treatments. To examine the effects of a dominant-negative receptor on transcription induced by WT-ERα, transcriptionally defective mutants in which point mutations obliterate the AF-2 function were generated. The WT ERα (ERα) and/or the AF-2 mutant (AF2) were then genetically fused to generate single chain receptors that emulate the WT homodimer (α-α), heterodimer (ERα-AF2 and AF2-ERα) and AF-2 homodimer (AF2-AF2). When co-expressed with WT-ERα, only the single-chain variant deficient in both AF-2 functions repressed the reporter activity more effectively than the ERα variant bearing the same AF-2 mutation, which has the ability to also heterodimerize with WT-ERα (Muyan, M. et al. (2001) Mol. Cell. Endocrinol. 182, 249-263). This single-chain ER approach provides a model for generating fusion constructs to inhibit tumor growth by either deactivation of dominant-positive or activation of dominant-negative pathways.

(2) Targeting EREs.

Dominant negative ER variant or single-chain ER strategies to attenuate or completely prevent ER functions can provide effective approaches to combat breast cancer. Since the interaction of the estrogen-ER complex with ERE of each responsive gene constitutes a critical genomic signaling pathway, targeted regulation (activation/repression) of ERE-driven genes disclosed herein provides a compelling approach in breast cancer treatments.

The disclosed ERE binding transregulators, on the other hand, potently regulate the expression of ERE-containing genes independent of ligand, dimerization, ER-subtypes, promoter- and cell-context without altering ERE-independent genomic signaling. Furthermore, transregulators, just as the estrogen-ERα complex, differentially altered cell cycle progression in cells derived from breast cancer. Disclosed herein ERE binding activators repress the cell cycle progression in ERα-negative MDA-MB-231 cells, while they enhance cell cycle progression in ERα-positive MCF-7 cells (Huang, J. et al. (2004) Mol. Cell. Endocrinol. 218, 65-78).

Emphasizing the importance of ERE-containing genes in the regulation of cell proliferation, these findings indicate that EBAs can be used for identification and subsequent elucidation of the roles of ERE-containing genes in physiology and pathophysiology of breast tissue. This provides a better understanding of the differences in the mechanisms of genomic responses manifested as phenotypic diversity among breast tumors. This understanding can aid strategies for cancer treatment. The suppression of ERE-containing genes, for example, by the designer ERE-binding repressors can constitute a therapeutic approach for ER expressing endocrine sensitive and acquired endocrine resistant breast tumors. Furthermore, the activation of the ERE-containing gene network by EBAs could provide an effective strategy for the treatment of ER-negative neoplasms. This can serve as a basis to alter the growth of breast tumors independent of ER status. Furthermore, in contrast to antiestrogen therapy, such constructs could be targeted to specific tissues of interest by gene therapy approaches.

(3) Establishment of an Effective Gene Delivery System.

Recombinant adenoviruses are versatile tools for gene delivery and expression due to wide cellular tropisms and prolonged transgene expression profiles that are independent of host cell division. The most commonly used adenoviral vector is the human adenovirus serotype 5 (Ad5), which is rendered replication defective by the deletion of the essential genes, E1 and E3. Ad5 has been adapted into an efficient vector for in situ, ex vivo and in vivo gene transfer (Kozarsky, K. F. et al. (1993) Curr. Opin. Genet. Dev. 3, 499-503; Channon, K. M. et al. (1997) QJM 90, 105-109; Young, L. S. et al. (2001) Gut 48, 733-736). Recent studies further indicated that Ad5 could successfully be used for gene transfer into breast cancer cell lines with high efficiency (Lee, E. J. et al. (2001) Mol. Med. 7, 773-782; Lazennec, G. et al. (1999) Mol. Cell. Endocrinol. 149, 93-105; Lazennec, G., et al. (1999) Mol. Endocrinol. 13, 969-980; Lucas, A. et al. (2003) Biochem. Biophys. Res. Commun. 309, 1011-1016). One major advantages of recombinant adenovirus compared to other gene delivery systems is the fact that virus remains epichromosomal, that is not incorporated into the host genome, in cells. This prevents inadvertent activation or inactivation of the host genome by the adenovirus genes.

Disclosed herein is the use of the AdEasy XL system (Stratagene, La Jolla, Calif.) to generate recombinant adenovirus as our gene delivery vehicle. The AdEasy XL system significantly shortens the time required producing the recombinant adenoviral plasmid by homologous recombination in E.coli. The cDNA for β-galactosidase, CDC, ERα, an EBA or EBR was cloned into the shuttle vector, pShuttle-CMV. In this vector, the strong cytomegalovirus (CMV) promoter drives the expression of the gene of interest. The resultant vector was transformed into E.coli strain BJ5183-pAD-1 pre-transformed with the adenoviral backbone plasmid pAdEasy-1 for homologous recombination. Bacterial colonies containing the recombinant adenoviral plasmid were selected against kanamycin, propagated and purified by a DNA Maxi prep kit (Qiagen). The recombinant vector was then linearized and transiently transfected into AD-293 cells for virus production. AD-293 cells are derived from human embryonic kidney cells, HEK 293, which are transformed by the sheared Ad5 DNA. AD-293 cells produce adenovirus in trans allowing the production of infectious virus particles when cells are transfected with pAdEasy-1 vector.

### d) Cell Models for Breast Cancer Phenotypes

Establishment of an effective gene delivery system to breast cancer cells can make our approach more amenable for examining the effects of altered expression of ERE-containing genes. The ER-negative MDA-MB-231 cell line (American Type Culture Collection, ATCC, Rockville, Md.) can be used as a model for de novo endocrine resistant breast cancer phenotype. The use of MCF-7 cells (ATCC) that endogenously synthesize ERs, primarily ERα, can represent the model cell line emulating endocrine sensitive breast neoplasms.

The MCF7/LCC sublines can be used as experimental cell models. The generation of endocrine resistant sublines through in vitro selection of the parent MCF-7 cell line against antiestrogens provided a means to study acquired resistance. These sublines retain ER expression and show various patterns of resistance and cross-resistance to antiestrogens. LCC variants were established from an MCF-7 phenotype that grew independent of E2 in ovariectomized nude mice (Clarke, R. et al. (1989) Proc. Natl. Acad. Sci. USA 86, 3649-3653). Subsequent in vitro propagation of cells from these tumors led to the generation of an MCF7/LCC1 phenotype that retains ER expression and E2-independent growth but are responsive to growth inhibition by Type I and II antiestrogens (Brunner, N. et al. (1993) Cancer Res. 53, 283-290). Although transplanted LCC1 cells efficiently produce tumors in the absence of E2, tumor growth is accelerated in the presence of E2. Therefore, LCC1 phenotype retains some degree of E2 responsiveness in vivo (Brunner, N. et al. (1993) Cancer Res. 53, 283-290). Further selection of MCF7/LCC1 cells with graded concentrations of TAM resulted in a TAM-resistant MCF7/LCC2 variant. This subline also showed E2-independent growth in vitro and in vivo but were sensitive to growth inhibition by ICI (Brunner, N. et al. (1993) Cancer Res. 53, 3229-3232). It is this subline that provides a model for tumors that initially responded to Type I antiestrogens, but then acquired resistance to these compounds (Brunner, N. et al. (1997) Cancer Res. 57, 3486-3493). Subsequent selection of MCF7/LCC1 cells against increasing concentrations of ICI resulted in the phenotype of MCF7/LCC9, which is ER-positive and E2 independent, and ICI and TAM-crossresistant (Brunner, N. et al. (1997) Cancer Res. 57, 3486-3493). This subtype is a model for a full endocrine resistant breast cancer. LCC sublines are reported to be more invasive and metastatic in vivo than parental MCF-7 cells (Clarke, R. et al. (2001) Pharmacol. Rev. 53, 25-71). It appears that LCC phenotypes up-regulate some E2 responsive genes, exemplified by the pS2 and PR genes, with concurrent loss in responsiveness to the ligand-ER complex (Brunner, N. et al. (1993) Cancer Res. 53, 283-290; Gu, Z. et al. (2002) Cancer Res. 62,3428-3437). MCF7/LCC1, /LCC2 and /LCC9 cell lines were obtained and are described in Clarke R. et al. (2001) Pharm. Rev. 53:25-71.

C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular (VP16)₂-CDC-(VP16)₂ is disclosed and discussed and a number of modifications that can be made to a number of molecules including the (VP16)₂-CDC-(VP16)₂ are discussed, specifically contemplated is each and every combination and permutation of (VP16)₂-CDC-(VP16)₂ and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Disclosed herein are transactivators that specifically target ERE to regulate the expression of ERE-containing genes. The modular nature of ER permitted the design of a monomeric ERE binding module by genetically joining two DNA binding domains with the hinge domain. Disclosed herein is the integration into this module of strong activation domains from other transcription factors to generate constitutively active ERE binding activators (EBAs). EBAs potently induced the expression of only ERE-containing genes independent of ligand, dimerization, ER subtypes, promoter- and cell-context. Moreover, EBAs, as E2-ERα, differentially altered cell cycle progression in cells derived from breast cancer.

1. Linked Modulators

As discussed herein, linked modulators are engineered molecules that bind DNA at specific sites. Linked modulators can act as transcription modulators, by for example, activating transcription or repressing transcription. Linked modulators comprise at least two DNA binding domains as discussed herein, which are covalently linked. The covalent linkage occurs via a linker, which in certain embodiments can be a hinge as discussed herein. The linked modulators can include a number of other domains, such as activation domains or repressor domains, or other interaction domains, or nuclear localization signals, or targeting domains for example degradation, or regulation domains, such as domains that regulate them depending on the type of cell the linked modulator is in.

One way of representing linked modulators is by using the formula W-Z-X, wherein W is a first DNA binding domain (DBD), X is a second DNA binding domain, and Z is a linker. The linked modulator can further comprise an activation domain or repressor domain designated Y. Disclosed are any permutations of these combinations. It is understood that the two DNA binding domains can be the same or similar or different domains. In addition, as disclosed herein there can be more than one activation or repressor domain. These can be designated as Y₁, Y₂, Y₃, Y₄, Y₅, Y₆, and so forth. Thus, the linked modulators, for example, could be Y₁,-W-Z-X or Y₁-Y₂-W-Z-X- Y₃, and so forth. It is understood that when there is more than one activation or repressor domain, the domains can be the same or different domains, for example, two VP16 domains, or one VP16 and one p65 domain. It is understood that the activation domains or repressor domains can be fused to the linked modulators at either the amino terminal end or carboxyl end or both of the linked modulator. Furthermore the activation or repressor domains themselves can be linked in either an amino terminal or carboxyl orientation. Furthermore, there can be any number of activation or repressor domains linked in any fashion. For example, there can be one, two, three, four, five, six, seven, eight, nine, ten, or more domains linked on either or both the amino and carboxyl sides of the linked modulator.

It is also understood that proteins have an amino and carboxyl end, and unless indicated otherwise, they are written left to right, amino to carboxyl. Thus, W-X-Y unless otherwise indicated would be “amino terminal-W-X-Y-carboxyl terminal.”

In certain embodiments of the linked modulators which are based on the ER, it is understood that herein the DNA binding domain is referred to as C and the linker or hinge domain is referred to as D. Thus, for ER based linked modulators, a linked modulator could be CDC.

It is also understood that more than one linked modulator can be used. In these embodiments, there would be more than one element that the linked modulator would recognize, on the target DNA. Thus, in this type of embodiment, there would be at least four DNA binding sites, that would be bound by the at least four DNA binding domains, two in each linked modulator, if there are two linked modulators being used. As disclosed herein, the effect of having more than one linked modulator can be synergistic.

Furthermore, a linked modulator could have more than two DNA binding domains, such as three or four DNA binding domains. These types of modulators would also have additional linker regions, and typically would have one less linker than the number of DNA binding domains. Thus, linked modulators, such as W-Z-X-Z-W-Z-X, or any combination are disclosed. It is understood that the DNA binding domains can be the same or different when there are multiple domains.

2. DNA Binding Domains

The DNA binding domain (DBD) is responsible for targeting the receptors to their hormone response elements (HRE). The DNA binding domain binds as a dimer with each monomer recognizing specific contacts in the major groove of the DNA at each half-site. These contacts allow the dimer to read the sequence, spacing and orientation of the half-sites within its response element, and thus discriminate between sequences. Such domains are well-known in the art and can be acquired from many different proteins or enzymes. It is understood that the disclosed compositions can be practiced with any such domain including those not specifically discussed herein. Thus, in certain embodiments compositions comprising a first and a second DNA binding domain, wherein the first DNA binding domain comprises a DNA binding domain of a transcription factor, are disclosed. In certain embodiments the DNA binding domain can be a DNA binding domain from a steroid receptor. For example, the DNA binding domain can be from, for example, Glucocorticoid receptor (GR), Androgen receptor (AR), Progesterone receptor (PR), Thyroid hormone receptor (TR), Retinoid X receptor (RXR), all-trans Retinoic Acid receptor (RARα), and Vitamin D receptor (VDR). Representative examples of the sequence of these proteins can be found in SEQ ID NOs: 53, 57, 61, 65, 69, 73, and 77 respectively. Also disclosed are compositions wherein the DBD is from Estrogen Receptor ERα or ERβ which are set forth in SEQ ID NO: 45 and 49 respectively.

The DNA binding regions of hormone receptors contain two zinc finger-like modules that fold to form a single functional domain. The protein dimer contacts nucleotides in adjacent major grooves on one face of the DNA helix containing a response element. Distinct residues in a region of the first zinc-finger module of the C domain, P-box, determine the DNA binding specificity (sequence discrimination) through a protein-DNA interaction. Whereas residues in the second zinc-finger-like module, the D-box, are involved in the discrimination of half-site spacing through a protein-protein interaction (dimerization) that influence the spacing and relative orientation of the two DNA associated monomers (Freedmnan, L. P. (1992) Endocr Rev 13, 129-145; Mangelsdorf, D.J. et al. (1995) Cell 83, 835-839). These structural constraints prevent each hormone receptor from binding promiscuously to REs by the recognition of the central spacing, sequence and arrangement of half-sites (Freedmnan, L. P. (1992) Endocr Rev 13, 129-145; Mangelsdorf, D.J. et al. (1995) Cell 83, 835-839).

The compositions can comprise a second DBD. Therefore, disclosed are compositions, wherein the second DNA binding domain comprises a-DNA binding domain of a transcription factor. Specifically disclosed are compositions comprising a second DBD from a transcription factor, wherein the transcription factor can be selected from the group of transcription factors consisting of Estrogen receptor (ER), Glucocorticoid receptor (GR), Androgen receptor (AR), Progesterone receptor (PR), Thyroid receptor (TR), Retinoid X receptor (RXR), all-trans Retinoic Acid receptor (RAR), and Vitamin D receptor (VDR). Also disclosed are compositions wherein the DBD is from ERα or ERβ.

The DNA binding domain of the ER can be found at for example SEQ ID NO: 23 (ERα) which is amino acids 182-251 of SEQ ID NO:45 and SEQ ID NO: 25 (ERβ) which is amino acids 144-214 of SEQ ID NO:49, of the GR at SEQ ID NO: 27 which is amino acids 417-491 of SEQ ID NO:53, of the AR at SEQ ID NO: 29 which is amino acids 370-442 of SEQ ID NO:57, of the PR at SEQ ID NO: 31 which is amino acids 562-635 of SEQ ID NO:61, of the TR at SEQ ID NO: 33 which is amino acids 98-170 of SEQ ID NO:65, of the RXR at SEQ ID NO: 35 which is amino acids 131-201 of SEQ ID NO:69, of the RAR at SEQ ID NO: 37 which is amino acids 84-156 of SEQ ID NO:73, and of the VDR at SEQ ID NO: 39 which is amino acids 22-92 of SEQ ID NO:77.

It is understood that the DNA binding domains based on the disclosed steroid receptor proteins can vary, and still maintain their specificity and function. For example, Glucocorticoid (GR), Progesterone (PR) and androgen (AR) receptors recognize hormone response elements (REs) having half-sites, 5′-AGAACA-3′, similar to that of the ERE. Thyroid (TR), retinoid X (RXR), all-trans retinoic acid (RAR) and vitamin D (VD) receptors, on the other hand, interact with REs with half-sites, 5′-AGGTCA-3′, identical to that of ERE. These sites however are arranged in direct (RXRE, RARE, VDRE, TRE) or inverted (GR, PR, AR RE) repeats with various spacing. ERE half-sites without spacing also produce a palindromic TRE. The DNA binding regions of these hormone receptors contains two zinc finger-like modules that fold to form a single functional domain. The protein dimer contacts nucleotides in adjacent major grooves on one face of the DNA helix containing a response element. Distinct residues in a region of the first zinc-finger module of the C domain, P-box, determine the DNA binding specificity (sequence discrimination) through a protein-DNA interaction. The P box of ERα and ERβ contains EGckA, ie. Glu-Gly-Cys-Lys-Ala,; whereas the P box of PR, for example,contains GSckV, ie. Glu-Ser-Cys-Lys-Val amino acid sequence. These amino acid composition is critical to the ability of a DBD to discriminate between the PRE/GRE and the ERE. Any mutational change in the P Box sequence of a steroid/thyroid hormone receptor disturbs the specificity of recognition of a DNA response element. When the first amino acid Gly, G in the GR-DBD, for example, was mutated to Trp, W, results in promiscuous binding to many response elements including the PRE/GRE and the ERE. Residues in the second zinc-finger-like module, the D-box, are involved in the discrimination of half-site spacing through a protein-protein interaction (dimerization) that influence the spacing and relative orientation of the two DNA associated monomers. These structural constraints prevent each hormone receptor from binding promiscuously to REs by the recognition of the central spacing, sequence and arrangement of half-sites.

3. Activation Domains

The activation domains that can be attached and used as disclosed herein can in certain embodiments be any activation domain. By activation domain is meant a protein domain that when brought within proximity of a particular transcription start site, will cause transcription from that site to be increased relative to transcription from that site in the absence of the activation domain. Such modulation can represent an activation of gene expression. Herein, “activation of expression” is understood to mean any increase in the transcription expression of a particular gene above the native amount or above a control amount. For example, if a particular composition comprising a transcription modulator or linked modulator is used to change gene expression and the modulator is an activation domain, then “activation” would be any increase in the gene expression with the activation domain over the gene expression of a control without the transcriptional modulator.

Activation domains can be amphipathic helices (containing polar and nonpolar regions) or aliphatic regions of peptide (containing uncharged, nonpolar hydrocarbon residues). Activation domains can also be regions of peptide that have a strong acidic nature because of the presence of acidic residues, which are called acidic activation domains. One example of an activation domain is the strong AD of the herpes simplex virion protein, VP16. This is an acidic activation domain. Another example of an AD is the p65 subunit of the nuclear factor κB, NFXκB. An example of a VP16 AD can be found in SEQ ID NO: 42, and an example of a p65 AD can be found in SEQ ID NO:41. Other examples of activation domains include, but are not limited to the human heat shock factor HSF-1 and GAL4. It is understood and and herein contemplated that activation domains can comprise any activation domain known or unknown in the art. Thus, one embodiment is an acidic activation domain. Another embodiment is an aliphatic activation domain. Also disclosed is an amphipathic activation domain. It is understood that more than one activation domain may be present. Therefore, specifically contemplated are multiple activation domains. For example, specifically disclosed are compositions comprising one, two, three, four, six, eight, and ten activation domains. It is understood that activation domains from different sources can be used to obtain optimal modulation of transcription. Therefore, specifically disclosed are compositions comprising an activation domain, wherein the activation domain comprises one VP16 activation domain and one p65 activation domain. Thus, it is understood that the disclosed compositions can comprise one amphipathic and one acidic activation domain. Also disclosed are compositions comprising multiple acidic and/or multiple amphipathic activation domains. Thus specifically contemplated and disclosed are compositions, wherein the activation domain comprises two VP 16 activation domains and two p65 activation domains.

The activation domains can be characterized by how much they increase transcription. Disclosed are activation domains that, when used with linked modulators, increase transcription at least 2, 3, 4, 5, 6, 7, 8, 9, 10, 15, 20, 25, 30, 50, 100, 150, 200, 500, or 1000 fold relative to no linked modulator and activation domain. Furthermore, in certain situations, activation can be considered additive, meaning, for example, the activity of one domain is X, the activity of two domains is 2X, the activity of three domains is 3X, and the activity of four domains is 4X, and so forth. Another type of activity is synergistic activity, which means that the total effect of the activation is more than what the additive effect would be. For example, if activity of one domain is X, and the activity of four domains is, for example, 10X (more than 4X), the activity would be synergistic.

4. Repressor Domains

Similarly, the linked modulators can comprise repressor domains of gene expression. By repressor domain is meant a protein domain that when brought within proximity of a particular transcription start site, will cause transcription from that site to be decreased relative to transcription from that site in the absence of the repressor domain. Such modulation can represent an repression of gene expression. Herein “repression of expression” refers to any decrease in the level of gene expression below the native or a control amount. For example, if a particular composition comprising a transcription modulator is used to change gene expression and the modulator is a repressor domain, then “repression” would be any decrease in the gene expression with the repressor domain below the gene expression of a control without the transcriptional modulator. The repressor domains can be characterized by how much they decrease transcription. Disclosed are repressor domains that, when used with linked modulators, decrease transcription at least 2, 3, 5, 6, 7, 8, 9, 10, 15, 20, 25, 30, 50, 100, 150, 200, 500, or 1000 fold relative to no linked modulator and repressor domain.

Examples of repressor domains include, but are not limited to Kripple associated box (KRAB) and Sin3 interaction domain (SID). Other examples of repressor domains include, but are not limited to MAD, DAD, and ChromoShadow. It is also understood that more than one transcriptional modulator or repressor domain can be used in the compositions described herein. Thus specifically disclosed are compositions comprising a repressor domain, wherein the repressor domain comprises one, two, three, four, five, six, seven, eight, nine, ten or more activation domains, such as a KRAB repressor domain or a SID repressor domain. It is understood that activation domains from different sources can be used to obtain optimal modulation of transcription. Therefore, specifically disclosed are compositions comprising a repressor domain, wherein the repressor domain comprises one KRAB repressor domain and one SID repressor domain. Also disclosed are compositions, wherein the repressor domain comprises two KRAB repressor domains and two SID repressor domains. An example of a KRAB repressor domain can be found in SEQ ID NO:44. An example of a SID repressor domain can be found in SEQ ID NO:43.

The Kruppel associated box (KRAB) domain is an amino acid sequence motif found at the amino-terminus of nearly one-third of all Krüppel/TFIIIA-type C2H2 zinc finger proteins (Bellefroid E J. et al. (1991) Proc Natl Acad Sci USA 88:3608-12). This highly conserved domain displays potent, DNA-binding dependent repression of transcription that requires the KRAB-associated protein-1 (KAP-1) as the co-repressor (Margolin J F et al. (1994) Proc Natl Acad Sci USA 91:4509-13; Friedman J R et al. (1996) Genes Dev 10:2067-78; Moosmann P. et al. (1996) Nucleic Acids Res 24:4859-67;

Moosmann P. et al. (1997) Biol Chem 378:669-77). The KAP-1 co-repressor in turn recruits the nucleosome remodeling histone deacetylase complex (NuRD). The NuRD complex is composed of three functionally distinct proteins, Mi-2, MTA-2 and MBD3 that assemble through multiple inter-molecular interactions to form the complex. Mi-2 possesses an ATP-dependent chromatin remodeling activity and anchors the NuRD complex to the core HDAC1 and HDAC2 complexes that deacetylate histones. The methyl-DNA-binding protein MBD3, on the other hand, appears to mediate the ability of NuRD to repress the transcription in a methylation-dependent manner. The subsequent interaction of the NuRD-HADC complex with HP1, a family of nonhistone heterochromatin-associated proteins, results in the formation of a heterochromatin-like complex, leading to gene silencing. All members of the KRAB domains repress transcription when tethered to DNA. Fusions of the KRAB domain derived from KOX1/ZNF10 zinc finger protein to the DNA binding domains of both LexA and Gal4 factors, for example, effectively silence the transcription of reporter genes in transfected cells regardless of the distance of the cognate binding element to the transcription initiation site. In addition to the recruitment of co-repressor complexes by the fusion proteins to the promoter, specific inhibition of some component(s) of RNA polymerase II and III transcription by KRAB appears also involved in transcription suppression (Moosmann P. et al. (1996) Nucleic Acids Res 24:4859-67; Moosmann P. et al. (1997) Biol Chem 378:669-77).

Transcriptional repression by the basic helix-loop-helix zipper protein Mad1 requires DNA binding as a ternary complex with its heterodimer partner Max and the mammalian co-repressor protein Sin3. Similarly, the repression of thyroid hormone responsive genes is mediated by the recruitment of Sin3 repressor complex by the unliganded TR-corepressor N-CoR/SMRT complexes. The Sin3 complex contains two additional proteins, SAP18 and SAP30. These proteins are integral components of the Sin3 complex. They are involved in the facilitation or stabilization of co-repressor interactions with Sin3 and HDACs. The interaction between Madl and Sin3 is mediated by a 35 amino-acid region in the amino-terminus of Madl (Sin3 interaction domain, or SED) (Kasten MM. et al. (1996) Mol Cell Biol 16:4215-21, AyerDE et al. (1996) Mol Cell Biol 16:5772-81.

The tethering of SID, just as KRAB, to a DBD of a transcription factor suppresses gene transcription mediated by the response element to which the parent transcription factor binds. As NuRD complex, Sin3 or SID-mediated transcriptional repression involves HDAC1 and HDAC2 recruitment to the promoter. Thus, although a distinct set of transcriptional repressors recruits different co-repressor complexes to establish a repressor, the recruitment of HADC1 and HDAC2 by NuRD or Sin3 complex is critical for the active gene repression (Kasten M M. et al. (1996) Mol Cell Biol 16:4215-21, Ayer DE et al. (1996) Mol Cell Biol 16:5772-81). In short, genetic conjugation of a single or multiple copies of SID, or KRAB, to either terminus or both termini of CDC provides a potent ERE-binding transrepressor. This is because SID or KRAB recruits correpressor proteins to the gene the CDC binds. This complex then recruits HDACs to lead to transcription repression.

It is understood that it is possible for the compositions to a DBD linked to a KRAB repressor, wherein the DBD is from c-Myb (MybDBD SEQ ID NO: 82). It is an embodiment that the composition excludes a DBD from c-Myb or KRAB or linkages thereof For example, one embodiment excludes MybDBD-KRAB and one embodiment does not consist of MybDBD-KRAB (Nawrath, M. et al. (2000) Cancer Gene Ther 7(6), 963-972). Nawrath et al. is herein incorporated by reference in its entirety for its teachings of MybDBD-KRAB.

It is also understood that in certain embodiments a linked modulator, comprising two DNA binding domains connected by a linker, can act as a repressor. In these embodiments, no repressor domain is needed to for the linked modulator to function as a repressor. This is consistent with the linked modulator competing with an endogenous transactivator.

Furthermore, it is an embodiment that one or more of the components of the composition or methods disclosed herein can be specifically excluded.

5. Linker and Hinge Regions

The DNA binding domains of the linked modulator can be any linker. Typically they will be a peptide linker. Often they will be a peptide linker that is or has homology to a hinge region of a steroid receptor, such as the ER. Hinge domains are well known in the art of receptors and hormone response elements. Hinge domains (also referred to herein as linker domains) are herein contemplated to mean a flexible region comprising a nuclear localization signal which allows for transactivators to localize to the nucleus and functions to link two separate DNA binding domains yet providing flexibility to allow efficient interaction with a response element. The importance of flexibility of the linker domain is evident via the poor binding of DBD linked by the hinge domain of SEQ ID NOs: 83 and 84 (Kuntz, M. A. et al. (1997) J Biol Chem 272, 27949-279456). The lack of flexibility in the linker domain, did not allow for efficient binding of the DBD to an ERE. The resulting binding produced a dissociation constant (K_{D}) of 38 nM. More flexible linkers allow for stronger binding. For example, two ERα C domains linked by a ERα D domain (CDC) has a K_{D}=1.49±0.16 nM for binding to ERE. Thus, one embodiment is a linker, wherein the linker does not comprise SEQ ID NO: 83 or 84. Linker domains can be derived from receptors where the hinge domains link domains in the receptor. For example, it is understood that the hinge domain can comprise an ER D-domain. The D-domain can comprise any one of SEQ ID NOs:24, 26, 28, 30, 32, 34, 36, 38, or 40. It is understood that the hinge domain can comprise the ER D-domain of ERα or ERβ, and can comprise SEQ ID No 24 or SEQ ID NO:26, respectively. It is also understood that hinge domains from other hormone receptors can be used with equal effectiveness

The hinge domain of the GR at SEQ ID NO:28, of the AR at SEQ ID NO:30, of the PR at SEQ ID NO:32, of the TR at SEQ ID NO:34, of the RXR at SEQ ID NO:36, of the RAR at SEQ ID NO:38, and of the VDR at SEQ ID NO:40.

6. Examples of Linked Modulators

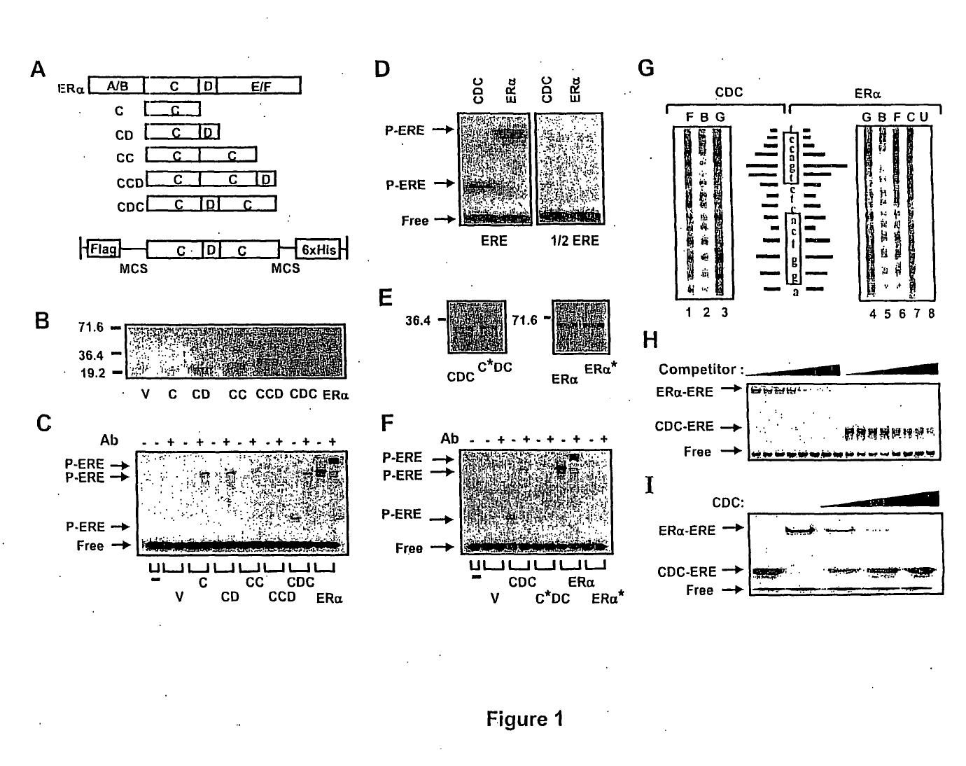

One example of linked modulators are proteins based on ERE binding proteins. ERE binding proteins were engineered as potent activators of ERE-containing genes independent of ligand, ER subtypes, dimerization, promoter- and cell-type. Proteins with various configurations were initially generated to obtain an ERE binding module that interacts efficiently with the consensus ERE. As discussed herein, ER recognizes an ERE as a dimer with interaction primarily mediated by a dimerization interface located in the ligand binding domain and through a weak interaction surface in the C domain (Parker, M. G. (1998) Biochem Soc Symp 63, 45-50, Hall, J. M. et al. (2001) J Biol Chem 276, 36869-36872). Each C domain of the ER dimer makes analogous contacts with one of the inverted motifs, resulting in a rotationally symmetric structure (Glass, C. K. (1994) Endocr Rev 15, 391-407; Luisi, B. F.et al. (1994) Vitam Horm 49, 1-47). Although the isolated C domain can interact with a half-site of the consensus ERE as a monomer, albeit with a substantially lower DNA binding affinity than that of the parent receptor, it requires ERE-dependent dimerization for the formation of a stable C-ERE complex (Schwabe, J. W. et al. (1993) Cell 75, 567-578; Nardulli, A. M. et al. (1991 J Biol Chem 266, 24070-24076; Kuntz, M. A. et al. (1997) J Biol Chem 272, 27949-279456). ERs share 97% amino-acid identity in their DBDs (Mosselman, S.et al. (1996) FEBS Letters 392, 49-53). This is reflected in their abilities to bind various ERE sequences with similar specificity and affinity by interacting with the same nucleosides (Yi, P. et al. (2002) Mol Endocrinol 16, 674-693). Thus, the C domain of ERα was chosen as the template protein in the engineering of ER subtype-independent ERE binders (SEQ ID NO:23). The engineered proteins, linked modulators consisted of two C domains of ERα co-joined without (CC and CCD) or with the D domain as a linker (CDC) by the genetic conjugation approach (FIG. 1A) (Muyan, M. et al. (2001) Mol Cell-Endocrinol 182, 249-263). The choice of the D domain as a linker was based on the following observations. First, although the isolated C domain is the minimal region for ERE binding, the immediate amino-terminal residues of the D domain contribute significantly to the DNA binding affinity by creating a stable interaction between the half-site-bound monomers (Green, S. et al. (1986) Cold Spring Harbor Symposia on Quantitative Biology 51, 751-758; Schwabe, J. W. et al. (1993) Cell 75, 567-578; Kumar, V. et al. (1987) Cell 51, 941-951; Mader, S. et al. (1993) Nucleic Acids Res 21, 1125-1132). Second, the D domain, providing flexibility between the amino- and carboxyl-termini of the receptor, also contains patches of amino-acids that act cooperatively for the nuclear localization of ER (Picard, D. et al. (1990) Cell Regulation 1, 291-299).

7. Steroid Hormone Receptors

Different sets of genes can be regulated by the same transcription modulator. Furthermore, there can be families of transcription modulators which are related in the way that they function or in the types of DNA binding sites they interact with, for example. One family of transcription modulators is the steroid hormone receptors. A steroid hormone receptor generally is a transcriptional modulator that binds DNA in a homo or hetero dimer fashion, and which also binds a small ligand, such as a steroid hormone. Typically, the function of the steroid hormone receptor is to activate transcription, and this function is controlled by the binding of the ligand, the steroid hormone, to the steroid hormone receptor. The binding of the ligand can facilitate dimerization, i.e. in the absence of the ligand, dimerization does not take place, or the ligand can facilitate the effect on transcription by, for example, allowing the appropriate conformation of the activation domain, so that the activation domain can exert its effect on the basal transcription machinery. There are many different members of the steroid hormone receptor of transcription modulators. For example, some members of the steroid hormone receptor family are the Estrogen receptor (ER), Glucocorticoid receptor (GR), Androgen receptor (AR), Progesterone receptor (PR), Thyroid receptor (TR), Retinoid X receptor (RXR), all-trans Retinoic Acid receptor (RAR), and Vitamin D receptor (VDR). One steroid hormone receptor that is used as an example herein is the estrogen receptor.

a) The Estrogen Receptor

The estrogen receptor (ER) can represent an example of other steroid receptors, which might have different specifics but will share the general features discussed herein. The homeodynamic regulation of target tissue function by estrogen hormones, particularly estradiol 17β (E2), is mediated through a complex array of convergent and divergent signaling pathways. These pathways orchestrate genomic and non-genomic events that result in target tissue-specific responses (Parker, M. G. (I 998) Biochem Soc Symp 63, 45-50; McKenna, N. J. et al. (1999) Endocr Rev 20, 321-344; Hall, J. M. et al. (2001) J Biol Chem 276, 36869-36872). The flow of E2 information necessary for genomic effects is encoded in the regulatory DNA elements of each responsive gene and is conveyed by estrogen receptor (ER) α or β as homodimers, or as the heterodimer when co-expressed (Parker, M. G. (1998) Biochem Soc Symp 63, 45-50; McKenna, N. J. et al. (1999) Endocr Rev 20, 321-344; Hall, J. M. et al. (2001) J Biol Chem 276, 36869-36872). As with other transcription factors, ERs are modular such that isolated structural domains display subsets of the functional activities of the intact receptor (Parker, M. G. (1998) Biochem Soc Symp 63, 45-50; McKenna, N. J. et al. (1999) Endocr Rev 20, 321-344; Hall, J. M. et al. (2001) J Biol Chem 276, 36869-36872). The amino-terminus A/B domain contains a ligand-independent transactivation function (AF-1). The central region is the DNA binding domain (DBD), or the C domain. The flexible hinge domain, the D domain, contains a nuclear localization signal and links the C domain to the multi-functional carboxyl-terminus, the E/F domain. E/F mediates ligand binding, dimerization, and the ligand-dependent transactivation function (AF-2) of the receptor.

The elements of the genomic action of ER involve a multi-step regulation in which ER is converted from an inactive form to a transcriptionally active state (Parker, M. G. (1998) Biochem Soc Symp 63, 45-50; McKenna, N. J. et al. (1999) Endocr Rev 20, 321-344; Hall, J. M. et al. (2001) J Biol Chem 276, 36869-36872). This regulation is initiated by a conformational change in ER upon E2 binding, dissociation from chaperone proteins and dimerization. The dimer ER interacts with an element which is an inverted repeat that is separated by three non-specific nucleotides. The consensus estrogen responsive element (ERE) is 5′-GGTCAnnnTGACC-3′ and sequences with one or more nucleotide variations of the consensus can also act as EREs. The binding of ER to an ERE is the initial step in the regulation of ERE-containing gene transcription. Other proteins can also be involved, for example, the ERE bound E2-ERα complex (ERα is a type of ER) interacts with co-regulatory proteins. These additional interactions promote chromatin remodeling and bridging with general transcription factors that result in the formation of a transcriptome. This is a ligand- and ERE-dependent ER signaling pathway.

The E2-ER complex also modulates gene expression by tethering to a trans-acting factor bound to a cis-element, e.g. the AP-1 element-bound fos/jun complexes. These pathways can be called ligand-dependent ERE-independent pathways (Hall, J. M. et al. (2001) J Biol Chem 276, 36869-36872; (Kushner, P. J. et al. (2000) J Steroid Biochem Mol Biol 74, 311-317; Safe, S. (2001) Vitam Horm 62, 231-252). In these contexts, the E2-ER complex is not binding the DNA at its cognate element. In addition, E2 can elicit non-genomic effects through membrane ER or ER isoforms (Razandi, M. et al. (2000) Mol Endocrinol 14, 1434-1447; Gu, Q. et al. (I999) Endocrinology 140, 660-666; Benten, W. P. et al. (2001) Endocrinology 142, 1669-1677) and through the cytoplasmic ER (Chen, Z. et al. (1999) J Clin Invest 103, 401-406; Simoncini, T. et al. (2000) Nature 407, 538-541). These non-genomic pathways are linked to intracellular signal transduction proteins.

Although ER plays a central role in the modulation of ERE-containing genes, the direction and magnitude of gene expression are dictated by the integrated actions of many trans-acting factors (promoter cross-talk), co-activators/integrators, components of the basal transcription machinery and the chromatin within which the gene resides (Parker, M. G. (1998) Biochem Soc Symp 63,45-50; McKenna, N. J. et al. (1999) Endocr Rev 20, 321-344; Hall, J. M. et al. (2001) J Biol Chem 276, 36869-36872). The type and availability of protein components of a transcription complex specific to each gene characterize promoter-and cell-specific responses (Parker, M. G. (1998) Biochem Soc Symp 63, 45-50; McKenna, N. J. et al. (1999) Endocr Rev 20, 321-344; Hall, J. M. et al. (2001) J Biol Chem 276, 36869-36872). These proteins, responding to diverse signaling pathways, act cooperatively or antagonistically with ER to modulate the gene expression (McKenna, N. J. et al. (1999) Endocr Rev 20, 321-344; Hall, J. M. et al. (2001) J Biol Chem 276, 36869-36872; Safe, S. (2001) Vitam Horm 62, 231-252). In addition to the gene promoter, ER and ER associated co-regulatory proteins are targets for various signaling pathways. Post-translational modifications of un-liganded or liganded ER and/or co-regulatory proteins by various signaling pathways alters the ability of the receptor to modulate transactivation of responsive genes (Ali, S. et al. (1993) Embo J 12, 1153-1160; Aronica, S. M. et al. (1993) Mol Endocrinol 7, 743-752; Kato, S. et al. (1995) Science 270, 1491-1494; Nilsson, S. et al. (2001) Physiol Rev 81, 1535-1565; Tremblay, A. et al. (1999) Mol Cell 3, 513-519).

Along with the development and function of the mammary gland as one of the estrogen target tissues, E2 is also involved in the initiation and progression of breast cancer that results from uncontrolled growth and division of mammary ductal epithelial cells (Jensen, E. V. (1996) Annals of the New York Academy of Sciences 784, 1-17; Jordan, V. C. et al. (1999) Endocr Rev 20, 253-278; Russo, J. et al. et al. (2000) J Natl Cancer Inst Monogr, 17-37). The understanding of mechanisms of ER actions, dissection of ER pathways and associated factors in the physiology and pathophysiology of ER signaling are critical prognostic and therapeutic goals in cancer research. The current paradigms to achieve these goals are largely based on compounds with diverse pharmacology that target ER (Jensen, E. V. (1996) Annals of the New York Academy of Sciences 784, 1-17; Jordan, V. C. et al. (1999) Endocr Rev 20, 253-278; McDonnell, D. P. et al. (1999) Trends Endocrinol Metab 10, 301-311). The binding of these ER-ligands to ER induce conformational changes that alter the ability of receptor to interact with co-regulatory proteins whose compositions dictate the receptor activity. However, since ERs are also a target for multiple signals emanating from intracellular signaling pathways (Hall, J. M. et al. (2001) J Biol Chem 276, 36869-36872; (Clarke, R. et al. (2001) J Steroid Biochem Mol Biol 76, 71-84), post-translational processing of ERs could preclude the need of ERs for ligand or could differentially alter the function of the liganded ERs. Furthermore, the circumvention of ligand-ER mediated DNA-dependent events by aberrant signaling pathways that promoter cross-talk with the ligand-ER complex (Hall, J. M. et al. (2001) J Biol Chem 276, 36869-36872; (Clarke, R. et al. (2001) J Steroid Biochem Mol Biol 76, 71-84) would hinder the regulatory effects of ligands. The presence of mutant ERs (Fuqua, S. A. et al. (1993) J Cell Biochem 51, 135-139) that do not rely on ligands for flimction (i.e., dominant-negative or -positive ER variants) or the absence of ER expression (ER negativity) would also render the ligand-based approaches ineffective.

8. Elements and DNA Binding Sites

The DNA binding sites are as discussed herein. DNA binding sites are regions of DNA which are recognized by DNA binding domains. The DNA binding sites can be any site that is recognized by one of the disclosed DNA binding domains. For example, the DNA binding sites can be inverted repeats and direct repeats. When a region of DNA is recognized by a modulator or a dimer of modulators it can be referred to as an element, such as an enhance element.

When an element comprises two DNA binding sites, each site can be referred to as a half site, i.e. each site binds one dimer and there are two monomers in a dimer, each of which binds one half site. Furthermore, elements have two things in common, they all. involve an orientation of the DNA binding sites relative to one another, and they all involve a spacing of the binding sites relative to one another. The orientation of binding sites can be referred to a direct or inverted, and this refers to the orientation of the DNA binding sites relative to one another and relative to how they typically are found in the DNA. For example, generically a half site can be referred to a 5′-A-B-C-3′ where A, B, and C are specific nucleotides. An element that has a direct orientation would be one where each DNA binding site has the same orientation 5′ to 3′. For example, half site one is 5′-A-B-C-3′ and half site two is 5′-A-B-C-3′. An element that has an inverted orientation is one where the second DNA binding site is reversed relative to the first DNA binding site. For example, half site one is 5′-A-B-C-3′ and half site two is 5′-C-B-A-3′. In the preceding examples, each DNA binding site of the element is based on the same DNA binding site, indicating that the dimer which would bind this element would typically be a homodimer. As indicated dimers can be heterodimers, which are formed by different monomers, which would typically have different DNA binding sites. The orientation of the hetero sites can also be direct or inverted, but would be understood relative to the typical orientation of each site in DNA. For example, the first DNA binding site can generically be represented as 5′-A-B-C-3′ and the second DNA binding site can be generically referred to as 5′-D-E-F-3′. If these sites are typically found in this position in DNA then an element oriented as 5′-A-B-C-3′ and 5′-D-E-F-3′ would be a direct element and an element oriented as 5′ A-B-C-3′ and 5′-F-E-D-3′ would be an inverted element. It is clear that there are as many combinations of DNA binding sites forming elements as there are combinations of dimers and multimers (more than two monomers interacting together to bind DNA), and the orientation of these sites can be any permutation. Those of skill in the art understand how to identify DNA binding sites, elements, and the orientation of these sites.

The other common aspect of elements is the spacing between the DNA binding sites. This spacing defines how far the DNA binding sites are from one another. For example, given the DNA binding site, 5′ A-B-C-3′ the element might be 5′-A-B-C-x-x-x-C-B-A-3′. This element has an inverted orientation and a spacing of three, because there are three units, or nucleotide for example, between the half sites.

Examples of Estrogen response elements (ERE) are present in the human pS2 (Nunez, A. M. et al. (1989) EMBO J 8, 823-829), oxytocin (Richard, S. et al. (1990) J Biol Chem 265, 6098-6103), and lactoferrin (Teng, C. T. et al. (1992) Mol Endocrinol 6, 1969-1981) genes. They have variant EREs having sequences 5′-GGTCAcggTGGCC-3′ SEQ ID NO:6, 5′-GGTGAcctTGACC-3′ (SEQ ID NO:7) and 5′-GGTCAaggCGATC-3′ (SEQ ID NO:8), respectively; variations from the consensus are underlined. In certain embodiments the disclosed compositions, such as linked modulators attached to an AD can enhance transcription from non-consensus sites.

Glucocorticoid (GR), Progesterone (PR) and androgen (AR) receptors recognize hormone response elements (REs) having half-sites, 5′-AGAACA-3′, similar to that of the ERE (Freedman, L. P. (1992) Endocr Rev 13, 129-145; Mangelsdorf, D. J. et al. (1995) Cell 83, 835-839). Thyroid (TR), retinoid X (RXR), all-trans retinoic acid (RAR) and vitamin D (VD) receptors, on the other hand, interact with REs with half-sites, 5′-AGGTCA-3′, identical to that of ERE (Freedman, L. P. (1992) Endocr Rev 13, 129-145; Mangelsdorf, D. J. et al. (1995) Cell 83, 835-839). These sites however are arranged in direct (RXRE, RARE, VDRE, TRE) or inverted (G/P/A RE) repeats with various spacing (Freedman, L. P. (1992) Endocr Rev 13, 129-145; Mangelsdorf, D. J. et al. (1995) Cell 83, 835-839). ERE half-sites without spacing also produce a palindromic TRE (Freedman, L. P. (1992) Endocr Rev 13, 129-145; Mangelsdorf, D. J. et al. (1995) Cell 83, 835-839).

It is understood that the DNA binding sites to which the compositions bind can be from hormone response elements. Hormone response elements are well-known in the art and understood to be specific DNA sequences located in regulatory regions of many genes that allow for ligand-dependent regulation of gene expression through trans-acting factors. Such regions generally comprise two half-sites. Examples of response elements in the art include elements containing two consensus hexameric half-sites. The identity of a response element resides in three features: the sequence of the base pairs in the half-site, the number of base pairs between the half-sites and the relative orientation of the two half-sites. Thus each receptor protein dimer that binds the DNA has to recognize the sequence, spacing and orientation of the half-sites within their response element. The disclosed compositions can in certain embodiments bind any response element. For example, in certain embodiments the bind a hormone response element selected from the group of response elements consisting of Estrogen Response Element (ERE), Glucocorticoid Response Element (GRE), Androgen Response Element (ARE), Progesterone Response Element kYPD), TktiroWid Response Element (TRE), Retinoid X Response Element (RXRE), all-trans Retinoic Acid Response Elements (RARE), and Vitamin D Response Element (VDRE). As hormone response elements can comprise two half-sites, specifically disclosed are suppositions that bind a DNA binding site, wherein the DNA binding site comprises a first half-site and a second half-site.

Many half-sites of hormone response elements are well-known in the art. For example, one such half-site is the sequence 5′-AGAACA-3′ (SEQ ID NO. 1). Another example includes 5′-AGGTCA-3′ (SEQ ID NO: 2). Therefore, also disclosed are compositions that bind a DNA binding site wherein the DNA binding site comprises a first half-site and a second half-site, and wherein the first half-site comprises the sequence of 5′-AGAACA-3′ (SEQ ID NO: 1). Also disclosed are compositions, wherein the first half-site comprises the sequence of 5′-AGGTCA-3′ (SEQ ID NO: 2). Similarly disclosed are compositions that bind a DNA binding site wherein the DNA binding site comprises a first half-site and a second half-site, and wherein the second half-site comprises SEQ ID NO: 1. Also disclosed are compositions, wherein the second half-site comprises the sequence of SEQ ID NO: 2. It is understood and herein contemplated that half-sites can be repeats including direct and inverted repeats. Therefore in one embodiment the compositions interact with a first half-site and the second half-site in which the first and second half sites are direct repeats. In other embodiments the first and second half sites can in be inverted repeats. As the number of base-pairs between half-sites can be important for recognition by a DNA binding domain, specifically contemplated are compositions, wherein the first half-site and the second half site are not separated by a nucleotide. Also disclosed are compositions, wherein the first half-site and the second half site are separated by a 1, 2, 3, 4, 5, 10, or 15 nucleotide spacer.

Examples of DNA binding sites are well known in the art. Specifically disclosed is each and every DNA binding site. In particular, specifically disclosed are binding sites for hormone receptors. Thus, in one embodiment the compositions comprise a DNA binding domain bind that binds a DNA binding site from a hormone response element. It is understood that DNA binding sites can include but are not limited to 5′-AGAACAnnnTGTTCT-3′ (SEQ ID NO: 3), 5′-AGGTCAnAGGTCA-3′ (SEQ ID NO: 4), 5′-AGGTCATGACCT-3′ (SEQ ID NO: 5), 5′-GGTCAcggTGGCC-3′ (SEQ ID NO: 6), 5′-GGTGAcctGACC-3′ (SEQ ID NO: 7), 5′- GGTCAaggCGATC -3′ (SEQ ID NO: 8), or 5′-GGTCAnnnTGACC -3′ (SEQ ID NO: 9), GGGCAtcgTGACC (SEQ ID NO: 10) human angitensinogen gene ERE, GGTCGccaGGACC (SEQ ID NO: 11) human BCL-2 gene ERE, GGTCAggcTGGTC (SEQ ID NO: 12) human BRCA-1 gene ERE, GGCCGggcTGACC (SEQ ID NO: 13) human Calbinding-D9 gene ERE, GGCCGggcTGACC (SEQ ID NO: 14) human cathepsin D gene ERE, GGCCAcgaTGACA (SEQ ID NO: 15) human choline acetyltransferase.gene ERE, GGTGGcccTGACC (SEQ ID NO: 16) human complement3 gene ERE, GGTCAaggTGACC (SEQ ID NO: 17) human cytochorome c oxidase subunit Vila-related protein (COX7RP) gene ERE, GGTCAtggTGACC (SEQ ID NO: 18) human estrogen responsive finger protein gene ERE, GCAGGagcTGACC (SEQ ID NO: 19) human Progesterone receptor gene ERE, GGTCAgcgTGGCC (SEQ ID NO: 20) human pS2 gene ERE, AATCAgacTGACT (SEQ ID NO: 21) human VEGF gene ERE, GGTCAggcTGGTC (SEQ ID NO: 22) human Alu gene ERE.

9. Homology

It is understood that one way to define any known variants and derivatives or those that might arise, of the disclosed genes and proteins herein is through defining the variants and derivatives in terms of homology to specific known sequences. For example SEQ ID NO: 46 sets forth a particular sequence of an ERα and SEQ ID NO: 45 sets forth a particular sequence of the protein encoded by SEQ ID NO: 46, an ERα protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods EnzymoL 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

10. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 10 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 25 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

11. Nucleic Acids

There are a variety of molecules disclosed herein that are nucleic acid based, including for example the nucleic acids that encode, for example, ERα, ERβ, AR, PR, GR, TR, RXRα, RARα, and VPR as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an intemucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type stnucture when interacting with the appropriate target nucleic acid.

It is also possible to link other types of molecules (conjugates) to nucleotides or nucleotide analogs to enhance for example, cellular-uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

12. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modifed to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as (VP16)₂-CDC-(VP16)₂ into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a cornmonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities tanm hemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposornes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer 60:275-281, (1989); Bagshawe, et al., Br. J. Cancer. 58:700-703, (1988); Senter, et al., Bioconiugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research. 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In vivo/Ex vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoLiposomes. The transduced cells car then be infused (e.g., in a pharmaceutically acceptable carrier) or homotopically transplanted back into the subject per standard methods for the cell or tissue type. Standard methods are known for transplantation or infusion of various cells into a subject.

13. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Baneiji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

14. Peptides

a) Protein Variants

As discussed herein there are numerous variants of the activator protein and repressor protein that are known and herein contemplated. In addition, to the known functional strain variants there are derivatives of the elements that comprise the disclosed compositions which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well Known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 3
Amino Acid Abbreviations

| Amino Acid | Abbreviations |
|---|---|
| Alanine | Ala A |
| Allosoleucine | AIle |
| Arginine | Arg R |
| asparagines | Asn N |
| aspartic acid | Asp D |
| Cysteine | Cys C |
| glutamic acid | Glu E |
| Glutamine | Gln Q |
| Glycine | Gly G |
| Histidine | His H |
| Isolelucine | Ile I |
| Leucine | Leu L |
| Lysine | Lys K |
| phenylalanine | Phe F |
| Proline | Pro P |
| pyroglutamic acidp | pGlu |
| Serine | Ser S |
| Threonine | Thr T |
| Tyrosine | Tyr Y |
| Tryptophan | Trp W |
| Valine | Val V |

TABLE 4
Amino Acid Substitutions
Original Residue Exemplary Conservative Substitutions,
others are known in the art.

Ala; Ser
Arg; Lys; Gln
Asn; Gln; His
Asp; Glu
Cys; Ser
Gln; Asn, Lys
Glu; Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Substantial changes in function or immunological identity are made by selecting substitutions that are less conservative than those in Table 4, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are fequently post-translationally deamidated to the corresponding glutarnyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:46 sets forth a particular sequence of ERα and SEQ ID NO:45 sets forth a particular sequence of a ERα protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:45 is set forth in SEQ ID NO:46. In addition, for example, a disclosed conservative derivative of SEQ ID NO:24 is shown in SEQ ID NO: 81, where the isoleucine (I) at position 5 is changed to a valine (V). It is understood that for this mutation all of the nucleic acid sequences that encode this particular derivative of the ERα hinge domain are also disclosed.

It is understood that there are numerous amino acid and peptide analogs which can be incorporated into the disclosed compositions. For example, there are numerous D amino acids or amino acids which have a different functional substituent then the amino acids shown in Table 3 and Table 4. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Alnquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrurn of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

15. Pharmaceutical Carriers/Delivery of Pharamceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem. 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem. 4:3-9, (1993); Batteili, et al., Cancer Immunol. Immunother. 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters. such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be-present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

16. Compositions Identified by Screening with Disclosed Compositions/Combinatorial Chemistry

a) Combinatorial Chemistry

The disclosed compositions can be used as targets for any combinatorial technique to identify molecules or macromolecular molecules that interact with the disclosed compositions in a desired way.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, (VP16)₂-CDC-(VP16)₂, are also disclosed. Thus, the products produced using the combinatorial or screening approaches that involve the disclosed compositions, such as, (VP16)₂-CDC-(VP16)₂, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerfiul molecular genetic technique for identifying new regulatory molecules, specific to the protein of interest (Fields and Song, Nature 340:245-6 (1989)). Cohen et al., modified this technology so that interactions between synthetic or engineered peptide sequences could be identified which bind a molecule of choice. The benefit of this type of technology is that the selection is done in an intracellular environment.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

Screening molecules similar to (SID)₂-CDC-(SID)₂ for inhibition of hormone response element containing genes is a method of isolating desired compounds.

Molecules isolated which inhibit hormone response element containing genes can either be competitive inhibitors or non-competitive inhibitors.

17. Compositions with Similar Functions

It is understood that the compositions disclosed herein have certain functions, such as transcription modulation or binding DNA binding sites. Disclosed herein are certain structural requirements for performing the disclosed finctions, and it is understood that there are a variety of structures which can perform the same function which are related to the disclosed structures, and that these structures will ultimately achieve the same result, for example stimulation or inhibition of gene expression of a gene under control of a hormone response element.

D. Methods of using the compositions

1. Methods of Using the Compositions as Research Tools

The disclosed compositions can be used in a variety of ways as research tools. For example, the disclosed compositions, such as SEQ ID NOs:23-40 can be used to study the interactions between hormone receptors and gene expression, by for example acting as inhibitors of binding.

Specifically disclosed are methods of identifying a gene that is under transcriptional control of a hormone response element comprising contacting a cell containing the hormone response element with the compositions disclosed herein and monitoring the cell for changes in the transcription.

The compositions can be used for example as targets in combinatorial chemistry protocols or other screening protocols to isolate molecules that possess desired functional properties related to blocking hormone receptor induced gene expression.

The disclosed compositions can also be used diagnostic tools related to diseases such as cancer.

The disclosed compositions can be used to identify genes that are regulated by a particular receptor. For example, linked modulators that are designed from Estrogen receptor or the Progesterone receptor, can be made. Then various cell extracts, and systems can be used, to identify genes that are expressed in the presence of the constitutive activators, for example. The genes can be identified in any way desired, for example, by gel chromatography, sequencing, and differential display, or through the use of gene arrays, chips, or libraries. 2. Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

Thus specifically disclosed and contemplated herein are nucleic acids encoding the disclosed compositions. For example specifically disclosed are nucleic acids encoding compositions comprising the formula W-Z-X-Y, wherein W comprises a first DNA binding domain, X comprises a second DNA binding domain, Z comprises a linker, and Y comprises a transcription modulator, and wherein the composition binds a DNA binding site. Also disclosed are vectors comprising nucleic acids encoding the compositions disclosed herein.

One of the preferred characteristics of performing homologous recombination in mammalian cells is that the cells should be able to be cultured, because the desired recombination event occur at a low frequency. Therefore, also disclosed are cells comprising the disclosed vectors or nucleic acids.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal. It is therefore understood that the present invention discloses animals comprising cells which comprise nucleic acids encoding for the disclosed compositions.

3. Method of Treating Cancer

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastomalglioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

Therefore, specifically disclosed are methods of treating cancer in a subject comprising administering to the subject a composition comprising the formula W-X-Y, wherein W comprises a first DNA binding domain, X comprises a second DNA binding domain and Y comprises a transcription modulator, and wherein the composition binds a DNA binding site. Treatment can involve regulating uncontrolled cellular proliferation by acting directly on the proliferating cells or on indirectly by regulating factors inducing the proliferation. Thus also disclosed are methods of inhibiting the transcription of a gene comprising contacting a cell containing the gene with the composition the formula W-X-Y, wherein W comprises a first DNA binding domain, X comprises a second DNA binding domain and Y comprises a transcription modulator, wherein the composition binds a DNA binding site, and wherein the transcription modulator is a repressor domain. A further embodiment of the disclosed methods are methods, wherein the cell is in a subject. Also disclosed are methods, wherein the subject has cancer. The subject can be any animal, including a mammal, such as a mouse, rat, hamster, rabbit, ape, chimpanzee, orangutan, and a human.

As it is possible to regulate unregulated cellular proliferation through inhibition of hormone response element containing genes, it is also possible to regulate unregulated cellular proliferation through overexpression of hormone response element containing genes. Therefore, disclosed are methods of overexpressing a gene in a cell comprising contacting the gene with the composition the formula W-X-Y, wherein W comprises a first DNA binding domain, X comprises a second DNA binding domain and Y comprises a transcription modulator, wherein the composition binds a DNA binding site, and wherein the transcription modulator is an activation domain.

Thus a specific embodiment of the present invention are methods of treating cancer in a subject comprising administering to the subject the composition the formula W-X-Y, wherein W comprises a first DNA binding domain, X comprises a second DNA binding domain and Y comprises a transcription modulator, wherein the composition binds a DNA binding site, and wherein the composition causes the destruction of cancerous cells through the overexpression of genes under the control of hormone response elements.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

E. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W. H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:415.1 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chernical Ligation. Science, 266:776-779 (1994)). The first step is the chemoselective reaction of an unprotected synthetic peptide--thioester with another unprotected peptide segment containing an arnino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J.Biol.Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process Claims for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising each element of the disclosed compositions.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

Herein transactivators were engineered that specifically target ERE to regulate the expression of ERE-containing genes. The modular nature of ER permitted the design of a monomeric ERE binding module by co-joining two DNA binding domains with a hinge domain. Strong activation domains from other transcription factors were then integrated into this module to generate constitutively active ERE binding activators (EBAs). These activators potently induced the expression of only ERE-containing genes independent of ligand binding, dimerization, ER subtypes, promoter- and cell-context. Moreover, EBAs, as E2-ERα, differently altered the cell cycle progression in cells derived from breast cancer. While EBAs increased the cell population accumulated in the G1 phase of ER-negative MDA-MB-231 cells, they decreased the number of cells in the G1 phase in ER-positive MCF-7 cells. Thus, the targeted regulation of ERE-containing genes by the designer transregulators can provide an avenue to experimental biology and medicine.

a) Materials and Methods

(1) Construction of ERE Binding Proteins and Reporter Constructs.

The ERα, Flag-ERα expression and the reporter vectors were described previously (Muyan, M. et al. (2001) Mol Cell Endocrinol 182, 249-263; Yi, P. et al. (2002) Mol Endocrinol 16, 1810-1827; Yi, P. et al. (2002) Mol Endocrinol 16, 674-693). The expression vectors bearing cDNAs for PR and RXRα were provided by Drs. Edwin Milgrom and Ronald M. Evans, respectively (Misrahi, M. (1987) Biochem. Biophys. Res. Commun. 143 (McKenna, N. J. et al. (1999) Endocr Rev 20, 321-344), 740-748; Mangelsdorf, D. J. et al. (1990) Nature 345 (6272), 224-229) The promoter of the reporter vector is a TATA box or from the thymidine kinase (TK) that drives the expression of firefly luciferase cDNA as the reporter enzyme ((Muyan, M. et al. (2001) Mol Cell Endocrinol 182, 249-263; Yi, P. et al. (2002) Mol Endocrinol 16, 1810-1827; Yi, P. et al. (2002) Mol Endocrinol 16, 674-693). For the engineering of the reporter construct bearing one ERE derived from the human pS2, oxytocin, lactoferrin gene, or the consensus PRE or RXRE sequence, strands of oligomers (Integrated DNA Technologies, Inc., Coralville, Iowa), were annealed and inserted into the reporter vector linearized with appropriate enzymes. In all test sequence oligomers, the flanking sequences that surround a test sequence are identical, were described previously (Yi, P. et al. (2002) Mol Endocrinol 16, 674-693). For the engineering of ERE binding proteins, the cDNA for the C domain that encodes amino acids 181-263 or for the CD domain encoding residues 181-301 was generated by PCR using the ERα cDNA as template (Green, S. et al. (1986) Cold Spring Harbor Symposia on Quantitative Biology 51, 751-758). The 5′ and 3′ ends of cDNAs for both constructs contain multiple restriction enzyme sites for subsequent insertions of cDNAs for the activation domain (AD) of the herpes simplex virion protein, VP16 and/or the p65 subunit of the nuclear factor κB, NFκB. The constructs were separately sub-cloned into the modified pBluescript-II KS (+) (Stratagene, La Jolla, Calif.) that bears sequences encoding for a 5′ Flag epitope and a 3′ 6×His tag. For the generation of fusion CC, CCD or CDC, the appropriate fragments were joined for a corresponding cDNA. In the construction of the CDNA for the AD of VP16, pVP16 of the mammalian two-hybrid system (Invitrogen, Carlsbad, Calif.) was used as the template. The AD of VP16 contains amino-acid residues 403-490. The cDNA for the AD of p65 encompassing residues 416-550 (Moore, P. A. et al. (1993) Mol Cell Biol 13, 1666-1674) was generated by PCR using a human testis cDNA library (Clontech Laboratories, Palo Alto, Calif.). The CDC and ERα mutants deficient in DNA binding contain amino acid substitutions that replace two of the zinc-coordinating Cys of the first zinc-finger of the C domain at position 202 and 205 (Kumar, V. et al. (1988) Cell 55, 145-156) with His. These were constructed by overlapping PCR. In the generation of the mutant VP16 with reduced transactivation potential (Regier, J. et al. (1993) Proc Natl Acad Sci USA 90, 883-887), Phe at position 442 was replaced with Pro by overlapping PCR. All constructs were sequenced. The generation of the pS2 and C3 promoter was described previously (Yi, P. et al. (2002) Mol Endocrinol 16, 1810-1827). The 337-bp fragment (−334 to +3, 1 as the ATG codon) oxytocin promoter construct was generated from the genomic DNA of MCF-7 cells using primers based on the published sequence (Richard, S. et al. (1990) J Biol Chem 265, 6098-6103). In the engineering of reporter plasmid bearing Col promoter, a 145-bp fragment (−142 to +3, +1 as the start codon) was obtained by PCR from the genomic DNA of MCF-7 cells using primers based on the published sequence (Angel, P. et al. (1987) Mol Cell Biol 7, 2256-2266). Similarly, a 527-bp PCR fragment (−491 to +36, +1 indicates the transcription initiation site) of the RARα gene (Brand, N. J.et al. (1990) Nucleic Acids Res 18, 6799-6806) was generated. PCR fragments were inserted into pGL3-Basic that contains the firefly luciferase enzyme cDNA as the reporter enzyme. For the cell cycle analysis, the cDNA for the enhanced green fluorescent protein driven by the CMV promoter was excised from the pEGFP-C1 vector (Invitrogen, Carlsbad, Calif.) was inserted into the backbone of pCDNA 3.1 (−) (Invitrogen) with appropriate enzyme. The cDNA for ERα, CDC or an EBA was then inserted into the multiple cloning site of the resultant vector.

(2) Synthesis and Detection of ERE Binding Proteins in vitro.

Transcription/translation of constructs in vitro was performed with a rabbit reticulocyte system (Promega, Madison, Wis.) as described previously (Muyan, M. et al. (2001) Mol Cell Endocrinol 182, 249-263). The reaction mixture, which contained an amino acid mixture that lacked Met, was divided into two aliquots. While one aliquot received 1 μl of [³⁵S]-Met (>1000 Ci/mmol; at 10 mCi/ml; NEN Life Sciences, Boston Mass.), the other received 1 μl of 1 mM unlabeled Met to a total of 25 μl reaction. Equal aliquots (5 μl) containing radiolabeled proteins were then subjected to electrophoresis on 4-18% gradient SDS-PAGE under reducing conditions. The gel was immersed in 1 M sodium salicylate for 30 min, dried and visualized by a PhosphoImager (Molecular Dynamics, Sunnyvale, Calif.) or by an X-OMAT film (Kodak, Rochester, N.Y.). The corresponding unlabeled aliquots were used in EMSA. For the estimation of relative DNA binding ability of the CDC protein we also used partially purified CDC from a bacterial expression system that utilizes pGEX-2TK as the expression vector (Amersham-Pharmacia Biotech, Piscataway, N.J.). The GST-CDC fusion protein was expressed in E. coli BL21 (DE3)Lys cells and purified using GST Purification Modules (Amersham-Pharmacia) following in-column thrombin digestion of the fusion protein. Protein concentrations in eluates were estimated using a Bio-Rad Protein Assay Kit (Bio-Rad Labs, Hercules, Calif.). Equal aliquots of GST-fusion proteins were resolved by SDS-PAGE and visualized by Coomassie staining of the gel or a horse-radish peroxide conjugated 6×His antibody (Amersham) by Western blotting. Human recombinant ERα expressed in baculovirus-infected insect cells was purchased from Panvera, Inc. (Madison, Wis.).

(3) Electrophoretic Gel Mobility Shift Assay (EMSA).

Double stranded oligomers were annealed and [³²P] end-labeled with a 3,000 Ci/mmol specific activity isotope and used in EMSA assays as described (Muyan, M. et al. (2001) Mol Cell Endocrinol 182, 249-263; Yi, P. et al. (2002) Mol Endocrinol 16, 674-693). One fmol end-labeled DNA was incubated with an equal amount of reaction mixture (5 μl) in a binding buffer in a total volume of 20 μl for 1 h. Reactions were resolved by 8% native PAGE. The dried gel was visualized and quantified by Phospholmager (Molecular Dynamics). The affinity of CDC in comparison with ERα for the consensus ERE was determined by EMSA competition assay. Equal concentrations of functional proteins determined by the same amount of bound (shifted) radiolabeled ERE in the preceding EMSA were incubated with 0.05 nM of [³²P] end-labeled consensus ERE for 30 min. This was followed by incubation with 0 to 8 nM unlabeled consensus ERE for 30 min. Reactions were then resolved by 8% native PAGE. The concentration of unlabeled ERE required to reduce ER complex formation with the labeled ERE by 50% (IC₅₀) is proportional to the affinity of ER for the unlabeled ERE (Yi, P. et al. (2002) Mol Endocrinol 16, 674-693).

(4) Missing Nucleoside Hydroxyl Radical Assay (HRA).

HRA was performed as described (Yi, P. et al. (2002) Mol Endocrinol 16, 674-693). In brief, the bottom strand of the double stranded DNA containing ERE was 3′ end-labeled and randomly cleaved by hydroxyl radical. DNA was then incubated with proteins on ice for 30 min. Bound and free EREs were excised from the gel, eluted, and equal counts were subjected to 18% sequencing gel electrophoresis. Maxam-Gilbert G-specific sequencing reactions were performed simultaneously.

(5) Cell Culture and Transfection.

Culturing and transfection of the mammalian COS-1 and CHO MDA-MB-231 cells were described previously (Muyan, M. et al. (2001) Mol Cell Endocrinol 182,249-263; Yi, P. et al. (2002) Mol Endocrinol 16, 1810-1827), with the exceptions that 48 well cell culture plates were used and Trans-IT LTI transfection reagent (Mirus Corp., Madison, Wis.). Luciferase assays were performed with a Dual Luciferase Assay kit (Promega) according to the manufacture's recornmendations. Steroid hormones and 9-cis retinoic acid were purchased from Sigma-Aldrich (St. Louis, Mo.). For transfection of MDA-MB-231 and MCF-7 cells, the cells were incubated for three days in a phenol red-free DMEM supplemented with 10% charcoal-dextran treated fetal bovine serum (CD-FBS). Cells were transfected with appropriate expression vectors using Lipofectamine 2000 transfection reagent (Invitrogen).

(6) Cell Cycle Analysis.

MDA-MB-231 and MCF-7 cells were grown as described above. Cells were then transiently transfected with the EGFP vector bearing a cDNA for ERα, CDC or an EBA. Twenty-four hours after transfection, cells were collected by trypsinization, washed in phosphate buffered saline (PBS) and fixed with 1% paraformaldehyde in PBS for one hour. Cells were permeabilized in 70% Ethanol overnight and stained with 50 μg/ml propidium iodide (PI, Sigma-Aldrich) for one hour for flow cytometry analysis. Samples were analyzed on a FACS EPICS Elite EPS flow cytometer (Coulter Corp., Hialeah, Fla.). The percentages of cells at G1 phase were assessed among treatment groups and expressed as percent change compared to cells transfected with EGFP vector bearing no cDNA.

b) Results

(1) Design of an ERE Binding Module

ERE binding proteins were engineered as potent activators of ERE-containing genes independent of ligand, ER subtypes, dimerization, promoter- and cell-type. Proteins with various configurations were initially generated to obtain an ERE binding module that interacts efficiently with the consensus ERE. ER recognizes an ERE as a dimer with interaction primarily mediated by a dimerization interface located in the ligand binding domain and through a weak interaction surface in the C domain (Parker, M. G. (1998) Biochem Soc Symp 63, 45-50; Hall, J. M. et al. (2001) J Biol Chem 276, 36869-36872). Each C domain of the ER dimer makes analogous contacts with one of the inverted motifs, resulting in a rotationally symmetric structure (Glass, C. K. (1994) Endocr Rev 15, 391-407; Luisi, B. F.et al. (1994) Vitam Horm 49, 1-47). Although the isolated C domain can interact with a half-site of the consensus ERE as a monomer, albeit with a substantially lower DNA binding affinity than that of the parent receptor, it requires ERE-dependent dimerization for the formation of a stable C-ERE complex (Schwabe, J. W. et al. (1993) Cell 75, 567-578; Nardulli, A. M. et al. (1991 J Biol Chem 266, 24070-24076; Kuntz, M. A. et al. (1997) J Biol Chem 272, 27949-279456). Consistent with this, a previous study indicated that dimerization of the C domains through an artificial linker enhances binding to ERE sequences (Kuntz, M. A. et al. (1997) J Biol Chem 272, 27949-279456). ERs share 97% amino-acid identity in their DBDs (Mosselman, S.et al. (1996) FEBS Letters 392, 49-53). This is reflected in their abilities to bind various ERE sequences with similar specificity and affinity by interacting with the same nucleosides (Yi, P. et al. (2002) Mol Endocrinol 16, 674-693). Thus, the C domain of ERα was chosen as the template protein in the engineering of ER subtype-independent ERE binders. The engineered proteins consisted of two C domains of ERα co-joined without (CC and CCD) or with the D domain as a linker (CDC) by the genetic conjugation approach (FIG. 1A) (Muyan, M. et al. (2001) Mol Cell Endocrinol 182, 249-263). The choice of the D domain as a linker was based on the following observations. First, although the isolated C domain is the minimal region for ERE binding, the immediate amino-terminal residues of the D domain contribute significantly to the DNA binding affinity by creating a stable interaction between the half-site-bound monomers (Green, S. et al. (1986) Cold Spring Harbor Symposia on Quantitative Biology 51, 751-758; Schwabe, J. W. et al. (1993) Cell 75, 567-578; Kumar, V. et al. (1987) Cell 51, 941-951; Mader, S. et al. (1993) Nucleic Acids Res 21, 1125-1132). Second, the D domain, providing flexibility between the amino- and carboxyl-termini of the receptor, also contains patches of amino-acids that act cooperatively for the nuclear localization of ERα (Picard, D. et al. (1990) Cell Regulation 1, 291-299).

Analyses by fluorography of ERE binding proteins produced in a transcription/translation system in vitro indicated that the fusion cDNAs encode proteins with expected Mᵣs and are synthesized at similar molar concentrations (FIG. 1B). Only CDC comprised of two C domains co-joined by the D domain bound to the consensus ERE sequence in EMSA, unless an antibody-directed to the amino-terminal Flag (FIG. 1C) or the carboxyl-terminus 6×His epitope was added into the reactions. This indicates that the bivalency of the antibody tethers.two C-domains in a dimer configuration that allows the protein to bind to ERE (Kuntz, M. A. et al. (1997) J Biol Chem 272, 27949-279456). Thus, the covalent conjugation of two C domains brought to close proximity by the D domain was the optimal approach to allow CDC to bind efficiently to the consensus ERE.

(2) Biochemical Properties of the ERE Binding Module.

CDC, as ERα, did not interact with the DNA fragment bearing an ERE half site, 5′-GGTCA-3′ (FIG. 1D) (Muyan, M. et al. (2001) Mol Cell Endocrinol 182, 249-263; Klinge, C. M. et al. (1997) Nucleic Acids Res 25, 1903-1912). Substitutions of two of zinc-coordinating Cys with His residues of the first zinc-finger in the C domain of ERα prevent the receptor from interacting with DNA (Kumar, V. et al. (1988) Cell 55, 145-156). CDC mutants with the equivalent mutation in the first (C*DC), second (CDC*) or both (C*DC*) C domains of the protein were also generated to address whether both DNA binding domains are required for CDC to bind to ERE. Although synthesized at levels comparable to those of the parent proteins as assessed by fluorography (FIG. 1E), the DNA binding defective mutant C*DC, as other CDC mutants, and the mutant ERα*, showed no ERE binding irrespective of the addition of the flag antibody (FIG. 1F).

Moreover, the recombinant CDC and ERα occupied both half-sites of the consensus ERE by contacting with the same nucleosides (FIG. 1G). This was revealed by the missing nucleoside hydroxyl radical assay (HRA) which allows the analysis of DNA-protein interaction at a single-nucleotide resolution (Yi, P. et al. (2002) Mol Endocrinol 16, 674-693; Hayes, J. J. et al. (1989) Biochemistry 28, 9521-9527). HRA is based on the expectation that if a base important for binding were missing in a particular DNA molecule, the protein binding affinity would be adversely affected. Both CDC and ERα interacted with the same nucleosides of the consensus ERE with similar strengths. Thus, the monomer CDC binds to ERE in a manner similar to the dimer ERα.

The competition assay revealed that recombinant CDC bound to ERE with an apparent K_{d} of 1.49±0.16 nM as estimated byEMSA (FIG. 1H). Although the DNA binding affinity of CDC is six-fold lower than that of ERα (K_{d} of 0.26±0.03 nM), CDC effectively competed with ERα for binding to ERE (FIG. 1I). These results collectively indicate that CDC can serve as an effective ERE binding module for the engineering of ERE-binding transactivators.

(3) Engineering and Functional Analysis of ERE Binding Activators.

To generate potent regulators of ERE-containing genes, an ERE binding protein with activation function, designated as the ERE binding activator (EBA), was produced. The strong activation domain (AD) of the herpes simplex virion protein, VP 16 (Triezenberg, S. J. et al. (1988) Genes Dev 2, 718-729; Sadowski, I. et al. (1988) Nature 335, 563-564), or the p65 subunit of the nuclear factor κB, NFκB, (Moore, P. A. et al. (1993) Mol Cell Biol 13, 1666-1674) as a single copy was genetically fused to the amino-(VP16-CDC or p65-CDC) or carboxyl-terminus (CDC-VP16 or CDC-p65) of CDC. The effectiveness of these EBAs to activate gene transcription in ER-negative African green monkey kidney epithelial (COS-1) cells was tested. Cells were transiently transfected with an expression vector without or with a cDNA for ERα or for an EBA, together with a reporter plasmid bearing none, one or two consensus ERE sequences, placed at the upstream of a simple TATA box promoter that drives thefirefly luciferase cDNA. Also co-transfected were cells with a reporter plasmid bearing a cytomegalovirus (CMV) promoter driving the expression of renilla luciferase cDNA for monitoring transfection efficiency. Normalized activity from each reporter was compared to the basal activity from the reporter construct bearing one ERE in response to the parent expression vector (pM²) in the absence of ligand, which was set to one.

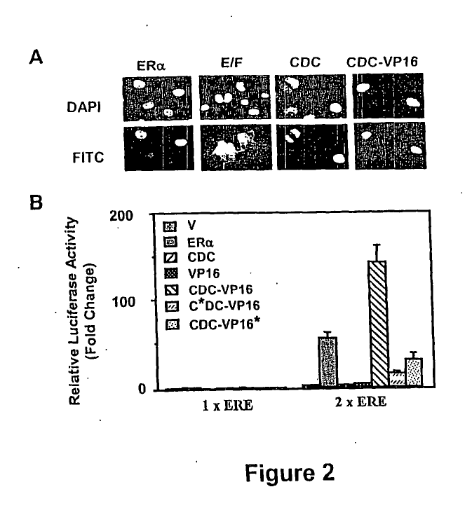

CDC-VP16 (or p65-CDC), just as ERα, localized to the nucleus in the absence (FIG. 2A) or presence of an ER-ligand, while the parent vector produced no intracellular staining. The nuclear staining is protein-type dependent, because the truncated ERα variant (EF) that lacks the A/B region along with the C and D domains showed both cytoplasmic and nuclear staining (Yi, P. et al. (2002) Mol Endocrinol 16, 674-693).

Expression of EBAs had no effect on gene transcription from the TATA box promoter bearing no ERE. ERα increased luciferase activity two-fold in response to a physiological concentration, 10⁻⁹ M, of E2 from one ERE (1×ERE) (FIG. 2B, see also Table 1). Similarly, CDC-VP16 (or p65-CDC, and see below) induced the reporter enzyme activity about four-fold in the absence or presence of E2. However, both E2-ERα and CDC-VP16 (shown in the absence of E2) augmented enzyme activity synergistically from two EREs (2×ERE).

TABLE 1
Effects of increasing number of ADs and
of EREs on EBA-driven transactivations

| Construct | 1× | 2× |
|---|---|---|
| Vecto | 1.0 ± 0.0 | 1.1 ± 0.1 |
| ERα | 2.0 ± 0.3 | 28. ± 2.4 |
| VP16- | 1.8 ± 0.3 | 8.8 ± 0.5 |
| CDC- | 3.7 ± 0.5 | 57. ± 5.0 |
| VP16-CDC- | 13. ± 2.0 | 17 ± 20. |
| (VP16)₂- | 9.3 ± 1.5 | 11 ± 7.7 |
| CDC- | 27. ± 4.2 | 23 ± 24. |
| p65- | 2.4 ± 0.4 | 43. ± 5.2 |
| CDC- | 3.9 ± 0.8 | 79. ± 5.5 |
| p65-CDC- | 34. ± 3.9 | 67 ± 10 |
| (p65)₂-CDC | 45. ± 6.2 | 51 ± 50. |
| CDC-(p65)₂ | 29. ± 5.0 | 39 ± 33. |

If the total effect of ER or the activator on responses from tandem ERE sequences was greater than the sum of the effects observed with each element independently, the transcription was considered synergistic. Moreover, the magnitude of transactivation induced by the EBA from the tandem ERE construct was two-fold greater than that observed with the E2-ERα complex. CDC or VP16 alone did not alter the transcription. Similarly, the ERE binding deficient mutant C*DC-VP16 or the CDC-VP16* variant with an impaired activation function due to a point mutation in the AD of VP16 (Cress, W. D. et al. (1991) Science 251, 87-90) had a dramatically reduced activation potency. Collectively, these results indicate that the ability of the EBA to induce gene transcription from an ERE is dependent on the structural integrity of the DNA binding domains of CDC and on the presence of an AD. Thus, the CDC with an AD can furnish the necessary scaffold for the engineering of potent EBAs.

(4) Effects of Increasing the Number of a Single AD.

Since an EBA with one copy of an AD augmented reporter activity synergistically from tandem ERE sequences (ERE-dependent synergy), it was asked whether an EBA with two copies of an AD induces transactivation from one ERE at levels observed from two EREs. An EBA with a single copy of an AD fused to both termini, or with two copies of an AD placed at the amino or carboxyl-terminus was generated and tested its ability to induce gene transcription from one ERE in transfected COS-1 cells. Activators with one AD of VP16 or of p65 at each terminus (VP16-CDC-VP16 or p65-CDC-p65) or two copies at either terminus [(VP16)₂-CDC or CDC-(VP16)₂, (p65)₂-CDC or CDC-(p65)₂] enhanced the reporter activity dramatically compared to those with a single AD (Table 1). These results indicate that in addition to the synergy observed with the tandem ERE sequences, the presence of two ADs fused as single copies to both domains or as tandem copies to either domain can also synergize from one ERE (domain-dependent synergy). This is reminiscent of the synergistic activation observed between the AF-1 and AF-2 domains of ERα which show minimal activation potential when they are expressed alone (Yi, P. et al. (2002) Mol Endocrinol 16, 1810-1827; Benecke, A. et al. (2000) EMBO Reports 1, 151-157; Kraus, W. L. et al. (1995) Proc Natl Acad Sci USA 92, 12314-12318). Interestingly, CDC-(VP16)₂ had higher transcription potency than that observed with VP16-CDC-VP16 or (VP16)₂-CDC. This result indicates that the AD of VP16 displays a position dependency in inducing transcription, being more effective when it is placed at the carboxyl-terminus of CDC. This is similar to observations with the zinc-finger-based activators with a single AD of VP-16 (Zhang, L. et al. (2000) J Biol Chem 275, 33850-33860; Liu, P. et al.(2001) J Biol Chem 276, 11323-11334). This was also the case for EBAs with the AD of p65. While CDC-(p65)₂ and p65-CDC-p65 showed similar potencies, (p65)₂-CDC was the most potent activator. Remarkably, EBAs with two ADs positioned at either terminus or both termini also retained their abilities to induce transcription synergistically from tandem EREs (Table 1).

Thus, an increase in the number of ADs further enhances the biopotency of an EBA.

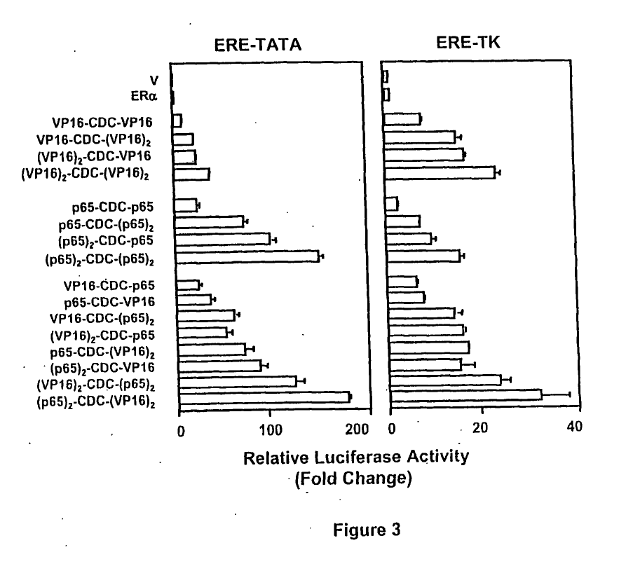

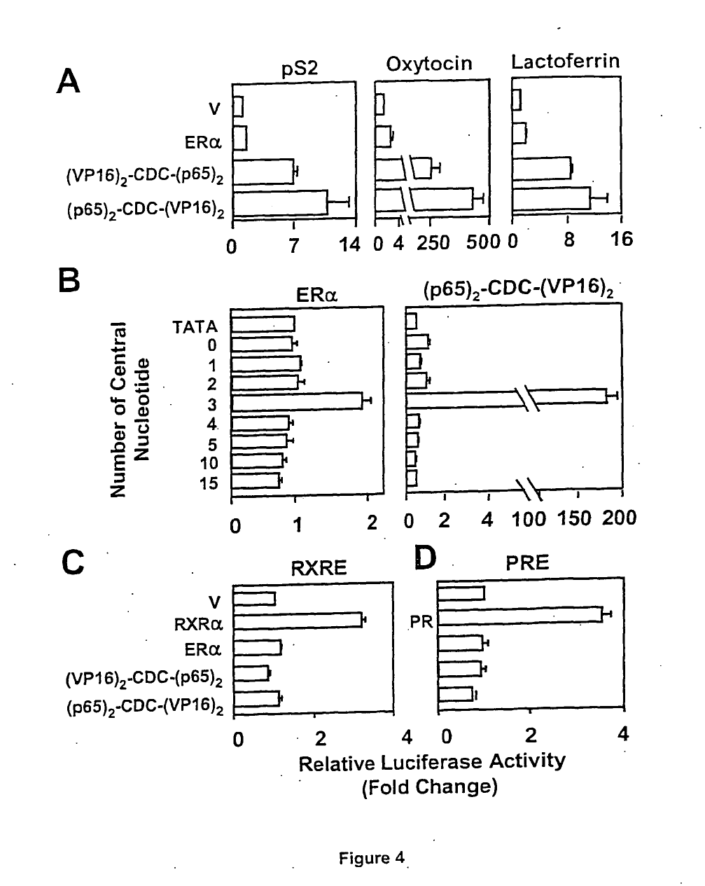

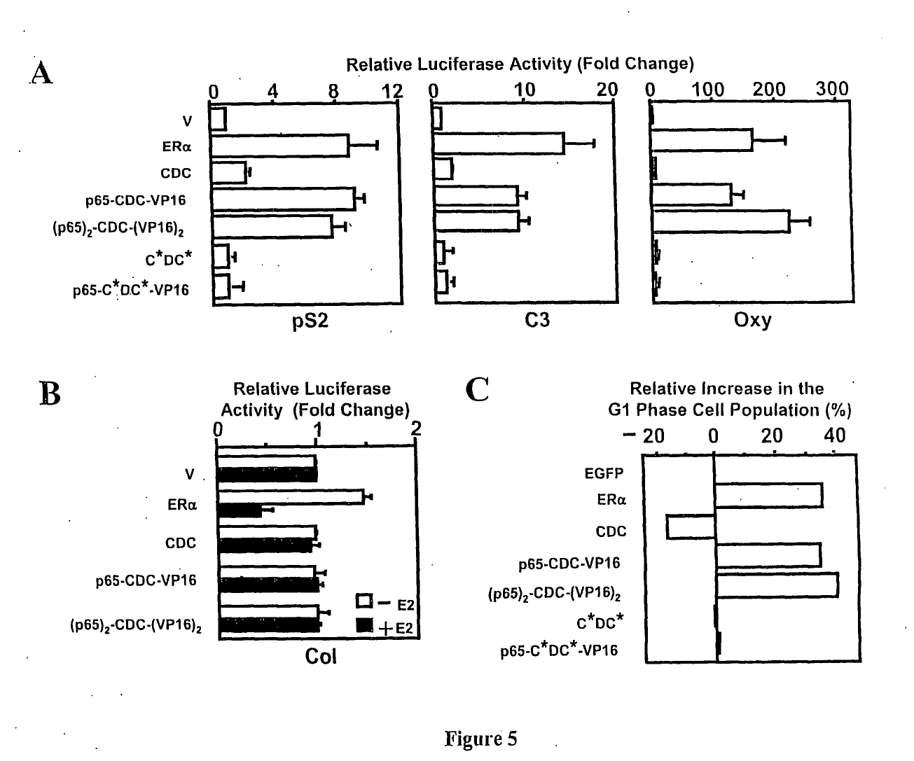

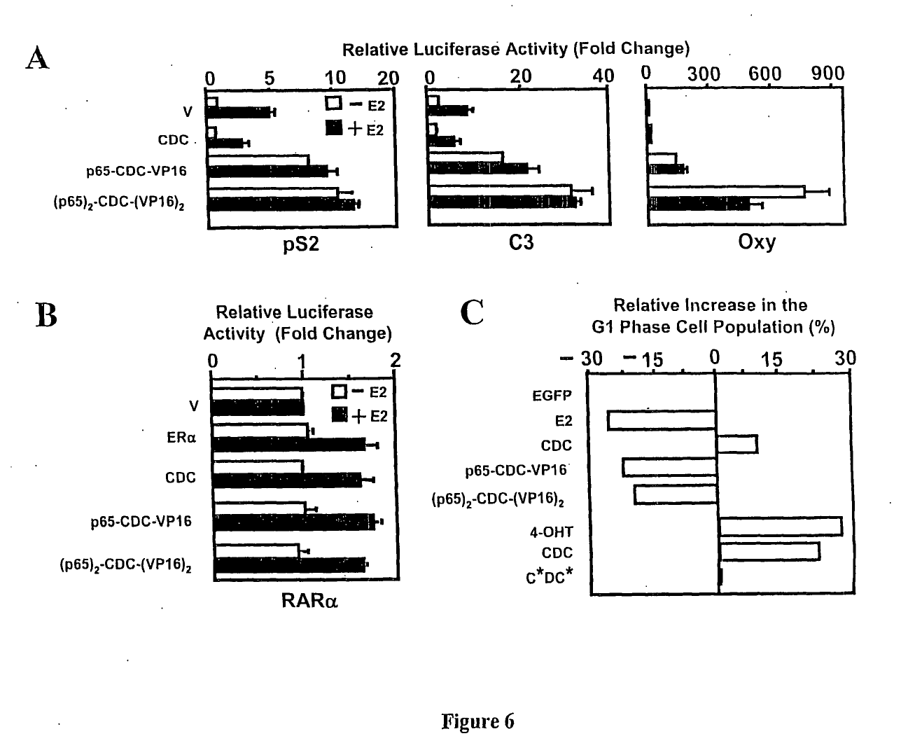

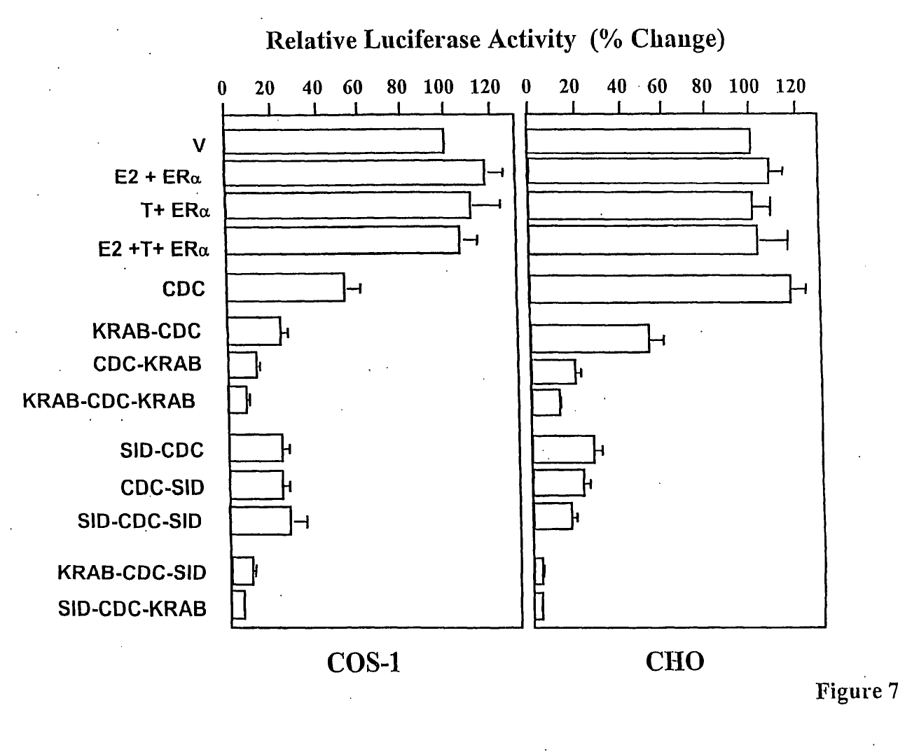

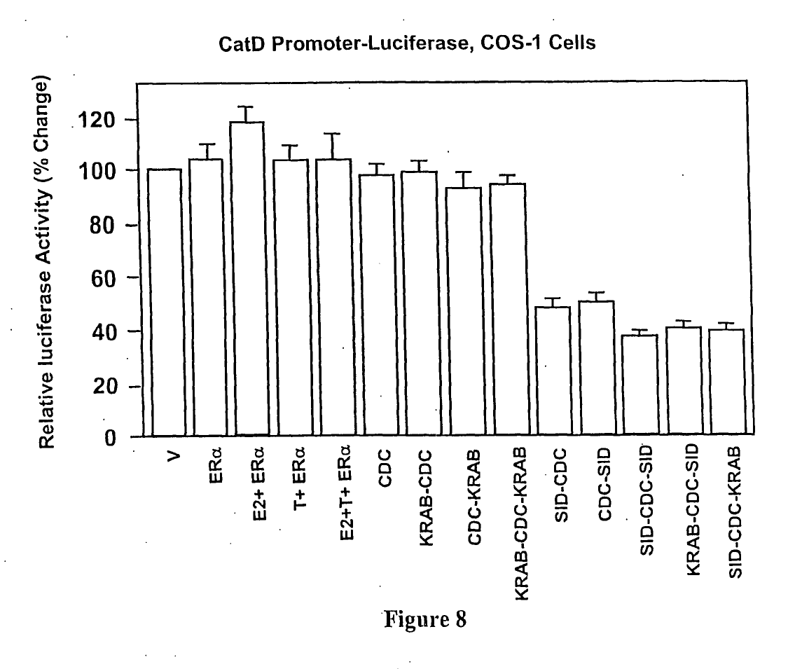

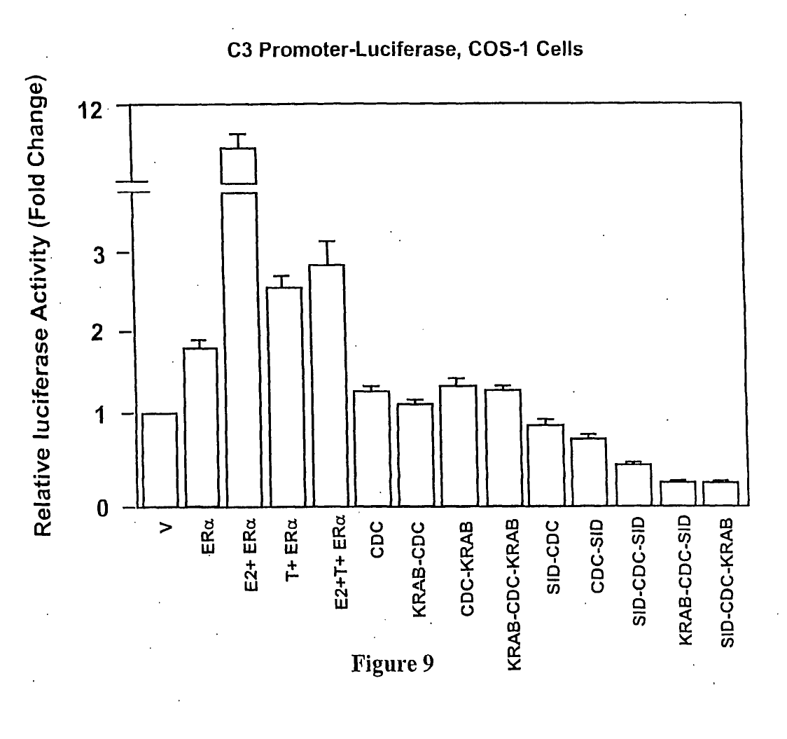

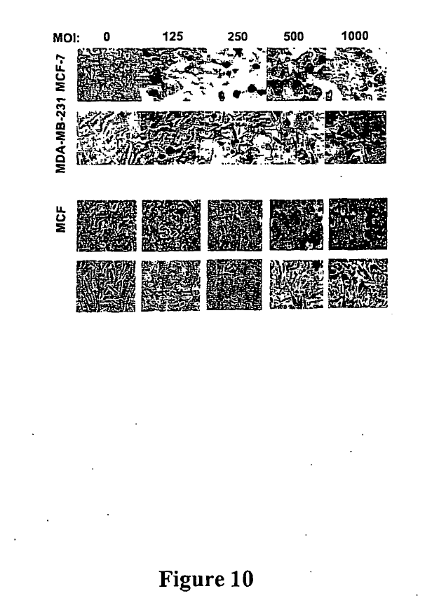

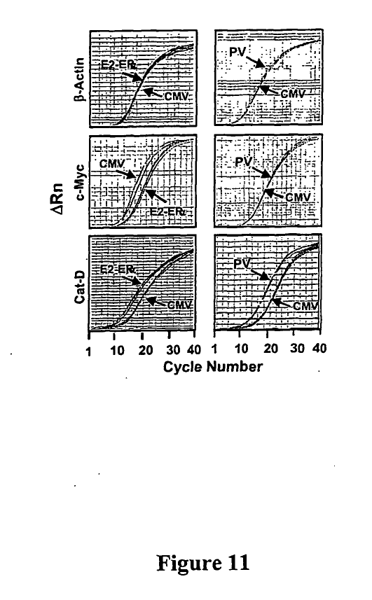

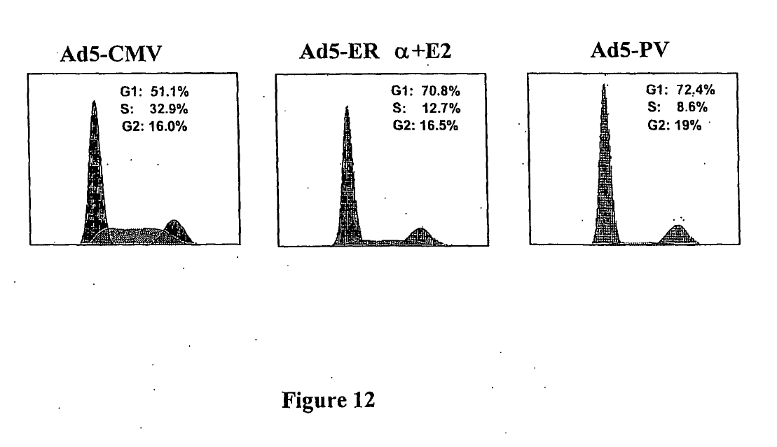

Increases in the copy number of an AD further augmented the biopotency of the activator. An EBA bearing three [VP16-CDC-(VP16)₂ or (VP16)2-CDC-VP16] or four copies [(VP16)₂-CDC-(VP 16)₂] of VP16-AD synergistically increased the reporter enzyme activity compared to EBAs with two copies of ADs of VP16 positioned at either or both termini (FIG. 3). Similar results were observed with the AD of p65 conjugated EBAs (FIG. 3). Thus, increasing the number of an AD provides the EBA with more transcriptional strength.

(5) Effects of Promoter-Type on the Biopotency of an EBA.

It is also evident that EBAs with p65-AD are more potent activators compared to those bearing the VP16-AD when tested with an ERE-driven TATA box promoter (FIG. 3). Since the promoter context can affect the ability of a transactivator to induce gene transcription, also tested was whether the type of an AD would differentially alter the ability of EBAs to induce transcription from the TK promoter without or with a juxtaposed consensus ERE (ERE-TK). EBAs had no effect on transcription from TK promoter bearing no ERE. EBAs, on the other hand, potently induced transcription with increasing magnitude as a consequence of an increase in the number of ADs. Interestingly, the AD of VP-16, in contrast to findings observed with the TATA box promoter, was more potent than that of p65 (FIG. 3) when tested with TK promoter-driven reporter construct. These results indicate that the type of a promoter can affect the transcriptional strength of an AD species.

The recruitment of some components of the basal transcription machinery that includes TFIIB, TFIID and DRIP/ARC complex by the AD of VP-16 appears to be the initial event for establishing a functional transcriptome (Stringer, K. F. et al. (1990) Nature 345, 783-786; Lin, L. et al. (1991) Biochimica et Biophysica Acta 1082, 177-184; Rachez, C. et al. (1999) Nature 398, 824-828). Whereas, the ability of the AD of p65 to stably interact with the p160 family of co-factors and with p300/CBP provides the complex with the potent transactivation potential (Gerritsen, M. E. et al. (1997) Proc Natl Acad Sci USA 94, 2927-2932; Uesugi, M. et al. (1997) Science 277, 1310-1313). It was reasoned that an EBA with ADs of VP16 and p65 at either terminus can circumvent the limitations on the biopotency of an EBA imposed by the promoter-type through a functional integration of diverse co-factors/regulators in comparison to those recruited by a single AD species. Indeed, VP16-CDC-p65 or p65-CDC-VP16 activated transcription from both the TATA box and the TK promoter constructs bearing one ERE more potently than VP16-CDC-VP16 (FIG. 3). p65-CDC-VP16, on the other hand, displayed more transcriptional strength compared to p65-CDC-p65 or VP16-CDC-p65.

Since an increase in diversity of ADs further augments the transcriptional potency of an activator, whether EBAs bearing ADs of both VP16 and p65 with increasing numbers can generate more potent transactivators than those with a single copy of each AD species was investigated. Genetic conjugation of VP-16 and/or of p65 to the amino- and/or carboxyl-terminus of VP16-CDC-p65 or of p65-CDC-VP16 led to a further enhancement in the transcriptional potencies of EBAs as a result of an increase in the number of ADs. (VP16)₂-CDC-(p65)₂ and (p65)₂-CDC-(VP16)₂ were the most potent activators, having capabilities of enhancing the reporter enzyme activity from either one (FIG. 3) or two ERE-driven reporter construct (Table 2) more than 100-fold compared to E2-ERα. Similarly, (VP16)₂-CDC-(p65)₂ and (p65)₂-CDC-(VP16)₂ were also more potent activators than those with a single AD species for inducing transcription from the TK promoter construct. Thus, an increase in diversity of ADs of EBA circumvents the promoter-context dependent limitations on the transcription strength of an AD.

TABLE 2
Effects of the cell-type on transactivation abilities of EBAs

| | CHO | | MDA-MB-231 | |
|---|---|---|---|---|
| | 1× | 2× | 1× | 2× |
| Construct | ERE | ERE | ERE | ERE |
| Vector | 1.00 ± 0.00 | 1.23 ± 0.12 | 1.00 ± 0.00 | 1.03 ± 0.08 |
| ER | 1.76 ± 0.14 | 10.1 ± 0.50 | 1.76 ± 0.12 | 39.7 ± 10.2 |
| (VP16)₂- | 41.6 ± 1.82 | 162 ± 9.18 | 11.8 ± 3.89 | 367 ± 100 |
| CDC- | | | | |
| (p65)₂- | 44.5 ± 5.41 | 163 ± 11.8 | 21.9 ± 3.71 | 349 ± 77.6 |
| CDC- | | | | |

(6) Effects of the Cell-Type on EBA-Induced Transactivation.

The type and availability of a myriad of activators, co-factors/integrators and components of the general transcriptional machinery determine the cell-specific transcriptional responses. To examine whether EBAs induce transcription more potently than E2-ERα independent of the cell-type, EBAs were transfected into ER-negative Chinese Hamster Ovary (CHO) and human breast adenocarcinoma (MDA-MB-231) cells (Table 2). EBAs induced the reporter enzyme activity potently in all transfected cells. Although the magnitude of transcription from one or two EREs varied, EBAs induced transcription more potently compared to E2-ERα in all cells.

(7) Transcriptional Responses of Non-Consensus EREs to EBAs.

The human pS2 (Nunez, A. M. et al. (1989) EMBO J 8, 823-829), oxytocin (Richard, S. et al. (1990) J Biol Chem 265, 6098-6103), and lactoferrin (Teng, C. T. et al. (1992) Mol Endocrinol 6, 1969-1981) genes confer E2-ERα responsiveness through variant ERE sequences 5′-GGTCAcggTGGCC-3′, 5′-GGTGAcctTGACC-3′ and 5′-GGTCAaggCGATC-3′, respectively; variations from the consensus are underlined. To assess whether EBAs induce transcription potently from one non-consensus ERE sequence placed upstream of the TATA box promoter, COS-1 cells were co-transfected with (VP16)₂-CDC-(p65)₂ or (p65)₂-CDC-(VP16)₂ (FIG. 4A). Both activators strongly augmented the reporter enzyme activity compared to E2-ERα. Thus, EBAs also induce gene transcription from non-consensus ERE-driven reporter constructs.

(8) The Functional Conservation of Sequence Recognition by EBAs.

Glucocorticoid (GR), Progesterone (PR) and androgen (AR) receptors recognize hormone response elements (REs) having half-sites, 5′-AGAACA-3′, similar to that of the ERE (Freedman, L. P. (1992) Endocr Rev 13, 129-145; Mangelsdorf, D. J. et al. (1995) Cell 83, 835-839). Thyroid (TR), retinoid X (RXR), all-trans retinoic acid (RAR) and vitamin D (VD) receptors, on the other hand, interact with REs with half-sites, 5′-AGGTCA-3′, identical to that of ERE (Freedman, L. P. (1992) Endocr Rev 13, 129-145; Mangelsdorf, D. J. et al. (1995) Cell 83, 835-839). These sites however are arranged in direct (RXRE, RARE, VDRE, TRE) or inverted (G/P/A RE) repeats with various spacing (Freedman, L. P. (1992) Endocr Rev 13, 129-145; Mangelsdorf, D. J. et al. (1995) Cell 83, 835-839). ERE half-sites without spacing also produce a palindromic TRE (Freedman, L. P. (1992) Endocr Rev 13, 129-145; Mangelsdorf, D. J. et al. (1995) Cell 83, 835-839). The DNA binding regions of these hormone receptors contains two zinc finger-like modules that fold to form a single functional domain. The protein dimer contacts nucleotides in adjacent major grooves on one face of the DNA helix containing a response element. Distinct residues in a region of the first zinc-finger module of the C domain, P-box, determine the DNA binding specificity (sequence discrimination) through a protein-DNA interaction. Whereas residues in the second zinc-finger-like module, the D-box, are involved in the discrimination of half-site spacing through a protein-protein interaction (dimerization) that influence the spacing and relative orientation of the two DNA associated monomers (Freedman, L. P. (1992) Endocr Rev 13, 129-145; Mangelsdorf, D. J. et al. (1995) Cell 83, 835-839). These structural constraints prevent each hormone receptor from binding promiscuously to REs by the recognition of the central spacing, sequence and arrangement of half-sites (Freedman, L. P. (1992) Endocr Rev 13, 129-145; Mangelsdorf, D. J. et al. (1995) Cell 83, 835-839).

If the central spacing between two ERE half-sites is a critical determinant for the discrimination of an ERE, EBAs should induce gene transcription from only ERE half-sites with three central nucleotides that correspond to the consensus ERE, but not from sequences with other spacing arrangements. To test this prediction, reporter vectors bearing ERE half-sites with various spacing (from 0 to 15 central nucleotides) were generated and examined their responsiveness to the E2-ERα complex, (p65)₂-CDC-(VP16)₂ (FIG. 4B) or (VP16)₂-CDC-(p65)₂. Results revealed that EBAs, just as E2-ERα induced transcription from only ERE half-sites separated by three central nucleotides.

Since 5′-AGGTCATGACCT-′3 is also the consensus palindromic TRE (Freedman, L. P. (1992) Endocr Rev 13, 129-145; Mangelsdorf, D. J. et al. (1995) Cell 83, 835-839), the absence of transcription induction from the reporter construct by either ERα or EBAs also indicates that the functional conservation of sequence discrimination of EREs by ERα is also preserved in the EBAs. Similarly, ERα or EBAs did not induce luciferase activity from the 5′-AGGTCAnAGGTCA-3′, sequence representing the consensus RXRE. In transiently transfected COS-1 cells, ERα, (VP16)₂-CDC-(p65)₂ or (p65)₂-CDC-(VP16)₂ had no effect on luciferase activity from a reporter plasmid bearing one RXRE; whereas RXRα in response to 10⁻⁶ M 9-cis retinoic acid augmented the enzyme activity compared to the control (FIG. 4C).

The inverted 5′-AGAACAnnnTGTTCT-3′ sequence with three non-specific central nucleotides is the consensus RE for GR, AR and PR. If the functional discrimination of half-site sequences were conserved, EBAs, as ERα, should be ineffective as transactivators of the PRE-driven reporter gene from the TATA box promoter. Indeed, in COS-1 cells transfected with an expression vector bearing none (V), a cDNA for PR, ERα, (V16)₂-CDC-(p65)₂ or (p65)₂-CDC-(VP16)₂ together with a reporter plasmid bearing a single PRE, only PR enhanced the reporter enzyme activity when cells were treated with 10⁻⁸ M Progesterone (FIG. 4D).

These results collectively show that the specificity of the finctional discrimination of responsive elements by ERα is also conserved in EBAs.

(9) The Effects of EBAs on Transcription from Estrogen Responsive Gene Promoter Constructs and on the Cell Cycle Progression in Transfected ER-Negative and -Positive Breast Cancer Cell Lines.

EBAs potently modulated transcription from heterologous promoters that rely on a synthetic upstream ERE sequence. However, the temporal regulation of E2 responsive gene expression is the result of small changes in the combination and/or concentration of transcription factors that bind to specific responsive elements within the promoter/enhancer region of a target gene. These factors act synergistically or antagonistically with E2-ER that interacts with a single or multiple EREs flanked by various responsive elements. For example, the pS2 gene confers ER responsiveness through the 5′-GGTCAcggTGGCC-3′ sequence. Positioned between −494 to −393 from the transcription initiation site, this ERE is flanked by AP-1, AP-2, CAAT and GC-Boxes to modulate the expression of the gene (Nunez, A. M. et al. (1989) EMBO J 8, 823-829). Similarly, C3 is also responsive to stimulation by E2, albeit in a cell-context dependent manner (Yi, P. et al. (2002) Mol Endocrinol 16, 1810-1827; Sundstrom, S. A. et al. (1990) Endocrinology 126, 1449-1456; Tzukernan, M. T. et al. (1994) Mol Endocrinol 8, 21-30). The E2-ER mediation of the C3 gene transcription requires the presence of 5′-GGTGGcccTGACC-3′ sequence surrounded by the regulatory elements for CAAT binding protein, interferon-γ, interleukin-6, nuclear factor KB and thyroid hormone (Tzukerman, M. T. et al. (1994) Mol Endocrinol 8, 21-30). The expression of human oxytocin gene confers E2 responsiveness through a variant 5′-GGTGAcctTGACC-3′ ERE sequence flanked by a retinoic acid responsive element and several AP-1, SP-1 and CAAT box regulatory elements (Richard, S. et al. (1990) J Biol Chem 265, 6098-6103) (Richard, S. et al. (1991) J Biol Chem 266, 21428-21433) (Inoue, T. et al. (1994) J Biol Chem 269, 32451-32456)). To ensure that EBAs activate transcription of these ERE-containing genes, promoter constructs were generated and tested their responsiveness to EBAs in transfected MDA-MB-231 cells (FIG. 5A). While CDC had little effect on the reporter enzyme levels, ERα, only in the presence of 10⁻⁹ M E2, p65-CDC-VP16 and (p65)₂-CDC-(VP16)₂ enhanced the enzyme activity from all reporter constructs, the extent of which was dependent upon the promoter-type. The DNA binding defective mutants C*DC* and p65-C*DC*-VP16 had no effect on enzyme levels. These results indicate that the induction of transcription is dependent upon the interaction of transactivators with ERE sequences.

A number of E2 responsive genes including collagenase 1 (Col) and retinoic acid receptor-α (RARα) are regulated by ERE-independent, DNA-dependent ligand-ER signaling (Kushner, P. J. et al. (2000) J Steroid Biochem Mol Biol 74, 311-317; Safe, S. (2001) Vitam Horm 62, 231-252). The tethering of the activation domains of ERs to the jun/fos family of proteins bound to the AP-1 site in the Col promoter provides the responsiveness to E2 (Kushner, P. J. et al. (2000) J Steroid Biochem Mol Biol 74, 311-317). Similarly, the finctional interaction of the activation domains of ERα, but not ERβ, with the Sp-1 transcription factor bound to the GC box provides the E2 responsiveness for the modulation of RARα gene expression (Safe, S. (2001) Vitam Horm 62, 231-252). A reporter construct that contains the human Col or RARα promoter was engineered as a model for the ERE-independent and DNA-dependent signaling pathway. No effect was observed of ERα, CDC or an EBA in the absence or presence of E2 on the luciferase activity driven by the RARα gene promoter (see below) in transiently transfected MDA-MB-231 cells (FIG. 5B). On the other hand, the un-liganded ERα enhanced and the E2-bound ERα repressed the enzyme activity from Col promoter construct. CDC, p65-CDC-VP16 or (p65)₂-CDC-(VP16)₂ had no effect on enzyme levels in the absence or presence of E2. These results collectively indicate that EBAs specifically target ERE-containing genes.

Previous studies showed that the ectopic expression of ERα or ERβ inhibits cell proliferation (Garcia, M. et al. (1992) Proc Natl Acad Sci USA 89, 11538-11542; Wang, M. et al. (1997) Breast Cancer Res Treat 44, 145-151; Wang, M. et al. (1997) Breast Cancer Res Treat 44, 145-151) by arresting the cell cycle at the G1/S phase transition in MDA-MB-231 cells (Lazennec, G. et al. (1999) Mol Endocrinol 13, 969-980; Lazennec, G. et al. (1999) Mol Cell Endocrinol 149, 93-105; Lazennec, G. et al. (2001) Endocrinology 142, 4120-4130). Although it is clear that the restoration of some aspects of hormone dependency in ER-negative breast cancer cells alters the cell proliferation, the underlying mechanisms remain to be elucidated. Since the mediation of cell proliferation by E2-ER could involve multiple pathways, that the activation of ERE-containing genes by EBAs could account, at least in part, for the repression of the cell cycle progression in MDA-MB-231 cells was addressed. To accomplish this, a vector that bears two expression cassettes for the independent synthesis of the enhanced green fluorescent protein (EGFP) and ERα, CDC or an EBA was engineered. The vector was transiently transfected into MDA-MB-231 cells. Cells were subjected to a fluorescence-activated cell sorting (FACS) to separate EGFP-positive cell population, as described (Shang, Y. et al. (2000) Cell 103, 843-852). The cell cycle analysis was simultaneously performed with the EGFP-positive cell population. It was observed that while CDC decreased, E2-ERα, p65-CDC-VP16 or (p65)₂-CDC-(VP16)₂ increased cell population accumulated in the G1 phase. On the other hand, the ERE binding defective mutants, shown for p65-C*DC*-VP16, had no effect on G1/S phase transition. Consistent with previous studies (Garcia, M. et al. (1992) Proc Natl Acad Sci USA 89, 11538-11542; Wang, M. et al. (1997) Breast Cancer Res Treat 44, 145-151; Lazennec, G. et al. (1999) Mol Endocrinol 13, 969-980; Lazennec, G. et al. (1999) Mol Cell Endocrinol 149, 93-105; Lazennec, G. et al. (2001) Endocrinology 142, 4120-4130; Wang, W. et al. (1997) Mol Cell Endocrinol 133, 49-62), these results further implicate ERE-containing genes in cell proliferation. The opposite effect of CDC compared to those of EBAs and E2-ERα, on the G1/S phase transition also indicates a different mechanism of action on cell proliferation.

An effective ERE targeting by EBAs was also observed in the ER-positive, hence hormone responsive, MCF-7 cells derived from human breast adenocarcinoma. MCF-7 cells were grown for three days in a medium that contains charcoal-dextran treated FBS to remove E2 and hence to minimize its effects on endogenous ER. In transiently transfected MCF-7 cells, the 10⁻⁹ M E2 treatment enhanced reporter enzyme activity from the pS2, C3 and Oxytocin promoter constructs (FIG. 6A). Similarly, p65-CDC-VP16 or (p65)₂-CDC-(VP16)₂ potently increased luciferase activity in the absence or presence of 10⁻⁹ M E2 from all constructs. CDC repressed both the basal and E2-induced transcription. As observed in MDA-MB-231 cells, DNA binding defective mutants did not alter enzyme levels in the absence or presence of E2.

CDC or EBAs did not alter the transcriptional response from the RARα or Col promoter whether or not the cells were treated with 10⁻⁹ M E2, whereas the E2 treatment enhanced the reporter activity from the RARα, but not the Col, promoter (FIG. 6B).

In contrast to MDA-MB-231 cells, the 10⁻⁹ M E2 treatment, as shown previously (Lazennec, G. et al. (1999) Mol Endocrinol 13, 969-980; Sutherland, R. L. et al. (1983) Eur J Cancer Clin Oncol 19, 307-318; Osbome, C. K. et al. (1984) Cancer Res 44, 1433-1439; Zajchowski, D. A. et al. (1993) Cancer Res 53, 5004-5011; Foster, J. S. et al. (2001) Trends Endocrinol Metab 12, 320-327), and EBAs enhanced the G1/S phase transition in MCF-7 cells (FIG. 6C). CDC, on the other hand, repressed the cell cycle progression as assessed by an increase in the cell population accumulated in the G1 phase. CDC effectively competed with ERα for binding to ERE in vitro (FIG. 11) and repressed the E2-induced transactivation in situ (FIG. 6A). It is therefore likely that the CDC-mediated suppression of the cell cycle progression in the absence of exogenously added E2 is due to the prevention of the ERE-bound ER function activated by trace amounts of E2 and/or other serum components in the culture medium. To address this issue, MCF-7 cells were cultured in a medium that contains untreated FBS. Cells were then transiently transfected with CDC or C*DC*. CDC dramatically increased the number of cells accumulated in the G1 phase, whereas C*DC* had no effect. Similarly, the treatment of cells with 10⁻⁷ M 4-hydroxytamoxifen (4-OHT), an anti-estrogenic compound in ER-positive breast cancer cells (Jensen, E. V. (1996) Annals of the New York Academy of Sciences 784, 1-17; Jordan, V. C. et al. (1999) Endocr Rev 20, 253-278; McDonnell, D. P. et al. (1999) Trends Endocrinol Metab 10, 301-311), increased the cell population in the G1 phase. These results are consistent with the prediction that the ability of CDC to compete with ERα for an ERE is responsible for the repressive effect of the protein on the cell cycle progression mediated by the E2-ER complex. Thus, these findings further confirm the conclusion that the ERE-containing gene network is involved in cell cycle regulation, and indicate that EBAs specifically target ERE sequences of the estrogen responsive genes even in the presence of an endogenous E2-ER signaling.

c) Discussion

The modular nature of ER allowed us to initially engineer a monomeric ERE binding protein by cojoining two C domains with the D domain. The integration of increasing numbers of VP-16 and/or of p65 ADs into this module generated constitutively active EBAs. These transactivators specifically targeted ERE sequences and robustly activated ERE-containing genes. The activation potentials of these proteins were independent of ligand, ER-subtype, promoter- and cell-context.

E2 is important for the growth and development of the mammary tissue (Jensen, E. V. (1996) Annals of the New York Academy of Sciences 784, 1-17; Jordan, V. C. et al. (1999) Endocr Rev 20, 253-278; Russo, J. et al. et al. (2000) J Natl Cancer Inst Monogr, 17-37). E2 is also involved in the proliferation and differentiation of ER-positive mammary carcinoma cells (Jensen, E. V. (1996) Annals of the New York Academy of Sciences 784, 1-17; Jordan, V. C. et al. (1999) Endocr Rev 20, 253-278; Russo, J. et al. et al. (2000) J Natl Cancer Inst Monogr, 17-37). A complex array of convergent and divergent mechanisms appear to be involved in the mediation of E2-ER signaling at both genomic and non-genomic levels (Parker, M. G. (1998) Biochem Soc Symp 63, 45-50; McKenna, N. J. et al. (1999) Endocr Rev 20, 321-344; Hall, J. M. et al. (2001) J Biol Chem 276, 36869-36872). The genomic effect of E2-ER signaling is mediated through ERE-dependent and -independent pathways. These events are responsible for the expression of a number of genes involved in the cellular metabolism, mitogenesis, morphogenesis, motogenesis and apoptosis (Charpentier, A. H. et al. (2000) Cancer Res 60, 5977-5983; Rey, J. M.et al. (2000) J Mol Endocrinol 24, 433-440; Soulez, M. et al. (2001) J Mol Endocrinol 27, 259-274; Gruvberger, S. et al. (2001) Cancer Res 61, 5979-5984; Inoue, A. et al. (2002) J Mol Endocrinol 29, 175-192). However, only a few genes identified to date are shown to be mediated by a direct ER-ERE interaction (Klinge, C. M. (2001) Nucleic Acids Res 29, 2905-2919). The ERE binding activators that target only ERE sequences altered the G1/S phase transition in both ER-negative MDA-MB-231 and ER-positive MCF-7 cells in a manner similar to the E2-ERα complex. Consistent with previous studies (Garcia, M. et al. (1992) Proc Natl Acad Sci USA 89,11538-11542; Wang, M. et al. (1997) Breast Cancer Res Treat 44, 145-151; Lazennec, G. et al. (1999) Mol Endocrinol 13, 969-980; Lazennec, G. et al. (1999) Mol Cell Endocrinol 149, 93-105; Lazennec, G. et al. (2001) Endocrinology 142,4120-4130; Wang, W. et al. (1997) Mol Cell Endocrinol 133, 49-62), the results disclosed herein further indicate the critical importance of ERE-containing genes in the regulation of cell proliferation. The implications are that these transactivators can be utilized in the identification of only ERE-containing E2 responsive genes independent of ligand, ER status and subtype, promoter- and ceH-context. This allows for better understanding of the differences in the mechanisms of genomic responses, which present a remarkable phenotypic diversity, among breast tumors. This understanding can aid the development of strategies for breast cancer treatment. The suppression of ERE-containing genes, for example, by the designer ERE-binding repressors can constitute a therapeutic approach for ER-positive tumors. Whereas, the activation of the ERE-containing gene network by EBAs can provide an effective strategy for the treatment of ER-negative neoplasms.

The understanding that estrogens are involved in the initiation and development of estrogen target-tissue cancers has led to the development of compounds with diverse pharmacology that specifically. target ER, hence are ER-ligands (Jordan, V. C. et al. (1999) Endocr Rev 20, 253-278; McDonnell, D. P. et al. (1999) Trends Endocrinol Metab 10, 301-311). ER-ligands display agonistic and/or antagonistic properties depending upon the ER subtype, promoter- and cell-context. The binding of an agonist to ER induces conformational changes that allow the complex to interact with EREs. The agonist- and ERE-bound ER recruits co-regulatory proteins which promote remodeling of chromatin and forming a transcription complex. In contrast, antagonists induce conformational changes in ER that differ from those induced by E2. This prevents ER from interacting with co-activators, instead may promote interaction with co-repressors (Shang, Y. et al. (2000) Cell 103, 843-852; Lavinsky, R. M. et al. (1998) Proc Natl Acad Sci USA 95, 2920-2925), and subsequently inhibits the ability of ER to induce transcription (Jensen, E. V. (1996) Annals of the New York Academy of Sciences 784, 1-17; Jordan, V. C. et al. (1999) Endocr Rev 20, 253-278; McDonnell, D. P. et al. (1999) Trends Endocrinol Metab 10, 301-311). This constitutes the molecular basis of anti-estrogen therapy. However, the eventual circumvention of the beneficial effects of ligand-based treatments together with the unintended development of other estrogen target tissue malignancies has been a major concern for endocrine approaches in the treatment of breast cancer.

In addition to ER-ligands, the co-expression of variant ERs with dominant-negative phenotype has been proposed to be an effective modality in the functional inactivation of wild-type (WT)-ERs and subsequent inhibition of tumor growth (Muyan, M. et al. (2001) Mol Cell Endocrinol 182, 249-263),(Elliston, J. F. et al. (1990) J Biol Chem 265, 11517-11521; Wang, Y. et al. (991) Mol Endocrinol 5, 1707-1715; Ince, B. A. et al. S. (1993) J Biol Chem 268, 14026-14032; Schodin, D. J. et al. (1995) J Biol Chem 270, 31163-31171; Ma, Z. Q. et al. (999) J Steroid Biochem Mol Biol 69, 155-163). Although multiple pathways are involved, the extent of heterodimerization of a variant with the WT-ER is thought to correlate with the strength of functional interference (Ince, B. A. et al. S. (1993) J Biol Chem 268, 14026-14032; Schodin, D. J. et al. (1995) J Biol Chem 270, 31163-31171; Ma, Z. Q. et al. (1999) J Steroid Biochem Mol Biol 69, 155-163). It is expected that the heterodimerization leads to the formation of transcriptionally inactive complexes (Ince, B. A. et al. S. (1993) J Biol Chem 268, 14026-14032; Schodin, D. J. et al. (1995) J Biol Chem 270, 31163-31171; Ma, Z. Q. et al. (1999) J Steroid Biochem Mol Biol 69, 155-163). However, heterodimerization also provides characteristics to ER by combining two distinct functional properties of the contributing partners (Muyan, M. et al. (2001) Mol Cell Endocrinol 182, 249-263). This combinatorial diversity, hence heterogeneity, of ERs is an obstacle for the use of variants as an effective means of regulating estrogen signaling.

The ERE binding proteins disclosed herein preclude the dependency on ligand for function and also the limitation of monomer association into biologically active dimmers. This generates a homogeneous population of monomeric ERE binding proteins with constitutive activity. Moreover, it was observed that the potent activities of EBAs to regulate the transcription of ERE-containing genes were preserved in various cell types even in the presence of a functional E2-ER signaling, as observed in MCF-7 cells. This presents an opportunity for the development of an inducible system that controls the expression of ERE binding proteins with desired biopotency at any target cell. This can serve as a basis to alter the growth of breast tumors independent of ER status. Furthermore, in contrast to antiestrogen therapy, such constructs can be targeted to specific tissues of interest by gene therapy approaches.

One of the most common features of transcription factors is the Cys2-His2 zinc-finger (C₂H₂ZF) domain that functions as the DNA binding module. Each C₂H₂ZF of a transfactor recognizes a three-nucleotide site with relative independence from adjacent nucleotides. Based on this DNA sequence recognition property, a covalent conjugation of various synthetic C₂H₂ZF components in tandem produces a binding module with remarkable specificity for a pre-determined asymmetric DNA sequence (Pabo, C. O. et al. (2001) Annu Rev Biochem 70, 313-340; Beerli, R. R. et al. (2002) Nat Biotechnol 20, 135-141). Genetic joining of an effector (activation or repression domain) to a C₂H₂ZF module has generated transcription factors for the regulation of expression of specific genes (Zhang, L. et al. (2000) J Biol Chem 275, 33850-33860; Liu, P. et al.(2001) J Biol Chem 276, 11323-11334; Beerli, R. R. et al. (2002) Nat Biotechnol 20, 135-141; Kim, J. S. et al. (1997) Proc Natl Acad Sci USA 94, 3616-3620; Kang, J. S. et al. (2000) J Biol Chem 275, 8742-8748). C₂H₂ZF based transregulators are powerful tools for improving the understanding of a specific gene function through transcriptional activation/repression. They are also valuable for gene therapy wherein the potentiation or attenuation of a single gene function can have therapeutic benefits. However, the requirement for a predetermined DNA sequence necessitates the engineering of a C₂H₂ZF-transregulator for each distinct sequence. This limits the identification of genes driven by variant regulatory sequences that are responsive to the same transcription factor. Here, advantage was taken of the intrinsic specificity and flexibility of DNA sequence decoding properties of the C domain of ERα and generated EBAs with gradient biopotencies that specifically and robustly activate various ERE sequence-containing genes. This provides a means to simultaneously regulate multiple genes responsible for a phenotype, as in breast cancer. This approach can be extended to members of multi-subunit complexes of the steroid/thyroid hormone receptor superfamily and non-ligand dependent transcription factors, such as jun/fos family, which operate primarily as homo- and/or hetero-dimmers. Targeted regulation of endogenous genes by specific responsive element binders with activator or repressor functions can provide alternative/additional paradigms to experimental biology and medicine.

EBAs induce gene transcription synergistically. An increase in the number and/or diversity of ADs allowed an EBA to induce synergy from a single ERE, hence the AD-dependent synergy. Also found was that an EBA retained its ability to induce synergy from tandem ERE sequences compared to a single ERE, the ERE-dependent synergy. Transcription is a dynamic event in which sequential cycles of association and dissociation of co-factor/mediator complexes regulate transcription of target genes through acetylation/de-acetylation processes and subsequent modifications of local chromatin architecture (Chen, H. W. et al. (1999) Cell. 98, 675-686). The synergy mechanism can involve an increase in the local concentrations of a co-factor/mediator that interacts with multiple copies of a single AD species bearing EBA bound to an ERE, or diverse co-factors associated with an EBA with different ADs. Co-regulators with different histone modifying activities show different preferences for free histones and distinct targets within the histone substrates (Strahl, B. D. et al. (2000) Nature 403, 41-45). An increase in the concentration of a co-factor(s) can form a scaffold necessary for an inter-molecular interaction that stabilizes the transcription complex at the promoter or can modify local chromatin architecture extensively. Either or both events can lead to an extended transcriptional initiation and re-initiation and subsequent synergy. Similar mechanisms can also be responsible for the synergy observed with tandem ERE sequences. The capacity of the EBA proteins to activate gene transcription with a gradient as a result of an increase in the number and/or diversity of ADs allows mechanisms of transcription in general and of transcriptional synergy by defined components in experimental systems to be addressed. cl Example 2

Expression of estrogen responsive genes in a spacio-temporal manner is the result of small changes in the combination and/or concentration of transcription factors that bind to specific response elements within a promoter/enhancer region of the target gene. These factors act synergistically or antagonistically with the E2-ER complex that interacts with a single or multiple estrogen responsive elements (EREs) among binding sites for other transcription factors. For example, the expression of Cathepsin D (CatD), a lysosomal proteinase, is induced by estrogen in mammary cancer cells through a non-consensus ERE surrounded by several AP-1, Sp1 and MLPE response elements in the proximal enhancer region of the gene (Augereau P. et al. Mol Endocr 8:693-703; Cavailles V. et al. (1993) Proc Natl Acad Sci USA 90:203-7). ERE sequences and the other response elements acting in concert are required for the estrogen responsiveness (Augereau P. et al. Mol Endocr 8:693-703; Cavailles V. et al. (1993) Proc Natl Acad Sci USA 90:203-7).

In addition to non-hormonal transcriptional elements, presence of both ERE and Progesterone response element (PRE) within the proximal promoter of the same gene can permit the convergent as well as divergent regulation by E2 and Progesterone (Kraus W.L. et al. (1993) Endocrinology 132:2371-9; Katzenellenbogen BS (2000) J Soc Gynecol Investig 7:S33-7). While ERE and PRE act synergistically to enhance the transcription of vitellogenin gene II, PRE in the promoter region of the human c-fos gene antagonizes the activity of ERE. Thus, although the E₂-ER-ERB complex plays a critical role in the modulation of estrogen responsive genes as an enhancer, combinatorial effects of many regulatory components specific to each gene ultimately determine the tining, direction and magnitude of expression. This contrasts to experimental systems wherein the transcriptional activity of a reporter gene is solely dependent on ER as a transcription regulator. Therefore, an altered state of different signaling pathways converging onto an estrogen-responsive gene, as in CatD and C3, can effectively-circumvent the ERE-modulated transcription. Since both anti-estrogens and dominant-negative receptor variants target ER-mediated signaling, aberrant signaling pathways converging onto ER and/or ER-mediated events can circumvent the regulatory potential of the ligand-ER. This, in turn, can contribute to tumor progression and acquisition of resistance to anti-estrogen. This is also consistent with recent reports that a dominant negative ERα that bears a potent suppressor does not alter the basal transcriptional activity of the endogenous estrogen responsive genes but it decreases E2-augmented expression, presumably as a result of heterodimerization with the WT receptor (de Haan G. (2000) J Biol Chem 275:13493-501).

Since the ERE binding protein serves as an effective platform for the generation of potent molecular “on” switches for ERE-driven genes independent of ligand, dimerization, ER subtype, promoter- and cell-context, it was reasoned that CDC can also be used to generate potent “off” switches for the expression of these genes. This would provide an active repression of the responsive genes in contrast to the prevention of ER-mediated transactivation by antiestrogenic compounds.

The acetylation-state of chromosomal histones correlates with transcriptional status. Hyperacetylated regions of chromatin frequently contain active transcription units, while hypoacetylated chromatin is transcriptionally silent. The enzymatic activities of both histone acetyl transferases (HATs) and histone deacetylases (HDACs) ultimately responsible for the relative levels of histone acetylation, hence the extent and direction of gene transcription (Struhl K (1998) Genes & Dev 12:599-606; Strahl, B. D. et al. (2000) Nature 403, 41-45).

The Krüppel associated box (KRAB) domain is an amino acid sequence motif found at the amino-terminus of nearly one-third of all Krüppel/TFIIIA-type C2H2 zinc finger proteins (Bellefroid EJ. et al. (1991) Proc Natl Acad Sci USA 88:3608-12). This highly conserved domain displays potent, DNA-binding dependent repression of transcription that requires the KRAB-associated protein-1 (KAP-1) as the co-repressor (Margolin JF et al. (1994) Proc Natl Acad Sci USA 91:4509-13; Friedman J R et al. (1996) Genes Dev 10:2067-78; Moosmann P. et al. (1996) Nucleic Acids Res 24:4859-67; Moosmann P. et al. (1997) Biol Chem 378:669-77). The KAP-1 co-repressor in turn recruits the nucleosome remodeling histone deacetylase complex (NuRD). The NuRD complex is composed of three functionally distinct proteins, Mi-2, MTA-2 and MBD3 that assemble through multiple inter-molecular interactions to form the complex. Mi-2 possesses an ATP-dependent chromatin remodeling activity and anchors the NuRD complex to the core HDAC1 and HDAC2 complexes that deacetylate histones. The methyl-DNA-binding protein MBD3, on the other hand, appears to mediate the ability of NuRD to repress the transcription in a methylation-dependent manner. The subsequent interaction of the NuRD-HADC complex with HP1, a family of nonhistone heterochromatin-associated proteins, results in the formation of a heterochromatin-like complex, leading to gene silencing. All members of the KRAB domains repress transcription when tethered to DNA. Fusions of the KR-AB domain derived from KOX1/ZNF10 zinc finger protein to the DNA binding domains of both LexA and Ga14 factors, for example, effectively silence the transcription of reporter genes in transfected cells regardless of the distance of the cognate binding element to the transcription initiation site. In addition to the recruitment of co-repressor complexes by the fusion proteins to the promoter, specific inhibition of some component(s) of RNA polymerase II and III transcription by KRAB appears also involved in transcription suppression (Moosmann P. et al. (1996) Nucleic Acids Res 24:4859-67; Moosmann P. et al. (1997) Biol Chem 378:669-77).

Transcriptional repression by the basic helix-loop-helix zipper protein Mad1 requires DNA binding as a ternary complex with its heterodimer partner Max and the mammalian co-repressor protein Sin3. Similarly, the repression of thyroid hormone responsive genes is mediated by the recruitment of Sin3 repressor complex by the unliganded TR-corepressor N-CoR/SMRT complexes. The Sin3 complex contains two additional proteins, SAP18 and SAP30. These proteins are integral components of the Sin3 complex. They appear involved in the facilitation or stabilization of co-repressor interactions with Sin3 and HDACs. The interaction between Mad1 and Sin3 is mediated by a 35 amino-acid region in the amino-terminus of Mad1 (Sin3 interaction domain, or SID) (Kasten MM. et al. (1996) Mol Cell Biol 16:4215-21; Ayer DE et al. (1996) Mol Cell Biol 16:5772-81). The tethering of SID, just as KRAB, to a DBD of a transcription factor suppresses gene transcription mediated by the response element to which the parent transcription factor binds. As NuRD complex, Sin3 or SID-mediated transcriptional repression involves HDAC1 and HDAC2 recruitment to the promoter. Thus, although distinct set of transcriptional repressors recruits different co-repressor complexes to establish a represosome, the recruitment of HADC1 and HDAC2 by NuRD or Sin3 complex is critical for the active gene repression (Kasten MM. et al. (1996) Mol Cell Biol 16:4215-21; Ayer DE et al. (1996) Mol Cell Biol 16:5772-81).

Since the tethering of a single or multiple copies of the same or different ADs to CDC generated EBAS, it was reasoned that fusion of KRAB and/or SID as a single or multiple copies to CDC should produce ERE-binding repressors (EBRs) that effectively suppress transcription of estrogen responsive genes.

a) Results and Discussion
(1) Engineering and Functional Analysis of ERE Binding Repressors.

To engineer potent repressors of ERE containing genes, the cDNA of the repressor domain KRAB domain (RD) of the KOX-1 protein (Aronica, S. M. et al. (1993) Mol Endocrinol 7, 743-752) to CDC was generated. This KRAB CDNA encompasses residues 1-90 and was produced by PCR using primers based on the published cDNA sequence of KOX1 (Thiesen HJ (1990) New Biol 2:363-74) and a human testis cDNA library (Clontech Laboratories, Palo Alto, Calif.) as the template. Similarly, the cDNA for SID that encodes residues 1-35 of Mad1 (Ayer DE et al. (1996) Mol Cell Biol 16:5772-81) was generated by PCR from the testis cDNA library. The CDNA for KRAB or SID as a single copy was genetically fused to the amino- (KRAB-CDC or SID-CDC) or carboxyl-terminus (CDC-KRAB or CDC-SID) of CDC to generate EBRs.

The effectiveness of EBRs to suppress transcription was then assessed in ER-negative COS-1 or CHO cells. Cells were transiently transfected with an expression vector without or with a cDNA for ERα or for an EBR, together with a reporter plasmid beating none-or one consensus ERE sequence juxtaposed to the complex SV40 promoter that drives the expression of firefly luciferase cDNA as the reporter. Cells were also transfected with a plasmid bearing a cytomegalovirus (CMV) promoter driving the expression of the renilla luciferase cDNA for monitoring transfection efficiency. Normalized activity from each reporter was compared to the basal activity from the reporter construct bearing no ERE in response to the parent expression vector (V) in the absence of ligand, which was set to one.

Results revealed that expression of ERα or EBRs had no effect on gene transcription from the SV40 promoter without an ERE. Similarly ERα had little effect on the enzyme activity in the absence or presence of 10⁻⁹ M E2 or 10⁻⁶ M 4-OHT from the SV40 promoter with one juxtaposed ERE. On the other hand, CDC unexpectedly suppressed basal transcription about 50% in COS-1 cells whether or not cells were treated with E2 or 4-OHT. This contrast to CHO cells in which CDC had little effect on enzyme activity in the absence or presence of E2 or 4-OHT.

EBRs with one RD specie at either terminus dramatically repressed the reporter enzyme activity in COS-1 and CHO cells. While KRAB-CDC or SID-CDC decreased the SV40-induced transcription about 50% of the basal transcription, the relative repression reached to 80% of the basal enzyme activity with CDC-KRAB or CDC-SID. A stronger transcription repression mediated by KRAB or SID positioned at the carboxyl-terminus of CDC was observed with KRAB or SID placed at the amino-terminus. This indicates that RDs are more effective in repressing transcription when placed at the carboxyl-terminus of CDC. These together with the observations that EBR had no effect on the reporter gene expression from SV-40 promoter bearing no ERE indicate that the ability of an EBR to repress transcription is dependent upon binding to an ERE and the presence of an RD. Thus, as with the development of molecular “on” switches, the genetic fusion of a RD to the ERE binder CDC is also a feasible paradigm in engineering of “off” switches for the ERE-containing genes.

(2) Effects of Increasing Number and/or Diversity of RD on the Ability of an EBR to Repress Transcription in Various Cells

Since an EBR with a single copy of a RD placed at either terminus repressed the transcription about 80% of that observed with the parent vector, it was asked whether an increase in the copy number of a RD further augments the biopotency of the repressor. To address this point, the cDNA of a RD was fused to both termini. KRAB-CDC-KRAB or SID-CDC-SID further suppressed the reporter enzyme activity compared to an EBR bearing single RD at either domain. Thus, increasing the number of a RD provides the EBA with more transcriptional strength. Interestingly, EBRs bearing KRAB at either or both termini are more potent repressor than those with the SID.

The initial events in the establishment of a functional represosome by KRAB and SID involve the recruitment of distinct sets of co-repressor complexes. Integration of these complexes recruited can provide an EBR with stronger transcription-repression potency compared to those with single RD specie. Indeed, KRAB-CDC-SID or SID-CDC-KRAB repressed the transcription more potently compared to that observed with KRAB-CDC-KRAB or SID-CDC-SID; the repression by either construct reached to 95% of that observed in response to the parent vector.

Similarly, EBRs effectively repressed transcription of the reporter gene in ER-negative CHO cells. Thus, EBRs with diverse RDs can be used as potent repressors of ERE-containing genes regardless of ligand, ER subtype and cell-context.

(3) Effects of EBRs on the Expression of Estrogen Responsive Genes.

EBRs are potent repressors of transcription from a strong heterologous promoter, which rely on an upstream consensus ERE sequence. However, most of the estrogen responsive genes bear a single or multiple ERE sequences that deviate from the consensus with one or more nucleotides. Moreover, as mentioned above, the direction and magnitude of estrogen responsive gene expression are dictated not only by the E2-ER complex but also by the integrated actions of trans-acting proteins. The type and availability of these protein components specific to each gene act cooperatively or antagonistically with the E2-ER complex to modulate the gene expression. To ensure that EBRs repress transcription of estrogen responsive genes as well, reporter constructs were engineered bearing the enhancer/promoter region of estrogen responsive gene CatD or C3 that derives the expression of the luciferase cDNA were generated. The expression of CatD is augmented by estrogens through a non-consensus ERE core (5′-GGCCGgcTGACC-3′, deviations are underlined) located between −271 to −259 from the transcription initiation site. This ERE is flanked by several AP-1, Sp1 and MLPE response elements (Augereau P. et al. Mol Endocr 8:693-703). The ERE sequence is shown to be required for the estrogen responsiveness, albeit modestly (0.6-0.9 fold compared to the basal expression) (Augereau P. et al. Mol Endocr 8:693-703; Miralles F. et al. Biochem Biophy Res Comm 203:711-8). Similarly, C3 is also responsive to stimulation by both estrogens and tamoxifen, or its more active analog 4 hydroxy-tamoxifen (4-OHT) (Sundstrom, S. A. et al. (1990) Endocrinology 126, 1449-1456; Tzukerman, M. T. et al. (1994) Mol Endocrinol 8, 21-30). Tamoxifen and 4-OHT are the widely used antiestrogenic compounds in the treatment of breast cancer. The E2 mediation of transcription requires the presence of a non-consensus ERE, 5′-GGTGGcccTGACC-3′ surrounded by regulatory elements for CAAT binding protein, interferone-γ, interleukin-6, nuclear factor κB, glucocorticoid and thyroid hormones (Tzukerman, M. T. et al. (1994) Mol Endocrinol 8, 21-30).

The CatD and C3 promoters were generated by PCR using pCD3542 (Augereau P. et al. Mol Endocr 8:693-703) and pC3TI (Fan JD. (1996) Mol Endocrinol 10: 1605-16) reporter plasmids as templates, respectively. A fragment of 730 bp [−750 to −22, +1 is referred to the first ATG, (Parker, M. G. (1998) Biochem Soc Symp 63, 45-50)] of the promoter region of CatD gene or 580 bp [−516 to +61, +1 as the transcription start site) of the C3 gene (Tzukerman, M. T. et al. (1994) Mol Endocrinol 8, 21-30)] was generated. PCR fragments were then inserted into the reporter plasmid pGL3-Basic linearized with appropriate restriction enzymes.

COS-1 cells were transfected with a reporter construct bearing CatD or C3 promoter together with the expression vector bearing none (V, as control), cDNA for ERα or an EBR. Following a transfection, cells were treated without or with 10⁻⁹ M E2 or 10⁻⁷ M 4-OHT for 24 h.

Results revealed that ERα had no effect on the enzyme activity in the absence of ligand, while the E2-ERα complex enhanced the transcription, albeit modestly, compared to the control. 4-OHT displays also partial agonistic effect on ERα-mediated transcriptional responses in a promoter-dependent manner. The 4-OHT partial-agonism is manifested as transcriptional responses that are substantially lower than those observed with E2. 4-OHT did not alter the transcription of the reporter enzyme mediated by ERα, while 4-OHT at the same concentration prevented the E2-ERα complex-mediated augmentation on the enzyme activity. CDC alone had no effect on enzyme activity whether or not cells were treated with ligands. In contrast to effects observed on the SV-40 promoter bearing one ERE, KRAB at either or both termini of CDC did not alter the transcription of the reporter enzyme compared to the control levels. On the other hand, CDC with a SID fused to either terminus or both termini decreased the transcription about 60% of that observed with the parent vector. There was no further decrease in the enzyme levels in response the EBR with two RDs (KRAB-CDC-SID or SID-KRAB). This indicates that an EBR with SID, but not KRAB, fused to both domains is an effective repressor of the CatD promoter-mediated transcription.

When transcriptional responses were tested in transfected cells with C3 promoter construct, it was observed that ERα had increased the enzyme activity about two-fold compared to the parent vector control (V) in the absence of ligand, while the E2-ERα complex enhanced the transcription about 11-fold. The 4-OHT-ERα complex also augmented the reporter enzyme about three-fold. This contrasted to the response observed with the CatD promoter, whose activity was not altered by the 4-OHT bound ERα. 4-OHT on the other hand, effectively decreased the transcription induction from the C3 promoter mediated by the E2-ERα complex. As observed with the CatD promoter, KRAB fused to either terminus or both termini of CDC had no effect on the reporter enzyme activity in the absence or presence of ligand. Whereas, SID as a single copy fused to either terminus or both termini of CDC repressed the reporter enzyme activity substantially. Interestingly, EBR bearing both RDs as a single copy at either terminus (KRAB-CDC-SHD or SID-CDC-KRAB) had similar repression biopotency compared to that observed with SID-CDC-SID.

These results confirm the contention that antiestrogenic compounds target solely ER-mediated transcriptional events without altering the basal transcription and indicate that EBRs can be used as potent repressors of ERE-containing genes regardless of ligand and ER-subtype, promoter- and cell-type.

(4) Infectivity of Model Cell Lines with Recombinant Adenovirus

To assess the infectivity of the model cell lines by adenovirus, initially an adenovirus that expresses the β-galactosidase cDNA. Viral plaques were isolated and propagated to produce lysate containing infectious recombinant virus. The recombinant adenovirus at different multiplicities of infection (MOI) was introduced, (number of virus per cell whose titer had been determined with a plaque assay (plaque forming unit, pfu/ml)), for 24 h into ER-negative MDA-MB-231 cells and ER-positive MCF-7 cells. Cells were then-fixed with 1% glutaraldehyde in PBS and in situ β-galactosidase assay was performed as described (Lim, K. et al. (1989) BioTechniques 7, 576-579). Results revealed that both cell-lines were infected with high efficiency, albeit at different MOIs (FIG. 10). MDA-MB-231 cells were infected with 100% efficiency by the recombinant adenovirus at 250 MOI, whereas the MOI for MCF-7 cells was 500. Kinetic studies in both cell lines further showed that the color development, as an indication of the β-galactosidase synthesis, reaches maximum levels at 18 h following infection with an optimal MOI.

Assessing the infectivity of LCC cell lines by adenovirus can be performed to obtain optimal MOIs for subsequent usage.

(5) Testing the Effects of Regulatory Proteins on the Expression of Endogenous E2 Responsive Genes

An effective gene expression/delivery to breast cancer cells allows one to test the prediction that transregulatory proteins specifically and potently regulate the ERE-containing E2-responsive genes in native chromatin context. Among the breast cancer cell lines, MDA-MB-231 cells have been widely used as a model for the regulation of the Catepsin D (CatD) and c-myc gene expression (Lazennec, G. et al. (2001) Endocrinology 142, 4120-4130; Lazennec, G. et al. (1999) Mol. Cell. Endocrinol. 149, 93-105; Jeng, M. H. et al. (1993) Molecular & Cellular Endocrinology 97, 115-123; Jeng, M. H. et al. (1994) Cancer Lett. 82, 123-128). The E2-ERα responsiveness of the CatD gene is mediated by non-consensus EREs in its proximal promoter, whereas, the E2 responsiveness of the c-myc gene is mediated by the ERE-independent pathway. To effectively address whether EBAs activate specifically ERE-driven genes in cells expressing endogenous ERα, we infected MDA-MB-231 cells with adenovirus bearing no (CMV), or p65-CDC-VP16 (PV), cDNA (FIG. 11). In infected cells, both E2-ERα and PV enhanced the transcription of the ERE-driven CatD gene expression′ compared to the parent adenovirus bearing no cDNA (CMV) about 3-fold, while both constructs had no effect on the house keeping gene β-actin mRNA levels, as expected. On the other hand, only E2-ERα, but not PV, repressed MnRNA levels of c-Myc, whose gene expression is regulated by the DNA-dependent and ERE-independent pathway, 3-fold compared to the parent adenovirus. These results are consistent with the expectation that ERE-binding transregulators specifically target ERE-driven endogenous genes. Importantly, as observed previously (Huang, J. et al. (2004) Mol. Cell. Endocrinol. 218, 65-78), PV, as the E2 treatment, dramatically decreased cell population accumulated in G1 phase, shown are cell cycle histograms in FIG. 12. Thus, PV regulates cell cycle proliferation in a manner similar to the E2 treatment in adenovirus infected MDA-MB-231 cells. Importantly, the infection with adenovirus bearing ERE binding defective P*V, had no effect on cell cycle progression (data not shown). Consistent with results disclosed herein, these results collectively show that specific targeting of ERE-driven genes is necessary and sufficient to alter cell proliferation in cell lines that emulate de novo endocrine resistant breast neoplasms. These results also establish the adenovirus gene delivery approach as an effective means in cell culture systems. Thus, these findings provide a basis for the rational that targeted regulation of ERE-driven genes provides a means for the treatment of breast cancers.

These results also indicate the feasibility of testing the effectiveness of transregulators delivered by recombinant adenoviruses in altering experimental tumor growth in animal as translational experimental models.

(6) Tissue -Specific Expression of Transgene Delivered by Adenovirus

The whey acidic protein (WAP) promoter is known to be active in pregnant and lactating mammary epithelial cells as well as mammary tumors of mice (Ozturk-Winder, F. et al. (2002) Cancer Gene Ther. 9, 421-431). Studies have indicated that a proximal 405 base-pair (bp) fragment of the murine WAP promoter confers mammary-specific expression upon a marker gene in transgenic mice (Ozturk-Winder, F. et al. (2002) Cancer Gene Ther. 9, 421-431). These observations together with the findings that infection of a number of established human mammary and nonmammary cell lines with a retroviral vector bearing WAP promoter limit the transgene expression to mammary tumor cell lines indicate that the WAP promoter is active in human mammary cells and mammary tumor cells. These results also imply that the extended proximal WAP promoter can be useful for directing therapeutic gene expression to mammary tumors (Ozturk-Winder, F. et al. (2002) Cancer Gene Ther. 9, 421-431).

To accomplish breast tissue-specific delivery of genes encoding the disclosed transregulatory proteins by recombinant adenoviruses, one can exchange the adenovirus promoter CMV that derives the expression of the transgene cDNA with the 405 bp promoter of the WAP gene using molecular biology approaches we have described (Yi, P. et al. (2002) Mol. Endocrinol. 16, 1810-1827; Muyan, M. et al. (2001) Mol. Cell. Endocrinol. 182, 249-263; Sathya, G. et al. (2002) Mol. Cell. Endocrinol. 192, 171-185). One can then generate the modified adenoviruses bearing our novel transregulators to infect various cell lines derived from breast tissue as well as non-breast tissues. Transregulatory proteins are only expressed in cell lines derived from breast tissue and in experimental breast tumors in animal models.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### G. References

Ali, S., Metzger, D., Bomert, J. M., and Chambon, P. (1993) Embo J 12, 1153-1160
Angel, P., Baumann, I., Stein, B., Delius, H., Rahmsdorf, H. J., and Herrlich, P. (1987) Mol Cell Biol 7, 2256-2266
Aronica, S. M., and Katzenellenbogen, B. S. (1993) Mol Endocrinol 7, 743-752
Arteaga, C. L., and Truica, C. I. (2004) Semin. Oncol. 31, 3-8
Arteaga, C. L., Moulder, S. L., and Yakes, F. M. (2002) Semin. Oncol. 29, 4-10
Augereau P, Miralles F, Cavailles V, Gaudelet C, Parker M, Rochefort H 1994 Characterization of the proximal estrogen-responsive element of human cathepsin D gene. Mol Endocr 8:693-703
Ayer D E, Laherty C D, Lawrence Q A, Armstrong A P, Eisenman R N (1996) Mad proteins contain a dominant transcription repression domain. Mol Cell Biol 16:5772-81
Beerli, R. R., and Barbas, C. F., 3rd. (2002) Nat Biotechnol 20, 135-141
Bellefroid E J, Poncelet D A, Lecocq P J, Revelant O, Martial J A (1991) The evolutionarily conserved Kruppel-associated box domain defines a subfamily of eukaryotic multifingered proteins. Proc Natl Acad Sci USA 88:3608-12
Benecke, A., Chambon, P., and Gronemeyer, H. (2000) EMBO Reports 1, 151-157
Benten, W. P., Stephan, C., Lieberherr, M., and Wunderlich, F. (2001) Endocrinology 142, 1669-1677
Berry, M., Metzger, D., and Chambon, P. (1990) EMBO J. 9, 2811-2818
Brand, N. J., Petkovich, M., and Chambon, P. (1990) Nucleic Acids Res 18, 6799-6806
Brunner, N., Boulay, V., Fojo, A., Freter, C. E., Lippman, M. E., and Clarke, R. (1993) Cancer Res. 53, 283-290
Brunner, N., Boysen, B., Jirus, S., Skaar, T. C., Holst-Hansen, C., Lippman, J., Frandsen, T., Spang-Thomsen, M., Fuqua, S. A., and Clarke, R. (1997) Cancer Res. 57, 3486-3493
Brunner, N., Frandsen, T. L., Holst-Hansen, C., Bei, M., Thompson, E. W., Wakeling, A. E., Lippman, M. E., and Clarke, R. (1993) Cancer Res. 53, 3229-3232
Brzozowski, A. M., Pike, A. C., Dauter, Z., Hubbard, R. E., Bonn, T., Engstrom, O., Ohman, L., Greene, G. L., Gustafsson, J. A., and Carlquist, M. (1997) Nature 389, 753-758
Castles, C. G., Oesterreich, S., Hansen, R., and Fuqua, S. A. (1997) J Steroid Biochem. Mol. BioL 62, 155-163
Catherino, W. H., and Jordan, V. C. (1995) Cancer Lett. 90, 35-42
Cavailles V, Augereau P, Rochefort H (1993) Cathepsin D gene is controlled by a mixed promoter, and estrogens stimulate only TATA-dependent transcription in breast cancer cells. Proc Natl Acad Sci USA 90:203-7
Channon, K. M., and George, S. E. (1997) QJM90, 105-109
Charpentier, A. H., Bednarek, A. K., Daniel, R. L., Hawkins, K. A., Laflin, K. T., Gaddis, S.,
MacLeod, M. C., and Aldaz, C. M. (2000) Cancer Res 60, 5977-5983
Chen, D., Ma, H., Hong, H., Koh, S. S., Huang, S. M., Schurter, B. T., Aswad, D. W., and Stallcup, M. R. (1999) Science 284, 2174-2177.
Chen, H. W., Lin, R. J., Xie, W., Wilpitz, D., and Evans, R. M. (1999) Cell. 98, 675-686
Chen, Z., Yuhanna, I. S., Galcheva-Gargova, Z., Karas, R. H., Mendelsohn, M. E., and Shaul, P. W. (1999) J Clin Invest 103, 401-406
Chien, P. Y., Ito, M., Park, Y., Tagami, T., Gehm, B. D., and Jameson, J. L. (1999) Mol. Endocrinol 13, 2122-2136
Clarke, R, Brunner, N., Katzenellenbogen, B. S., Thompson, E. W., Norman, M. J., Koppi, C., Paik, S., Lippman, M. E., and Dickson, R. B. (1989) Proc. Natl Acad. Sci. USA 86, 3649-3653
Clarke, R., Leonessa, F., Welch, J. N., and Skaar, T. C. (2001) PharmacoL Rev. 53, 25-71
Clarke, R., Liu, M. C., Bouker, K. B., Gu, Z., Lee, R. Y., Zhu, Y., Skaar, T. C., Gomez, B.,
O'Brien, K., Wang, Y., and Hilakivi-Clarke, L. A. (2003) Oncogene 22, 7316-7339
Clarke, R., Skaar, T. C., Bouker, K. B., Davis, N., Lee, Y. R., Welch, J. N., and Leonessa, F. (2001) J Steroid Biocheni Mol Biol 76, 71-84.
Cress, W. D., and Triezenberg, S. J. (1991) Science 251, 87-90
Dauvois, S., Danielian, P. S., White, R., and Parker, M. G. (1992) Proc Natl Acad Sci USA 89,4037-4041
Dauvois, S., White, R., and Parker, M. G. (1993) J Cell Sci. 106 (Pt 4), 1377-1388
de Haan G, Chusacultanachai S, Mao C, Katzenellenbogen B S, Shapiro D J (2000) Estrogen receptor-KRAB chimeras are potent ligand-dependent repressors of estrogen-regulated gene expression. J Biol Chem 275:13493-501
deConinck, E. C., McPherson, L. A., and Weigel, R. J. (1995) Mol Cell. BioL 15, 2191-2196
Delage-Mourroux, R., Martini, P. G., Choi, I., Kraichely, D. M., Hoeksema, J., and Katzenellenbogen, B. S. (2000) J. Biol. Chem. 275, 35848-35856
Elliston, J. F., Tsai, S. Y., O'Malley, B. W., and Tsai, M. J. (1990) J Biol Chem 265, 11517-11521
Falette, N. S., Fuqua, S. A., Chanmess, G. C., Cheah, M. S., Greene, G. L., and McGuire, W. L. (1990) Cancer Res. 50, 3974-3978
Fan J D, Wagner B L, McDonnell D P (1996) Identification of the sequences within the human complement 3 promoter required for estrogen responsiveness provides insight into the mechanism of tamoxifen mixed agonist activity. Mol Endocrinol 10:1605-16
Ferguson, A. T., and Davidson, N. E. (1997) Crit. Rev. Oncog. 8, 29-46
Ferguson, A. T., Lapidus, R. G., and Davidson, N. E. (1998) Cancer Treat. Res. 94, 255-278
Ferguson, A. T., Lapidus, R. G., Baylin, S. B., and Davidson, N. E. (1995) Cancer Res. 55, 2279-2283
Font de Mora, J., and Brown, M. (2000) Mol. Cell. Biol. 20, 5041-5047
Foster, J. S., Henley, D. C., Ahaned, S., and Wimalasena, J. (2001) Trends Endocrinol Metab 12, 320-327
Freedman, L. P. (1992) Endocr Rev 13, 129-145
Friedman J R, Fredericks W J, Jensen D E, et al. (1996) KAP-1, a novel corepressor for the hignly conserved KRAB repression domain. Genes Dev 10:2067-78
Fujita, T., Kobayashi, Y., Wada, O., Tateishi, Y., Kitada, L., Yamamoto, Y., Takashima, H., Murayama, A., Yano, T., Baba, T., Kato, S., Kawabe, Y., and Yanagisawa, J. (2003) J Biol. 45 Chem. 278, 26704-26714
Fuqua, S. A., Chamness, G. C., and McGuire, W. L. (1993) J Cell Biochem 51, 135-139
Garcia, M., Derocq, D., Freiss, G., and Rochefort, H. (1992) Proc Natl Acad Sci US A 89, 11538-11542
Gerritsen, M. E., Williams, A. J., Neish, A. S., Moore, S., Shi, Y., and Collins, T. (1997) Proc Natl Acad Sci USA 94, 2927-2932
Glass, C. K. (1994) Endocr Rev 15, 391-407
Goel, S., Mani, S., and Perez-Soler, R. (2002) Curr Oncol Rep 4, 9-19
Graham, J. D., Bain, D. L., Richer, J. K., Jackson, T. A., Tung, L., and Horwitz, K. B. (2000) J Steroid Biochem. Mol BioL 74, 255-259
Graham, J. D., Bain, D. L., Richer, J. K., Jackson, T. A., Tung, L., and Horwitz, K. B. (2000) Steroids 65, 579-584 Green, S., Kumar, V., Knist, A., Walter, P., and Chambon, P. (1986) Cold Spring Harbor Symposia on Quantitative Biology 51, 751-758
Grundker, C., and Emons, G. (2003) Reprod Biol Endocrinol 1, 65
Gruvberger, S., Ringner, M., Chen, Y., Panavally, S., Saal, L. H., Borg, A., Femo, M., Peterson, C., and Meltzer, P. S. (2001) Cancer Res 61, 5979-5984
Gu, Q., Korach, K. S., and Moss, R. L. (1999) Endocrinology 140, 660-666
Gu, Z., Lee, R. Y., Skaar, T. C., Bouker, K. B., Welch, J. N., Lu, J., Liu, A., Zhu, Y., Davis, N., Leonessa, F., Brunner, N., Wang, Y., and Clarke, R. (2002) Cancer Res. 62, 3428-3437
Hall, J. M., Couse, J. F., and Korach, K. S. (2001) J Biol Chem 276, 36869-36872
Hayes, J. J., and Tullius, T. D. (1989) Biochemistry 28, 9521-9527
Herskowitz, I. (1987) Nature 329, 219-222
Howell, A., DeFriend, D. J., Robertson, J. F., Blarney, R. W., Anderson, L., Anderson, E., Sutcliffe, F. A., and Walton, P. (1996) Br. J Cancer 74, 300-308.
Howell, A., Osborne, C. K., Morris, C., and Wakeling, A. E. (2000) Cancer 89, 817-825
Huang, J., Li, X., Yi, P., Hilf, R., Bambara, R. A., and Muyan, M. (2004) Mol. Cell Endocrinol. 218, 65-78
Hudelist, G., Czerwenka, K., Kubista, E., Marton, E., Pischinger, K., and Singer, C. F. (2003) Breast Cancer Res. Treat. 78, 193-204
Ince, B. A., Zhuang, Y., Wrenn, C. K., Shapiro, D. J., and Katzenellenbogen, B. S. (1993) J Biol Chem 268, 14026-14032
Inoue, A., Yoshida, N., Omoto, Y., Oguchi, S., Yamori, T., Kiyama, R., and Hayashi, S. (2002) J Mol Endocrinol 29, 175-192
Inoue, T., Kimura, T., Azuma, C., Inazawa, J., Takemura, M., Kikuchi, T., Kubota, Y., Ogita, K., and Saji, F. (1994) J Biol Chem 269, 32451-32456
Jeng, M. H., Jiang, S. Y., and Jordan, V. C. (1994) Cancer Lett. 82, 123-128
Jeng, M. H., ten Dijke, P., Iwata, K. K., and Jordan, V. C. (1993) Molecular & Cellular Endocrinology 97, 115-123
Jeng, M. H., Yue, W., Eischeid, A., Wang, J. P., and Santen, R. J. (2000) Breast Cancer Res. Treat. 62, 167-175
Jensen, E. V. (1996) Annals of the New York Academy of Sciences 784, 1-17
Jensen, E. V., and Jordan, V. C. (2003) Clin Cancer Res 9, 1980-1989
Jordan, V. C., and Morrow, M. (1999) Endocr Rev 20, 253-278
Kang, J. S., and Kim, J. S. (2000) J Biol Chem 275, 8742-8748
Kasten MM, Ayer DE, Stillman DJ (1996) SIN3 -dependent transcriptional repression by interaction with the Mad1 DNA-binding protein. Mol Cell Biol 16:4215-21
Kato, S., Endoh, H., Masuhiro, Y., Kitamoto, T., Uchiyama, S., Sasaki, H., Masushige, S., Gotoh, Y., Nishida, E., Kawashima, H., and et al. (1995) Science 270, 1491-1494
Kato, S., Kitamoto, T., Masuhiro, Y., and Yanagisawa, J. (1998) Oncology 55 Suppl 1, 5-10
Katzenellenbogen BS (2000) Mechanisms of action and cross-talk between estrogen receptor and Progesterone receptor pathways. J Soc Gynecol Investig 7:S33-7
Kim, J. S., Kim, J., Cepek, K. L., Sharp, P. A., and Pabo, C. O. (1997) Proc Natl Acad Sci USA 94, 3616-3620
Klinge, C. M. (2001) Nucleic Acids Res 29, 2905-2919.
Klinge, C. M., Bodenner, D. L., Desai, D., Niles, R. M., and Traish, A. M. (1997) Nucleic Acids Res 25, 1903-1912
Kozarsky, K. F., and Wilson, J. M. (1993) Curr. Opin. Genet. Dev. 3, 499-503
Kraus W L, Katzenellenbogen BS (1993) Regulation of Progesterone receptor gene expression and growth in the rat uterus: modulation of estrogen actions by Progesterone and sex steroid hormone antagonists. Endocrinology 132:2371-9
Kraus, W. L., McInemey, E. M., and Katzenellenbogen, B. S. (1995) Proc Natl Acad Sci USA 92, 12314-12318
Kuumar, R., Mandal, M., Ratzkin, B. J., Liu, N., and Lipton, A. (1996) J Cell. Biochem. 62, 102-112
Kumar, V., and Chambon, P. (1988) Cell 55,145-156
Kumar, V., Green, S., Stack, G., Berry, M., Jin, J. R., and Chambon, P. (1987) Cell 51, 941-951
Kuntz, M. A., and Shapiro, D. J. (1997) J Biol Chem 272, 27949-279456
Kushner, P. J., Agard, D. A., Greene, G. L., Scanlan, T. S., Shiau, A. K., Uht, R. M., and Webb, P. (2000) J Steroid Biochem Mol Biol 74, 311-317.
Kushner, P. J., Hort, E., Shine, J., Baxter, J. D., and Greene, G. L. (1990) Mol. Endocrinol 4, 1465-1473
Larsen, S. S., Madsen, M. W., Jensen, B. L., and Lykkesfeldt, A. E. (¹997) Int. J Cancer 72,1129-1136 Lavinsky, R. M., Jepsen, K., Heinzel, T., Torchia, J., Mullen, T. M., Schiff, R., Del-Rio, A. L., Ricote, M., Ngo, S., Gemsch, J., Hilsenbeck, S. G., Osbome, C. K., Glass, C. K., Rosenfeld, M. G., and Rose, D. W. (1998) Proc Natl Acad Sci USA 95, 2920-2925
Lazennec, G., Alcom, J. L., and Katzenellenbogen, B. S. (1999) Mol Endocrinol 13, 969-980 Lazennec, G., and Katzenellenbogen, B. S. (1999) Mol Cell Endocrinol 149, 93-105
Lazennec, G., Bresson, D., Lucas, A., Chauveau, C., and Vignon, F. (2001) Endocrinology 142, 4120-4130
Lee, E. J., Jakacka, M., Duan, W. R., Chien, P. Y., Martinson, F., Gehrm, B. D., and Jameson, J. L. (2001) Mol Med. 7, 773-782
Levenson, A. S., and Jordan, V. C. (1994) J Steroid Biochem Mol Biol 51, 229-239
Leygue, E., Dotzlaw, H., Watson, P. H., and Murphy, L. C. (1998) Cancer Res. 58, 3197-3201
Li, L. C., Yeh, C. C., Nojima, D., and Dahiya, R. (2000) Biochem. Biophys. Res. Commun. 275, 682-689 Lim, K., and Chae, C. B. (1989) BioTechniques 7, 576-579
Limonta, P., Montagnani Marelli, M., and Moretti, R. M. (2001) Expert Opin Investig Drugs 10, 709-720 Lin, L., and Hwang, P. L. (1991) Biochimica et Biophysica Acta 1082, 177-184
Liu, P. Q., Rebar, E. J., Zhang, L., Liu, Q., Jamieson, A. C., Liang, Y., Qi, H., Li, P. X.,
Chen, B., Mendel, M. C., Zhong, X., Lee, Y. L., Eisenberg, S. P., Spratt, S. K., Case, C. C., and Wolffe, A. P. (2001) J Biol Chem 276, 11323-11334
Lucas, A., Kremer, E. J., Hernmi, S., Luis, J., Vignon, F., and Lazennec, G. (2003) Biochem. Biophys. Res. Commun. 309, 1011-1016
Luisi, B. F., Schwabe, J. W., and Freedman, L. P. (1994) Vitam Horm 49, 1-47
Ma, Z. Q., Tsai, M. J., and Tsai, S. Y. (1999) J Steroid Biochem Mol Biol 69, 155-163.
Madden, T. A., Barrow, D., McClelland, R. A., Gee, J. M., and Nicholson, R. I. (2000) Eur. J Cancer 36 Suppl 4, S34-35
Mader, S., Chambon, P., and White, J. H. (1993) Nucleic Acids Res 21, 1125-1132
Mangelsdorf, D. J., Thummel, C., Beato, M., Herrlich, P., Schutz, G., Umesono, K., Blumberg, B., Kastner, P., Mark, M., Chambon, P., and et al. (1995) Cell 83, 835-839 Mangelsdorf, D. J., Ong, E. S., Dyck, J. A. and Evans, R. M. (1990) Nuclear receptor that identifies a novel retinoic acid response pathway. Nature 345 (6272), 224-229.
Margolin J F, Friedman J R, Meyer W K, Vissing H, Thiesen H J, Rauscher F J, 3rd (1994) Kruppel-associated boxes are potent transcriptional repression domains. Proc Natl Acad Sci USA 91:4509-13
Mazumdar, A., Wang, R. A., Mishra, S. K., Adam, L., Bagheri-Yarrnand, R., Mandal, M., Vadlamudi, R K., and Kumar, R. (2001) Nat Cell Biol 3, 30-37
McDonnell, D. P. (1999) Trends Endocrinol Metab 10, 301-311
McKenna, N. J., Lanz, R. B., and OMalley, B. W. (1999) Endocr Rev 20, 321-344
Miralles F, Gaudelet C, Cavailles V, Rochefort H, Augereau P (1994) Insensitivity of cathepsin D gene to estradiol in endometrial cells is determined by the sequence of its estrogen responsive element. Biochem Biophy Res Comm 203:711-8
Misrahi, M., Atger, M., d'Auriol, L., Loosfelt, H., Meriel, C., Fridlansky, F., Guiochon-Mantel, A., Galibert, F. and Milgrom, E. (1987) Complete amino acid sequence of the human Progesterone receptor deduced from cloned cDNA. Biochem. Biophys. Res. Commun. 143 (2), 740-748.
Modi, S., and Seidman, A. D. (2002) Curr Oncol Rep 4, 47-55
Moore, P. A., Ruben, S. M., and Rosen, C. A. (1993) Mol Cell Biol 13, 1666-1674.
Moosmann P, Georgiev O, Le Douarin B, Bourquin J P, Schaffner W (1996) Transcriptional repression by RING finger protein TIFI beta that interacts with the KRAB repressor domain of KOX1. Nucleic Acids Res 24:4859-67
Moosmann P, Georgiev 0, Thiesen H J, Hagmann M, Schaffner W (1997) Silencing of RNA polymerases II and rn-dependent transcription by the KRAB protein domain of KOX1, a Kruppel-type zinc finger factor. Biol Chem 378:669-77
Mosselman, S., Polman, J., and Dijkema, R. (1996) FEBS Letters 392, 49-53
Murphy, L. C., Leygue, E., Dotzlaw, H., Douglas, D., Coutts, A., and Watson, P. H. (1997) Ann. Med. 29, 221-234
Muyan, M., Yi, P., Sathya, G., Willmert, L. J., Driscoll, M. D., Hilf, R., and Bambara, R. A. (2001) Mol Cell Endocrinol 182, 249-263.
Nardulli, A. M., Lew, D., Erijman, L., and Shapiro, D. J. (1991J Biol Chem 266,24070-24076
Nawrath, M., Pavlovic, J., and Moelling, K. (2000) Inhibition of human hepatopoietic tumor formation by targeting a repressor Myb-KRAB to DNA. Cancer Gene Ther 7(6), 963-972.
Nicholson, R. I., and Gee, J. M. (2000) Br. J Cancer 82, 501-513
Nicholson, R. I., Gee, J. M., Knowlden, J., McClelland, R., Madden, T. A., Barrow, D., and Hutcheson, I. (2003) Breast Cancer Res. Treat. 80 Suppl 1, S29-34; discussion S35
Nicholson, R. I., Hutcheson, I. R., Harper, M. E., Knowlden, J. M., Barrow, D., McClelland, R. A., Jones, H. E., Wakeling, A. E., and Gee, J. M. (2001) Endocr Relat Cancer 8, 175-182
Nicholson, R. I., McClelland, R. A., Robertson, J. F., and Gee, J. M. (1999) Endocr Relat Cancer 6, 373-387
Nilsson, S., Makela, S., Treuter, E., Tujague, M., Thomsen, J., Andersson, G., Enmark, E., Pettersson, K., Warner, M., and Gustafsson, J. A. (2001) Physiol Rev 81, 1535-1565
Nunez, A. M., Berry, M., Imler, J. L., and Chambon, P. (1989) EMBO J 8, 823-829
Osborne, C. K., Boldt, D. H., and Estrada, P. (1984) Cancer Res 44, 1433-1439
Ottaviano, Y. L., Issa, J. P., Parl, F. F., Smith, H. S., Baylin, S. B., and Davidson, N. E. (1994) Cancer Res. 54, 2552-2555
Ozturk-Winder, F., Renner, M., Klein, D., Muller, M., Salmons, B., and Gunzburg, W. H. (2002) Cancer Gene Ther. 9, 421-431
Pabo, C. O., Peisach, E., and Grant, R. A. (2001) Annu Rev Biochem 70, 313-340
Parker, M. G. (1998) Biochem Soc Symp 63, 45-50
Perou, C. M., Sorlie, T., Eisen, M. B., van de Rijn, M., Jeffrey, S. S., Rees, C. A., Pollack, J. R., Ross, D. T., Johnsen, H., Akslen, L. A., Fluge, O., Pergamenschikov, A., Williams, C., Zhu, S. X., Lonning, P. E., Borresen-Dale, A. L., Brown, P. O., and Botstein, D. (2000) Nature 406, 747-752
Picard, D., Kumar, V., Chambon, P., and Yamamoto, K. R. (1990) Cell Regulation 1, 291-299
Pietras, R. J., Arboleda, J., Reese, D. M., Wongvipat, N., Pegram, M. D., Ramos, L., Gonnan, C. M., Parker, M. G., Sliwkowski, M. X., and Slamon, D. J. (1995) Oncogene 10, 2435-2446
Pike, A. C., Brzozowski, A. M., Hubbard, R. E., Bonn, T., Thorsell, A. G., Engstrom, O., Ljunggren, J., Gustafsson, J. A., and Carlquist, M. (1999) EMBO J 18, 4608-4618
Piva, R., Kumar, L. V., Hanau, S., Maestri, I., Rimondi, A. P., Pansini, S. F., Moflica, G., Chambon, P., and del Senno, L. (1989) Biochemistry International 19, 267-275
Rachez, C., Lemon, B. D., Suldan, Z., Bromleigh, V., Gamble, M., Naar, A. M., Erdjument-Bromage, H., Tempst, P., and Freedman, L. P. (1999) Nature 398, 824-828.
Razandi, M., Pedram, A., and Levin, E. R. (2000) Mol Endocrinol 14, 1434-1447
Regier, J. L., Shen, F., and Triezenberg, S. J. (1993) Proc Natl Acad Sci USA 90, 883-887
Rey, J. M., Pujol, P., Callier, P., Cavailles, V., Freiss, G., Maudelonde, T., and Brouillet, J. P. (2000) J Mol Endocrinol 24, 433-440
Richard, S., and Zingg, H. H. (1990) J Biol Chem 265, 6098-6103
Richard, S., and Zingg, H. H. (1991) J Biol Chem 266, 21428-21433
Robertson, J. F., and Blarney, R. W. (2003) Eur. J Cancer 39, 861-869
Russo, J., Hu, Y. F., Yang, X., and Russo, I. H. (2000) J Natl Cancer Inst Monogr, 17-37
Sadowski, I., Ma, J., Triezenberg, S., and Ptashne, M. (1988) Nature 335, 563-564.
Safe, S. (2001) Vitam Horm 62, 231-252
Santen, R. J. (2002) J Clin. Endocrinol Metab. 87, 3007-3012
Sathya, G., Yi, P., Bhagat, S., Bambara, R. A., Hilf, R., and Muyan, M. (2002) Mol Cell. Endocrinol. 192, 171-185.
Schodin, D. J., Zhuang, Y., Shapiro, D. J., and Katzenellenbogen, B. S. (1995) J Biol Chem 270, 31163-31171
Schwabe, J. W., Chapman, L., Finch, J. T., and Rhodes, D. (1993) Cell 75, 567-578
Shalev, E., and Leung, P. C. (2003) J Obstet Gynaecol Can 25, 98-113
Shang, Y., and Brown, M. (2002) Science 295, 2465-2468
Shang, Y., Hu, X., DiRenzo, J., Lazar, M. A., and Brown, M. (2000) Cell 103, 843-852
Simoncini, T., Hafezi-Moghadam, A., Brazil, D. P., Ley, K., Chin, W. W., and Liao, J. K. (2000) Nature 407, 538-541
Simpson, E. R., and Dowsett, M. (2002) Recent Prog. Horm. Res. 57, 317-338
Slamon, D. J., Godolphin, W., Jones, L. A., Holt, J. A., Wong, S. G., Keith, D. E., Levin, W. J., Stuart, S. G., Udove, J., Ullrich, A., and et al. (1989) Science 244, 707-712
Slamon, D. J., Leyland-Jones, B., Shak, S., Fuchs, H., Paton, V., Bajamonde, A., Fleming, T., Eiermann, W., Wolter, J., Pegram, M., Baselga, J., and Norton, L. (2001) N. Engl. J Med. 344, 783-792
Sliwkowski, M. X., Lofgren, J. A., Lewis, G. D., Hotaling, T. E., Fendly, B. M., and Fox, J. A. (1999) Semin. Oncol. 26, 60-70
Sorlie, T., Perou, C. M., Tibshirani, R., Aas, T., Geisler, S., Johnsen, H., Hastie, T., Eisen, M. B., van de Rijn, M., Jeffrey, S. S., Thorsen, T., Quist, H., Matese, J. C., Brown, P. O., Botstein, D., Eystein Lonning, P., and Borresen-Dale, A. L. (2001) Proc Natl Acad Sci USA 98, 10869-10874
Soulez, M., and Parker, M. G. (2001) J Mol Endocrinol 27, 259-274
Speirs, V., Malone, C., Walton, D. S., Kerin, M. J., and Atkin, S. L. (1999) Cancer Res. 59, 5421-5424
Speirs, V., Parkes, A. T., Kerin, M. J., Walton, D. S., Carleton, P. J., Fox, J. N., and Atkin, S. L. (1999) Cancer Res. 59, 525-528
Strahl, B. D., and Allis, C. D. (2000) Nature 403, 41-45.
Stringer, K. F., Ingles, C. J., and Greenblatt, J. (1990) Nature 345, 783-786
Struhl K (1998) Histone acetylation and transcriptional regulatory mechanisms. Genes & Dev 12:599-606
Sundstrom, S. A., Komm, B. S., Xu, Q., Boundy, V., and Lyttle, C. R. (1990) Endocrinology 126, 1449-1456
Sutherland, R. L., Reddel, R. R., and Green, M. D. (1983) Eur J Cancer Clin Oncol 19, 307-318
Tang, C. K., Perez, C., Grunt, T., Waibel, C., Cho, C., and Lupu, R. (1996) Cancer Res. 56, 3350-3358
Tang, Z., Treilleux, I., and Brown, M. (1997) Mol. Cell. Biol. 17, 1274-1280
Tanimoto, K., Eguchi, H., Yoshida, T., Hajiro-Nakanishi, K., and Hayashi, S. (1999) Nucleic Acids Res. 27, 903-909
Teng, C. T., Liu, Y., Yang, N., Walmer, D., and Panella, T. (1992) Mol Endocrinol 6, 1969-1981
Tbiesen HJ (1990) Multiple genes encoding zinc finger domains are expressed in human T cells. New Biol 2:363-74
Tremblay, A., Tremblay, G. B., Labrie, F., and Giguere, V. (1999) Mol Cell 3, 513-519
Tremblay, G. B., Tremblay, A., Labrie, F., and Giguere, V. (1999) Molecular & Cellular Biology 19, 1919-1927
Triezenberg, S. J., Kingsbury, R. C., and McKnight, S. L. (1988) Genes Dev 2, 718-729.
Tzukerman, M. T., Esty, A., Santiso-Mere, D., Danielian, P., Parker, M. G., Stein, R. B., Pike, J. W., and McDonnell, D. P. (1994) Mol Endocrinol 8, 21-30
Uesugi, M., Nyanguile, O., Lu, H., Levine, A. J., and Verdine, G. L. (1997) Science 277, 1310-1313
Vik, D. P., Amiguet, P., Moffat, G. J., Fey, M., Amiguet-Barras, F., Wetsel, R. A., and Tack, B. F. (1991) Biochemistry 30, 1080-1085
Vladusic, E. A., Homby, A. E., Guerra-Vladusic, F. K., Lakins, J., and Lupu, R. (2000) Oncol Rep 7, 157-167
Vogel, C. L., Cobleigh, M. A., Tripathy, D., Gutheil, J. C., Harris, L. N., Fehrenbacher, L., Slamon, D. J., Murphy, M., Novotny, W. F., Burchmore, M., Shak, S., Stewart, S. J., and Press, M. (2002) J Clin. OncoL 20, 719-726
Wakeling, A. E. (2000) Endocr Relat Cancer 7, 17-28
Wakeling, A. E., Nicholson, R. I., and Gee, J. M. (2001) Clin Cancer Res 7, 4350s-4355s; discussion 4411 s-4412s
Wang, M., Dotzlaw, H., Fuqua, S. A., and Murphy, L. C. (1997) Breast Cancer Res Treat 44, 145-151
Wang, W., Smith, R., 3rd, Burghardt, R., and Safe, S. H. (1997) Mol Cell Endocrinol 133, 49-62
Wang, Y., and Miksicek, R. J. (1991) Mol Endocrinol 5, 1707-1715
Webb, P., Nguyen, P., Shinsako, J., Anderson, C., Feng, W., Nguyen, M. P., Chen, D., Huang, S. M., Subramanian, S., McKinemey, E., Katzenellenbogen, B. S., Stallcup, M. R., and Kushner, P. J. (1998) Mol. Endocrinol. 12, 1605-1618.
Weigel, R. J., and deconinck, E. C. (1993) Cancer Res. 53, 3472-3474
Yang, X., Ferguson, A. T., Nass, S. J., Phillips, D. L., Butash, K. A., Wang, S. M., Herman, J. G., and Davidson, N. E. (2000) Cancer Res. 60, 6890-6894.
Yang, X., Phillips, D. L., Ferguson, A. T., Nelson, W. G., Herman, J. G., and Davidson, N. E. (2001) Cancer Res. 61, 7025-7029.
Yang, X., Yan, L., and.Davidson, N. E. (2001) Endocr Relat Cancer 8, 115-127
Yarden, Y. (2001) Oncology 61 Suppl 2, 1-13
Yi, P., Bhagat, S., Hilf, R., Bambara, R. A., and Muyan, M. (2002) Mol Endocrinol 16, 1810-1827
Yi, P., Driscoll, M. D., Huang, J., Bhagat, S., Hilf, R., Bambara, R. A., and Muyan, M. (2002) Mol Endocrinol 16, 674-693
Young, L. S., and Mautner, V. (2001) Gut 48, 733-736
Zajchowski, D. A., Sager, R., and Webster, L. (1993) Cancer Res 53, 5004-5011

Zhang, L., Spratt, S. K., Liu, Q., Johnstone, B., Qi, H., Raschke, E. E., Jamieson, A. C., Rebar, E. J., Wolffe, A. P., and Case, C. C. (2000) J Biol Chem 275, 33850-33860

| | |
|---|---|
| H. | Sequences |

| | |
|---|---|
| 1. | SEQ ID NO: 1 |

AGAACA

| | |
|---|---|
| 2. | SEQ ID NO: 2 |

AGGTCA

| | |
|---|---|
| 3. | SEQ ID NO: 3 |

AGAACAnnnTGTTCT

| | |
|---|---|
| 4. | SEQ ID NO: 4 |

AGGTCAnAGGTCA

| | |
|---|---|
| 5. | SEQ ID NO: 5 |

AGGTCATGACCT

| | |
|---|---|
| 6. | SEQ ID NO: 6 human pS2 gene ERE |

GGTCAcggTGGCC

| | |
|---|---|
| 7. | SEQ ID NO: 7 human oxytocin gene ERE |

GGTGAcctTGACC

| | |
|---|---|
| 8. | SEQ ID NO: 8 human lactoferrin gene ERE |

GGTCAaggCGATC

| | |
|---|---|
| 9. | SEQ ID NO: 9 |

GGTCAnnnTGACC

| | |
|---|---|
| 10. | SEQ ID NO: 10 human angitensinogen gene ERE |

GGGCAtcgTGACC

| | |
|---|---|
| 11. | SEQ ID NO: 11 human BCL-2 gene ERE |

GGTCGccaGGACC

| | |
|---|---|
| 12. | SEQ ID NO: 12 human BRCA-1 gene ERE |

GGTCAggcTGGTC

| | |
|---|---|
| 13. | SEQ ID NO: 13 human Calbinding-D9 gene ERE |

GGCCGggcTGACC

| | |
|---|---|
| 14. | SEQ ID NO: 14 human cathepsin D gene ERE |

GGCCGggcTGACC

| | |
|---|---|
| 15. | SEQ ID NO: 15 human choline acetyltransferase gene ERE |

GGCCAcgaTGACA

| | |
|---|---|
| 16. | SEQ ID NO: 16 human complement-3 gene ERE |

GGTGGcccTGACC

| | |
|---|---|
| 17. | SEQ ID NO: 17 human cytochorome c oxidase subunit VIIa-related protein |
| | (COX7RP) gene ERE |

GGTCAaggTGACC

| | |
|---|---|
| 18. | SEQ ID NO: 18 human estrogen responsive finger protein gene ERE |

GGTCAtggTGACC

| | |
|---|---|
| 19. | SEQ ID NO: 19 human Progesterone receptor gene ERE |

GCAGGagcTGACC Progesterone

| | |
|---|---|
| 20. | SEQ ID NO: 20 human pS2 gene ERE |

GGTCAgcgTGGCC

| | |
|---|---|
| 21. | SEQ ID NO: 21 human VEGF gene ERE |

AATCAgacTGACT

| | |
|---|---|
| 22. | SEQ ID NO: 22 human Alu gene ERE |

GGTCAggcTGGTC

| | |
|---|---|
| 23. | SEQ ID NO: 23 ERα: (Nucleotide Sequence Accession Number: M12674); DNA |
| | Binding Domain Amino Acid Sequence |

TRYCAVCNDYASGYHYGVWSCEGCKAFFKRSIQGHNDYMCPATNQCTIDKNRRK
SCQACRLRKCYEVGM

| | |
|---|---|
| 24. | SEQ ID NO: 24 ERα Hinge Domain Amino Acid Sequence |

MKGGIRKDRRGGRMLKHKRQRDDGEGRGEVGSAGDMRAANLWPSPLMIKRS

| | |
|---|---|
| 25. | SEQ ID NO: 25 ERβ: (Nucleotide Sequence Accession Number: NM_001437): |
| | DNA Binding Domain Amino Acid Sequence |

DAHFCAVCSDYASGYHYGVWSCEGCKAFFKRSIQGHNDYICPATNQCTIDKNRRK
SGQACRLRKCYEVGM

| | |
|---|---|
| 26. | SEQ ID NO: 26 ERβ Hinge Domain Amino Acid Sequence |

VKCGSRRERCGYRLVRRQRSADEQLHCAG

| | |
|---|---|
| 27. | SEQ ID NO: 27 Glucocorticoid receptor (GR): (Nucleotide Sequence |
| | Accession Number: M10901); DNA Binding Domain Amino Acid Sequence |

PPKLCLVCSDEASGCHYGVLTCGSCKVFFKRAVEGQHNYLCAGRNDCIIDKIRRKN
CPACRYRKCLQAGMNLEA

| | |
|---|---|
| 28. | SEQ ID NO: 28 Hinge Domain Amino Acid Sequence |

RKTKKKIKGIQQATTGVSQETSENPGNKTIVPATLPQLTPT GR

| | |
|---|---|
| 29. | SEQ ID NO: 29 Androgen receptor (AR): (Nucleotide Sequence Accession |
| | Number: L29496): DNA Binding Domain Amino Acid Sequence |

PQKTCLICGDEASGCHYGALTCGSCKVFFKRAAEGKQKYLCASRNDCTIDKFRRKN
CPSCRLRKCYEAGMTL

| | |
|---|---|
| 30. | SEQ ID NO: 30 Domain Amino Acid Sequence |

GARKLKKLGNLKLQEEGEASSTTSPTEETTQKLTVSHIEGYECQPIFLNVLEAI AR
Hinge

| | |
|---|---|
| 31. | SEQ ID NO: 31 Progesterone Receptor (PR): (Nucleotide Sequence |
| | Accession Number: M15716): DNA Binding Domain Amino Acid Sequence |

PQKICLICGDEASGCHYGVLTCGSCKVFFKRAMEGQHNYLCAGRNDCIVDKIRRKN
CPACRLRKCCQAGMVL

| | |
|---|---|
| 32. | SEQ ID NO: 32 PR Hinge Domain Amino Acid Sequence |

GGRKFKKFNKVRVVRALDAVALPQPVGVPNESQALSQRFTFSPGQDIQLIPPL

| | |
|---|---|
| 33. | SEQ ID NO: 33 Thyroid hormone receptor (TR): (Nucleotide Sequence |
| | Accession Number: NM_000461): DNA Binding Domain Amino Acid Sequence |

KDELCVVCGDKATGYHYRCITCEGCKGFFRRTIQKNLHPSYSCKYEGKCVIDKVTR
NQCQECRFKKCIYVGM

| | |
|---|---|
| 34. | SEQ ID NO: 34 TR Hinge Domain Amino Acid Sequence |

ATDLVLDDSKRLAKRKLIEENREKRRREELQKSIGHKPEPTDEEWELIKTVTEAHVA
TNAQGS

| | |
|---|---|
| 35. | SEQ ID NO: 35 Retinoic X Receptor (RXR): (Nucleotide Sequence |
| | Accession Number: NM_002957): DNA Binding Domain Amino Acid Sequence |

TKHICAICGDRSSGKHYGVYSCEGCKGFFKRTVRKDLTYTCRDNKDCLIDKRQRNR
CQYCRYQKCLAMGM

| | |
|---|---|
| 36. | SEQ ID NO: 36 RXR Hinge Domain Amino Acid Sequence |

KREAVQEERQRGKDRNENEVESTSSANEDMPVERILEAELAVEPKTETYVEANMG
LNPS

| | |
|---|---|
| 37. | SEQ ID NO: 37 Retinoic Acid Receptor alpha (RARα): (Nucleotide |
| | Sequence Accession Number: NM_000964): DNA Binding Domain Amino Acid |
| | Sequence |

IYKPCFVCQDKSSGYHYGVSACEGCKGFFRRSIQKNMVYTCHRDKNCIINKVTRNR
CQYCRLQKCFEVGMSK

| | |
|---|---|
| 38. | SEQ ID NO: 38 RARα Hinge Domain Amino Acid Sequence |

ESVRNDRNKKKKEVPKPECSESYTLTPEVGELIEKVRKAHQETFPALCQLGKYTTN
NSSEQRVSLDIDL

| | |
|---|---|
| 39. | SEQ ID NO: 39 Vitamin D receptor (VDR) (Nucleotide Sequence Accession |
| | Number: AF026260): DNA Binding Domain Amino Acid Sequence |

RICGVCGDRATGFHFNAMTCEGCKGFFRRSMKRKALFTCPFNGDCRITKDNRRHC
QACRLKRCVDIGMMK

| | |
|---|---|
| 40. | SEQ ID NO: 40 VDR Hinge Domain Amino Acid Sequence |

EFILTDEEVQRKREMILKRKEEEALKDSLRPKLSEEQQRIIAILLDAHHKTYDPTYSDF
CQFRPP

| | |
|---|---|
| 41. | SEQ ID NO: 41 Human NF-kappa-B transcription factor p65 subunit |
| | (Nucleotide Sequence Accession Number: L19067); Activation Domain |
| | Amino Acid Sequence |

PPQAVAPPAPKPTQAGEGTLSEALLQLQFDDEDLGALLGNSTDPAVFTDLASVDNSE
FQQLLNQGIPVAPHTTEPMLMEYPEAITRLVTGAQRPPDPAPAPLGAPGLPNGLLSG
DEDFSSIADMDFSALLSQIS

| | |
|---|---|
| 42. | SEQ ID NO: 42 Cloning vector pVP16 (Nucleotide Sequence Accession |
| | Number: U89963); VP16 Activation Domain Amino Acid Sequence |

KVAPPTDVSLGDELHLDGEDVAMAHADALDDFDLDMLGDGDSPGPGFTPHDSAPY
GALDMADFEFEQMFTDALGIDEYGGEFPGIRR

| | |
|---|---|
| 43. | SEQ ID NO: 43 Human Mad1 (Nucleotide Sequence Accession Number: |
| | L06895); SID Repression Domain Amino Acid Sequence |

MAAAVRMNIQMLLEAADYLERREREAEHGYASMLP

| | |
|---|---|
| 44. | SEQ ID NO: 44 Human KOX-1 (Nucleotide Sequence Accession Number: |
| | X52332); KRAB Repression Domain Amino Acid Sequence |

MDAKSLTAWSRTLVTFKDVFVDFTREEWKLLDTAQQIVYRNVMLENYKNLVSLG
YQLTKPDVILRLEKGEEPWLVEREIHQETHPDSETA

| | |
|---|---|
| 45. | SEQ ID NO: 45 Estrogen Receptor α (ERα) amino acid sequence |

MTMTLHTKASGMALLHQIQGNELEPLNRPQLKIPLERPLGEVYLDSSKPAVYNYPE
GAAYEFNAAAAANAQVYGQTGLPYGPGSEAAAFGSNGLGGFPPLNSVSPSPLMLL
HPPPQLSPFLQPHGQQVPYYLENEPSGYTVREAGPPAFYRPNSDNRRQGGRERLAST
NDKGSMAMESAKETRYCAVCNDYASGYHYGVWSCEGCKAFFKRSIQGHNDYMC
PATNQCTIDKNRRKSCQACRLRKCYEVGMMKGGIRKDRRGGRMLKHKRQRDDGE
GRGEVGSAGDMRAANLWPSPLMIKRSKKNSLALSLTADQMVSALLDAEPPILYSEY
DPTRPFSEASMMGLLTNLADRELVHMINWAKRVPGFVDLTLHDQVHLLECAWLEI
LMIGLVWRSMEHPVKLLFAPNLLLDRNQGKCVEGMVEIFDMLLATSSRFRMMNLQ
GEEFVCLKSIILNSGVYTFLSSTLKSLEEKDHIHRVLDKITDTLIHLMAKAGLTLQQQ
HQRLAQLLLILSHIRHMSNKGMEHLYSMKCKNVVPLYDLLLEMLDAHRLHAPTSR
GGASVEETDQSHLATAGSTSSHSLQKYYITGEAEGFPATV

| | |
|---|---|
| 46. | SEQ ID NO: 46 ERα nucleic acid sequence |

| | |
|---|---|
| 1 | gaattccaaa attgtgatgt ttcttgtatt tttgatgaag gagaaatact gtaatgatca |
| 61 | ctgtttacac tatgtacact ttaggccagc cctttgtagc gttatacaaa ctgaaagcac |
| 121 | accggacccg caggctcccg gggcagggcc ggggccagag ctcgcgtgtc ggcgggacat |
| 181 | gcgctgcgtc gcctctaacc tcgggctgtg ctctttttcc aggtggcccg ccggtttctg |
| 241 | agccttctgc cctgcgggga cacggtctgc accctgcccg cggccacgga ccatgaccat |
| 301 | gaccctccac accaaagcat ctgggatggc cctactgcat cagatccaag ggaacgagct |
| 361 | ggagcccctg aaccgtccgc agctcaagat ccccctggag cggcccctgg gcgaggtgta |
| 421 | cctggacagc agcaagcccg ccgtgtacaa ctaccccgag ggcgccgcct acgagttcaa |
| 481 | cgccgcggcc gccgccaacg cgcaggtcta cggtcagacc ggcctcccct acggccccgg |
| 541 | gtctgaggct gcggcgttcg gctccaacgg cctggggggt ttccccccac tcaacagcgt |
| 601 | gtctccgagc ccgctgatgc tactgcaccc gccgccgcag ctgtcgcctt tcctgcagcc |
| 661 | ccacggccag caggtgccct actacctgga gaacgagccc agcggctaca cggtgcgcga |
| 721 | ggccggcccg ccggcattct acaggccaaa ttcagataat cgacgccagg gtggcagaga |
| 781 | aagattggcc agtaccaatg acaagggaag tatggctatg gaatctgcca aggagactcg |
| 841 | ctactgtgca gtgtgcaatg actatgcttc aggctaccat tatggagtct ggtcctgtga |
| 901 | gggctgcaag gccttcttca agagaagtat tcaaggacat aacgactata tgtgtccagc |
| 961 | caccaaccag tgcaccattg ataaaaacag gaggaagagc tgccaggcct gccggctccg |
| 1021 | caaatgctac gaagtgggaa tgatgaaagg tgggatacga aaagaccgaa gaggagggag |
| 1081 | aatgttgaaa cacaagcgcc agagagatga tggggagggc aggggtgaag tggggtctgc |
| 1141 | tggagacatg agagctgcca acctttggcc aagcccgctc atgatcaaac gctctaagaa |
| 1201 | gaacagcctg gccttgtccc tgacggccga ccagatggtc agtgccttgt tggatgctga |
| 1261 | gccccccata ctctattccg agtatgatcc taccagaccc ttcagtgaag cttcgatgat |
| 1321 | gggcttactg accaacctgg cagacaggga gctggttcac atgatcaact gggcgaagag |
| 1381 | ggtgccaggc tttgtggatt tgaccctcca tgatcaggtc caccttctag aatgtgcctg |
| 1441 | gctagagatc ctgatgattg gtctcgtctg gcgctccatg gagcacccag tgaagctact |
| 1501 | gtttgctcct aacttgctct tggacaggaa ccagggaaaa tgtgtagagg gcatggtgga |
| 1561 | gatcttcgac atgctgctgg ctacatcatc tcggttccgc atgatgaatc tgcagggaga |
| 1621 | ggagtttgtg tgcctcaaat ctattatttt gcttaattct ggagtgtaca catttctgtc |
| 1681 | cagcaccctg aagtctctgg aagagaagga ccatatccac cgagtcctgg acaagatcac |
| 1741 | agacactttg atccacctga tggccaaggc aggcctgacc ctgcagcagc agcaccagcg |
| 1801 | gctggcccag ctcctcctca tcctctccca catcaggcac atgagtaaca aaggcatgga |
| 1861 | gcatctgtac agcatgaagt gcaagaacgt ggtgcccctc tatgacctgc tgctggagat |
| 1921 | gctggacgcc caccgcctac atgcgcccac tagccgtgga ggggcatccg tggaggagac |
| 1981 | ggaccaaagc cacttggcca ctgcgggctc tacttcatcg cattccttgc aaaagtatta |
| 2041 | catcacgggg gaggcagagg gtttccctgc cacagtctga gagctccctg gc |

| | |
|---|---|
| 47. | SEQ ID NO: 47 ERα DBD nucleic acid sequence |

| | |
|---|---|
| | actcg |
| 841 | ctactgtgca gtgtgcaatg actatgcttc aggctaccat tatggagtct ggtcctgtga |
| 901 | gggctgcaag gccttcttca agagaagtat tcaaggacat aacgactata tgtgtccagc |
| 961 | caccaaccag tgcaccattg ataaaaacag gaggaagagc tgccaggcct gccggctccg |
| 1021 | caaatgctac gaagtgggaa tg |

| | |
|---|---|
| 48. | SEQ ID NO: 48 ERα hinge domain nucleic acid sequence |

| | |
|---|---|
| | atgaaagg tgggatacga aaagaccgaa gaggagggag |
| 1081 | aatgttgaaa cacaagcgcc agagagatga tggggagggc aggggtgaag tggggtctgc |
| 1141 | tggagacatg agagctgcca acctttggcc aagcccgotc atgatcaaac gctct |

| | |
|---|---|
| 49. | SEQ ID NO: 49 Estrogen Receptor β (ERβ) amino acid sequence |

MDIKNSPSSLNSPSSYNCSQSILPLEHGSIYIPSSYVDSHHEYPAMTFYSPAVMNYSIP
SNVTNLEGGPGRQTTSPNVLWPTPGHLSPLVVHRQLSHLYAEPQKSPWCEARSLEH
TLPVNRETLKRKVSGNRCASPVTGPGSKRDAHFCAVCSDYASGYHYGVWSCEGCK
AFFKRSIQGHNDYICPATNQCTIDKNRRKSCQACRLRKCYEVGMVKCGSRRBRCGY
RLVRRQRSADEQLHCAGKAKRSGGHAPRVRELLLDALSPEQLVLTLLEAEPPHVLIS
RPSAPFTEASMMMSLTKLADKELVHMISWAKKIPGFVELSLFDQVRLLESCWMEVL
MMGLMWRSIDHPGKLIFAPDLVLDRDEGKCVEGILEIFDMLLATTSRFRELKLQHKE
YLCVKAMILLNSSMYPLVTATQDADSSRKLAHLLNAVTDALVWVIAKSGISSQQQS
MRLANLLMLLSHVRHASNKGMEHLLNMKCKNVVPVYDLLLEMLNAHVLRGCKSS
ITGSECSPAEDSKSKEGSQNPQSQ

| | |
|---|---|
| 50. | SEQ ID NO: 50 ERB nucleic acid sequence |

| | |
|---|---|
| 1 | tttcagtttc tccagctgct ggctttttgg acacccactc ccccgccagg aggcagttgc |
| 61 | aagcgcggag gctgcgagaa ataactgcct cttgaaactt gcagggcgaa gagcaggcgg |
| 121 | cgagcgctgg gccggggagg gaccacccga gctgcgacgg gctctggggc tgcggggcag |
| 181 | ggctggcgcc cggagcctga gctgcaggag gtgcgctcgc tttcctcaac aggtggcggc |
| 241 | ggggcgcgcg ccgggagacc ccccctaatg cgggaaaagc acgtgtccgc attttagaga |
| 301 | aggcaaggcc ggtgtgttta tctgcaagcc attatacttg cccacgaatc tttgagaaca |
| 361 | ttataatgac ctttgtgcct cttcttgcaa ggtgttttct cagctgttat ctcaagacat |
| 421 | ggatataaaa aactcaccat ctagccttaa ttctccttcc tcctacaact gcagtcaatc |
| 481 | catcttaccc ctggagcacg gctccatata cataccttcc tcctatgtag acagccacca |
| 541 | tgaatatcca gccatgacat tctatagccc tgctgtgatg aattacagca ttcccagcaa |
| 601 | tgtcactaac ttggaaggtg ggcctggtcg gcagaccaca agcccaaatg tgttgtggcc |
| 661 | aacacctggg cacctttctc ctttagtggt ccatcgccag ttatcacatc tgtatgcgga |
| 721 | acctcaaaag agtccctggt gtgaagcaag atcgctagaa cacaccttac ctgtaaacag |
| 781 | agagacactg aaaaggaagg ttagtgggaa ccgttgcgcc agccctgtta ctggtccagg |
| 841 | ttcaaagagg gatgctcact tctgcgctgt ctgcagcgat tacgcatcgg gatatcacta |
| 901 | tggagtctgg tcgtgtgaag gatgtaaggc cttttttaaa agaagcattc aaggacataa |
| 961 | tgattatatt tgtccagcta caaatcagtg tacaatcgat aaaaaccggc gcaagagctg |
| 1021 | ccaggcctgc cgacttcgga agtgttacga agtgggaatg gtgaagtgtg gctcccggag |
| 1081 | agagagatgt gggtaccgcc ttgtgcggag acagagaagt gccgacgagc agctgcactg |
| 1141 | tgccggcaag gccaagagaa gtggcggcca cgcgccccga gtgcgggagc tgctgctgga |
| 1201 | cgccctgagc cccgagcagc tagtgctcac cctcctggag gctgagccgc cccatgtgct |
| 1261 | gatcagccgc cccagtgcgc ccttcaccga ggcctccatg atgatgtccc tgaccaagtt |
| 1321 | ggccgacaag gagttggtac acatgatcag ctgggccaag aagattcccg gctttgtgga |
| 1381 | gctcagcctg ttcgaccaag tgcggctctt ggagagctgt tggatggagg tgttaatgat |
| 1441 | ggggctgatg tggcgctcaa ttgaccaccc cggcaagctc atctttgctc cagatcttgt |
| 1501 | tctggacagg gatgagggga aatgcgtaga aggaattctg gaaatctttg acatgctcct |
| 1561 | ggcaactact tcaaggtttc gagagttaaa actccaacac aaagaatatc tctgtgtcaa |
| 1621 | ggccatgatc ctgctcaatt ccagtatgta ccctctggtc acagcgaccc aggatgctga |
| 1681 | cagcagccgg aagctggctc acttgctgaa cgccgtgacc gatgctttgg tttgggtgat |
| 1741 | tgccaagagc ggcatctcct cccagcagca atccatgcgc ctggctaacc tcctgatgct |
| 1801 | cctgtcccac gtcaggcatg cgagtaacaa gggcatggaa catctgctca acatgaagtg |
| 1861 | caaaaatgtg gtcccagtgt atgacctgct gctggagatg ctgaatgccc acgtgcttcg |
| 1921 | cgggtgcaag tcctccatca cggggtccga gtgcagcccg gcagaggaca gtaaaagcaa |
| 1981 | agagggctcc cagaacccac agtctcagtg a |

| | |
|---|---|
| 51. | SEQ ID NO: 51 ERβ DBD nucleic acid sequence |

| | |
|---|---|
| | gatgctcact tctgcgctgt ctgcagcgat tacgcatcgg gatatcacta |
| 901 | tggagtctgg tcgtgtgaag gatgtaaggc cttttttaaa agaagcattc aaggacataa |
| 961 | tgattatatt tgtccagcta caaatcagtg tacaatcgat aaaaaccggc gcaagagctg |
| 1021 | ccaggcctgc cgacttcgga agtgttacga agtgggaatg |

| | |
|---|---|
| 52. | SEQ ID NO: 52 ERβ hinge domain nucleic acid sequence |

| | |
|---|---|
| | gtgaagtgtg gctcccggag |
| 1081 | agagagatgt gggtaccgcc ttgtgcggag acagagaagt gccgacgagc agctgcactg |
| 1141 | tgccggc |

| | |
|---|---|
| 53. | SEQ ID NO: 53 Glucocorticoid Receptor (GR) amino acid sequence |

MDSKESLTPGREENPSSVLAQERGDVMDFYKTLRGGATVKVSASSPSLAVASQSDS
KQRRLLVDFPKGSVSNAQQPDLSKAVSLSMGLYMGETETKVMGNDLGFPQQGQIS
LSSGETDLKLLEESIANLNRSTSVPENPKSSASTAVSAAPTEKEFPKTHSDVSSEQQH
LKGQTGTNGGNVKLYTTDQSTFDILQDLEFSSGSPGKETNESPWRSDLLIDENCLLSP
LAGEDDSFLLEGNSNEDCKPLILPDTKPKIKDNGDLVLSSPSNVTLPQVKTEKEDFIE
LCTPGVIKQEKLGTVYCQASFPGANIIGNKMSAISVHGVSTSGGQMYHYDMNTASL
SQQQDQKPIFNVIPPIPVGSENWNRCQGSGDDNLTSLGTLNFPGRTVFSNGYSSPSM
RPDVSSPPSSSSTATTGPPPKLCLVCSDEASGCHYGVLTCGSCKVFFKRAVEGQHNY
LCAGRNDCIIDKIRRKNCPACRYRKCLQAGMNLEARKTKKKIKGIQQATTGVSQET
SENPGNKTIVPATLPQLTPTLVSLLEVIEPEVLYAGYDSSVPDSTWRIMTTLNMLGGR
QVIAAVKWAKAIPGFRNLHLDDQMTLLQYSWMFLMAFALGWRSYRQSSANLLCF
APDLIINEQRMTLPCMYDQCKHMLYVSSELHRLQVSYEEYLCMKTLLLLSSVPKDG
LKSQELFDEIRMTYIKELGKAIVKREGNSSQNWQRFYQLTKLLDSMHEVVENLLNY
CFQTFLDKTMSIEFPEMLAEIITNQIPKYSNGNIKKLLFHQK

| | |
|---|---|
| 54. | SEQ ID NO: 54 GR nucleic acid sequence |

| | |
|---|---|
| 1 | tttttagaaa aaaaaaatat atttccctcc tgctccttct gcgttcacaa gctaagttgt |
| 61 | ttatctcggc tgcggcggga actgcggacg gtggcgggcg agcggctcct ctgccagagt |
| 121 | tgatattcac tgatggactc caaagaatca ttaactcctg gtagagaaga aaaccccagc |
| 181 | agtgtgcttg ctcaggagag gggagatgtg atggacttct ataaaaccct aagaggagga |
| 241 | gctactgtga aggtttctgc gtcttcaccc tcactggctg tcgcttctca atcagactcc |
| 301 | aagcagcgaa gacttttggt tgattttcca aaaggctcag taagcaatgc gcagcagcca |
| 361 | gatctgtcca aagcagtttc actctcaatg ggactgtata tgggagagac agaaacaaaa |
| 421 | gtgatgggaa atgacctggg attcccacag cagggccaaa tcagcctttc ctcgggggaa |
| 481 | acagacttaa agcttttgga agaaagcatt gcaaacctca ataggtcgac cagtgttcca |
| 541 | gagaacccca agagttcagc atccactgct gtgtctgctg cccccacaga gaaggagttt |
| 601 | ccaaaaactc actctgatgt atcttcagaa cagcaacatt tgaagggcca gactggcacc |
| 661 | aacggtggca atgtgaaatt gtataccaca gaccaaagca cctttgacat tttgcaggat |
| 721 | ttggagtttt cttctgggtc cccaggtaaa gagacgaatg agagtccttg gagatcagac |
| 781 | ctgttgatag atgaaaactg tttgctttct cctctggcgg gagaagacga ttcattcctt |
| 841 | ttggaaggaa actcgaatga ggactgcaag cctctcattt taccggacac taaacccaaa |
| 901 | attaaggata atggagatct ggttttgtca agccccagta atgtaacact gccccaagtg |
| 961 | aaaacagaaa aagaagattt catcgaactc tgcacccctg gggtaattaa gcaagagaaa |
| 1021 | ctgggcacag tttactgtca ggcaagcttt cctggagcaa atataattgg taataaaatg |
| 1081 | tctgccattt ctgttcatgg tgtgagtacc tctggaggac agatgtacca ctatgacatg |
| 1141 | aatacagcat ccctttctca acagcaggat cagaagccta tttttaatgt cattccacca |
| 1201 | attcccgttg gttccgaaaa ttggaatagg tgccaaggat ctggagatga caacttgact |
| 1261 | tctctgggga ctctgaactt ccctggtcga acagtttttt ctaatggcta ttcaagcccc |
| 1321 | agcatgagac cagatgtaag ctctcctcca tccagctcct caacagcaac aacaggacca |
| 1381 | cctcccaaac tctgcctggt gtgctctgat gaagcttcag gatgtcatta tggagtctta |
| 1441 | acttgtggaa gctgtaaagt tttcttcaaa agagcagtgg aaggacagca caattaccta |
| 1501 | tgtgctggaa ggaatgattg catcatcgat aaaattcgaa gaaaaaactg cccagcatgc |
| 1561 | cgctatcgaa aatgtcttca ggctggaatg aacctggaag ctcgaaaaac aaagaaaaaa |
| 1621 | ataaaaggaa ttcagcaggc cactacagga gtctcacaag aaacctctga aaatcctggt |
| 1681 | aacaaaacaa tagttcctgc aacgttacca caactcaccc ctaccctggt gtcactgttg |
| 1741 | gaggttattg aacctgaagt gttatatgca ggatatgata gctctgttcc agactcaact |
| 1801 | tggaggatca tgactacgct caacatgtta ggagggcggc aagtgattgc agcagtgaaa |
| 1861 | tgggcaaagg caataccagg tttcaggaac ttacacctgg atgaccaaat gaccctactg |
| 1921 | cagtactcct ggatgtttct tatggcattt gctctggggt ggagatcata tagacaatca |
| 1981 | agtgcaaacc tgctgtgttt tgctcctgat ctgattatta atgagcagag aatgactcta |
| 2041 | ccctgcatgt acgaccaatg taaacacatg ctgtatgttt cctctgagtt acacaggctt |
| 2101 | caggtatctt atgaagagta tctctgtatg aaaaccttac tgcttctctc ttcagttcct |
| 2161 | aaggacggtc tgaagagcca agagctattt gatgaaatta gaatgaccta catcaaagag |
| 2221 | ctaggaaaag ccattgtcaa gagggaagga aactccagcc agaactggca gcggttttat |
| 2281 | caactgacaa aactcttgga ttctatgcat gaagtggttg aaaatctcct taactattgc |
| 2341 | ttccaaacat ttttggataa gaccatgagt attgaattcc ccgagatgtt agctgaaatc |
| 2401 | atcaccaatc agataccaaa atattcaaat ggaaatatca aaaaacttct gtttcatcaa |
| 2461 | aagtgactgc cttaataaga atggttgcct taaagaaagt cgaattaata gcttttattg |
| 2521 | tataaactat cagtttgtcc tgtagaggtt ttgttgtttt attttttatt gttttcatct |
| 2581 | gttgttttgt tttaaatacg cactacatgt ggtttataga gggccaagac ttggcaacag |
| 2641 | aagcagttga gtcgtcatca cttttcagtg atgggagagt agatggtgaa atttattagt |
| 2701 | taatatatcc cagaaattag aaaccttaat atgtggacgt aatctccaca gtcaaagaag |
| 2761 | gatggcacct aaaccaccag tgcccaaagt ctgtgtgatg aactttctct tcatactttt |
| 2821 | tttcacagtt ggctggatga aattttctag actttctgtt ggtgtatccc ccccctgtat |
| 2881 | agttaggata gcatttttga tttatgcatg gaaacctgaa aaaaagttta caagtgtata |
| 2941 | tcagaaaagg gaagttgtgc cttttatagc tattactgtc tggttttaac aatttccttt |
| 3001 | atatttagtg aactacgctt gctcattttt tcttacataa ttttttattc aagttattgt |
| 3061 | acagctgttt aagatgggca gctagttcgt agctttccca aataaactct aaacattaat |
| 3121 | caatcatctg tgtgaaaatg ggttggtgct tctaacctga tggcacttag ctatcagaag |
| 3181 | accacaaaaa ttgactcaaa tctccagtat tcttgtcaaa aaaaaaaaaa aaaaagctca |
| 3241 | tattttgtat atatctgctt cagtggagaa ttatataggt tgtgcaaatt aacagtccta |
| 3301 | actggtatag agcacctagt ccagtgacct gctgggtaaa ctgtggatga tggttgcaaa |
| 3361 | agactaattt aaaaaataac taccaagagg ccctgtctgt acctaacgcc ctatttttgc |
| 3421 | aatggctata tggcaagaaa gctggtaaac tatttgtctt tcaggacctt ttgaagtagt |
| 3481 | ttgtataact tcttaaaagt tgtgattcca gataaccagc tgtaacacag ctgagagact |
| 3541 | tttaatcaga caaagtaatt cctctcacta aactttaccc aaaaactaaa tctctaatat |
| 3601 | ggcaaaaatg gctagacacc cattttcaca ttcccatctg tcacoaattg gttaatcttt |
| 3661 | cctgatggta caggaaagct cagctactga tttttgtgat ttagaactgt atgtcagaca |
| 3721 | tccatgtttg taaaactaca catccctaat gtgtgccata gagtttaaca caagtcctgt |
| 3781 | gaatttcttc actgttgaaa attattttaa acaaaataga agctgtagta gccctttctg |
| 3841 | tgtgcacctt accaactttc tgtaaactca aaacttaaca tatttactaa gccacaagaa |
| 3901 | atttgatttc tattcaaggt ggccaaatta tttgtgtaat agaaaactga aaatctaata |
| 3961 | ttaaaaatat ggaacttcta atatattttt atatttagtt atagtttcag atatatatca |
| 4021 | tattggtatt cactaatctg ggaagggaag ggctactgca gctttacatg caatttatta |
| 4081 | aaatgattgt aaaatagctt gtatagtgta aaataagaat gatttttaga tgagattgtt |
| 4141 | ttatcatgac atgttatata ttttttgtag gggtcaaaga aatgctgatg gataacctat |
| 4201 | atgatttata gtttgtacat gcattcatac aggcagcgat ggtctcagaa accaaacagt |
| 4261 | ttgctctagg ggaagaggga gatggagact ggtcctgtgt gcagtgaagg ttgctgaggc |
| 4321 | tctgacccag tgagattaca gaggaagtta tcctctgcct cccattctga ccacccttct |
| 4381 | cattccaaca gtgagtctgt cagcgcaggt ttagtttact caatctcccc ttgcactaaa |
| 4441 | gtatgtaaag tatgtaaaca ggagacagga aggtggtgct tacatcctta aaggcaccat |
| 4501 | ctaatagcgg gttactttca catacagccc tcccccagca gttgaatgac aacagaagct |
| 4561 | tcagaagttt ggcaatagtt tgcatagagg taccagcaat atgtaaatag tgcagaatct |
| 4621 | cataggttgc caataataca ctaattcctt tctatcctac aacaagagtt tatttccaaa |
| 4681 | taaaatgagg acatgttttt gttttctttg aatgcttttt gaatgttatt tgttattttc |
| 4741 | agtattttgg agaaattatt taataaaaaa acaatcattt gctttttg |

| | |
|---|---|
| 55. | SEQ ID NO: 55 GR DBD nucleic acid sequence |

| | |
|---|---|
| | cctcccaaac tctgcctggt gtgctctgat gaagcttcag gatgtcatta tggagtctta |
| 1441 | acttgtggaa gctgtaaagt tttcttcaaa agagcagtgg aaggacagca caattaccta |
| 1501 | tgtgctggaa ggaatgattg catcatcgat aaaattcgaa gaaaaaactg cccagcatgc |
| 1561 | cgctatcgaa aatgtcttca ggctggaatg aacctggaag ct |

| | |
|---|---|
| 56. | SEQ ID NO: 56 GR hinge domain nucleic acid sequence |

| | |
|---|---|
| | cgaaaaac aaagaaaaaa |
| 1621 | ataaaaggaa ttcagcaggc cactacagga gtctcacaag aaacctctga aaatcctggt |
| 1681 | aacaaaacaa tagttcctgc aacgttacca caactcaccc ctacc |

| | |
|---|---|
| 57. | SEQ ID NO: 57 Androgen Receptor (AR) amino acid sequence |

MQLLQQQQQEAVSEGSSSGRAREASGAPTSSKDNYLGGTSTISDNAKELCKAVSVS
MGLGVEALEHLSPGEQLRGDCMYAPLLGVPPAVRPTPCAPLAECKGSLLDDSAGKS
TEDTAEYSPFKGGYTKGLEGESLGCSGSAAAGSSGTLELPSTLSLYKSGALDEAAAY
QSRDYYNFPLALAGPPPPPPPPHPHARIKLENPLDYGSAWAAAAAQCRYGDLASLH
GAGAAGPGSGSPSAAASSSWHTLFTAEEGQLYGPCGGGGGGGGGGGGGGGGGGG
GGGGGGGGEAEAVAPYGYTRPPQGLAGQESDFTAPDVWYPGGMVSRVPYPSPTC
VKSEMGPWMDSYSGPYGDMRLETARDHVLPIDYYFPPQKTCLICGDEASGCHYGA
LTCGSCKVFFKRAAEGKQKYLCASRNDCTIDKFRRKNCPSCRLRKCYEAGMTLGA
RKLKKLGNLKLQEEGEASSTTSPTEETTQKLTVSHIEGYECQPIFLNVLEAIEPGVVC
AGHDNNQPDSFAALLSSLNELGERQLVHVVKWAKALPGFRNLHVDDQMAVIQYS
WMGLMVFAMGWRSFTNVNSRMLYFAPDLVFNEYRMHKSRMYSQCVRMRHLSQE
FGWLQITPQEFLCMKALLLFSIIPVDGLKNQKFFDELRMNYIKELDRIIACKRKNPTS
CSRRFYQLTKLLDSVQPIARELHQFTFDLLIKSHMVSVDFPEMMAEIISVQVPKILSG
KVKPIYFHTQ

| | |
|---|---|
| 58. | SEQ ID NO: 58 AR nucleic acid sequence |

| | |
|---|---|
| 1 | atgcaactcc ttcagcaaca gcagcaggaa gcagtatccg aaggcagcag cagcgggaga |
| 61 | gcgagggagg cctcgggggc tcccacttcc tccaaggaca attacttagg gggcacttcg |
| 121 | accatttctg acaacgccaa ggagttgtgt aaggcagtgt cggtgtccat gggcctgggt |
| 181 | gtggaggcgt tggagcatct gagtccaggg gaacagcttc ggggggattg catgtacgcc |
| 241 | ccacttttgg gagttccacc cgctgtgcgt cccactcctt gtgccccatt ggccgaatgc |
| 301 | aaaggttctc tgctagacga cagcgcaggc aagagcactg aagatactgc tgagtattcc |
| 361 | cctttcaagg gaggttacac caaagggcta gaaggcgaga gcctaggctg ctctggcagc |
| 421 | gctgcagcag ggagctccgg gacacttgaa ctgccgtcta ccctgtctct ctacaagtcc |
| 481 | ggagcactgg acgaggcagc tgcgtaccag agtcgcgact actacaactt tccactggct |
| 541 | ctggccggac cgccgccccc tccgccgcct ccccatcccc acgctcgcat caagctggag |
| 601 | aacccgctgg actacggcag cgcctgggcg gctgcggcgg cgcagtgccg ctatggggac |
| 661 | ctggcgagcc tgcatggcgc gggtgcagcg ggacccggtt ctgggtcacc ctcagccgcc |
| 721 | gcttcctcat cctggcacac tctcttcaca gccgaagaag gccagttgta tggaccgtgt |
| 781 | ggtggtggtg ggggtggtgg tggcggcggc ggcggcggcg gcggcggcgg cggcggcggc |
| 841 | ggcggcggcg gcggcggcgg cgaggcggaa gctgtagccc cctacggcta cactcggccc |
| 901 | cctcaggggc tggcgggcca ggaaagcgac ttcaccgcac ctgatgtgtg gtaccctggc |
| 961 | ggcatggtga gcagagtgcc ctatcccagt cccacttgtg tcaaaagcga aatgggcccc |
| 1021 | tggatggata gctactccgg accttacggg gacatgcgtt tggagactgc cagggaccat |
| 1081 | gttttgccca ttgactatta ctttccaccc cagaagacct gcctgatctg tggagatgaa |
| 1141 | gcttctgggt gtcactatgg agctctcaca tgtggaagct gcaaggtctt cttcaaaaga |
| 1201 | gccgctgaag ggaaacagaa gtacctgtgc gccagcagaa atgattgcac tattgataaa |
| 1261 | ttccgaagga aaaattgtcc atcttgtcgt cttcggaaat gttatgaagc agggatgact |
| 1321 | ctgggagccc ggaagctgaa gaaacttggt aatctgaaac tacaggagga aggagaggct |
| 1381 | tccagcacca ccagccccac tgaggagaca acccagaagc tgacagtgtc acacattgaa |
| 1441 | ggctatgaat gtcagcccat ctttctgaat gtcctggaag ccattgagcc aggtgtagtg |
| 1501 | tgtgctggac acgacaacaa ccagcccgac tcctttgcag ccttgctctc tagcctcaat |
| 1561 | gaactgggag agagacagct tgtacacgtg gtcaagtggg ccaaggcctt gcctggcttc |
| 1621 | cgcaacttac acgtggacga ccagatggct gtcattcagt actcctggat ggggctcatg |
| 1681 | gtgtttgcca tgggctggcg atccttcacc aatgtcaact ccaggatgct ctacttcgcc |
| 1741 | cctgatctgg ttttcaatga gtaccgcatg cacaagtccc ggatgtacag ccagtgtgtc |
| 1801 | cgaatgaggc acctctctca agagtttgga tggctccaaa tcacccccca ggaattcctg |
| 1861 | tgcatgaaag cactgctact cttcagcatt attccagtgg atgggctgaa aaatcaaaaa |
| 1921 | ttctttgatg aacttcgaat gaactacatc aaggaactcg atcgtatcat tgcatgcaaa |
| 1981 | agaaaaaatc ccacatcctg ctcaagacgc ttctaccagc tcaccaagct cctggactcc |
| 2041 | gtgcagccta ttgcgagaga gctgcatcag ttcacttttg acctgctaat caagtcacac |
| 2101 | atggtgagcg tggactttcc ggaaatgatg gcagagatca tctctgtgca agtgcccaag |
| 2161 | atcctttctg ggaaagtcaa gcccatctat ttccacaccc agtga |

| | |
|---|---|
| 59. | SEQ ID NO: 59 AR DBD nucleic acid sequence |

| | |
|---|---|
| | ccc cagaagacct gcctgatctg tggagatgaa |
| 1141 | gcttctgggt gtcactatgg agctctcaca tgtggaagct gcaaggtctt cttcaaaaga |
| 1201 | gccgctgaag ggaaacagaa gtacctgtgc gccagcagaa atgattgcac tattgataaa |
| 1261 | ttccgaagga aaaattgtcc atcttgtcgt cttcggaaat gttatgaagc agggatgact |
| 1321 | ctg |

| | |
|---|---|
| 60. | SEQ ID NO: 60 AR hinge domain nucleic acid sequence |

| | |
|---|---|
| | ggagccc ggaagctgaa gaaacttggt aatctgaaac tacaggagga aggagaggct |
| 1381 | tccagcacca ccagccccac tgaggagaca acccagaagc tgacagtgtc acacattgaa |
| 1441 | ggctatgaat gtcagcccat ctttctgaat gtcctggaag ccatt |

| | |
|---|---|
| 61. | SEQ ID NO: 61 Progesterone Receptor (PR) amino acid sequence |

MTELKAKGPRAPHVAGGPPSPEVGSPLLCRPAAGPFPGSQTSDTLPEVSAIPISLDGL
LFPRPCQGQDPSDEKTQDQQSLSDVEGAYSRAEATRGAGGSSSSPPEKDSGLLDSVL
DTLLAPSGPGQSQPSPPACEVTSSWCLFGPELPEDPPAAPATQRVLSPLMSRSGCKV
GDSSGTAAAHKVLPRGLSPARQLLLPASESPHWSGAPVKPSPQAAAVEVEEEDGSE
SEESAGPLLKGKPRALGGAAAGGGAAAVPPGAAAGGVALVPKEDSRFSAPRVALV
EQDAPMAPGRSPLATTVMDFIHVPILPLNHALLAARTRQLLEDESYDGGAGAASAF
APPRSSPCASSTPVAVGDFPDCAYPPDAEPKDDAYPLYSDFQPPALKIKEEEEGAEA
SARSPRSYLVAGANPAAFPDFPLGPPPPLPPRATPSRPGEAAVTAAPASASVSSASSS
GSTLECILYKAEGAPPQQGPFAPPPGKAPGASGCLLPRDGLPSTSASAAAAGAAPAL
YPALGLNGLPQLGYQAAVLKEGLPQVYPPYLNYLRPDSEASQSPQYSFESLPQKICLI
CGDEASGCHYGVLTCGSCKVFFKRAMEGQHNYLCAGRNDCIVDKIRRKNCPACRL
RKCCQAGMVLGGRKFKKFNKVRVVRALDAVALPQPVGVPNESQALSQRFTFSPGQ
DIQLIPPLINLLMSIEPDVIYAGHDNTKPDTSSSLLTSLNQLGERQLLSVVKWSKSLPG
FRMLHIDDQITLIQYSWMSLMVFGLGWRSYKHVSGQMLYFAPDLILNEQRMKESSF
YSLCLTMWQIPQEFVKLQVSQEEFLCMKVLLLLNTIPLEGLRSQTQFEEMRSSYIREL
IKAIGLRQKGVVSSSQRFYQLTKLLDNLHDLVKQLHLYCLNTFIQSRALSVEFPEMM
SEVIAAQLPKILAGMVKPLLFHKK

| | |
|---|---|
| 62. | SEQ ID NO: 62 PR nucleic acid sequence |

| | |
|---|---|
| 1 | ctgaccagcg ccgccctccc ccgcccccga cccaggaggt ggagatccct ccggtccagc |
| 61 | cacattcaac acccactttc tcctccctct gcccctatat tcccgaaacc ccctcctcct |
| 121 | tcccttttcc ctcctccctg gagacggggg aggagaaaag gggagtccag tcgtcatgac |
| 181 | tgagctgaag gcaaagggtc cccgggctcc ccacgtggcg ggcggcccgc cctcccccga |
| 241 | ggtcggatcc ccactgctgt gtcgcccagc cgcaggtccg ttcccgggga gccagacctc |
| 301 | ggacaccttg cctgaagttt cggccatacc tatctccctg gacgggctac tcttccctcg |
| 361 | gccctgccag ggacaggacc cctccgacga aaagacgcag gaccagcagt cgctgtcgga |
| 421 | cgtggagggc gcatattcca gagctgaagc tacaaggggt gctggaggca gcagttctag |
| 481 | tcccccagaa aaggacagcg gactgctgga cagtgtcttg gacactctgt tggcgccctc |
| 541 | aggtcccggg cagagccaac ccagccctcc cgcctgcgag gtcaccagct cttggtgcct |
| 601 | gtttggcccc gaacttcccg aagatccacc ggctgccccc gccacccagc gggtgttgtc |
| 661 | cccgctcatg agccggtccg ggtgcaaggt tggagacagc tccgggacgg cagctgccca |
| 721 | taaagtgctg ccccggggcc tgtcaccagc ccggcagctg ctgctcccgg cctctgagag |
| 781 | ccctcactgg tccggggccc cagtgaagcc gtctccgcag gccgctgcgg tggaggttga |
| 841 | ggaggaggat ggctctgagt ccgaggagtc tgcgggtccg cttctgaagg gcaaacctcg |
| 901 | ggctctgggt ggcgcggcgg ctggaggagg agccgcggct gtcccgccgg gggcggcagc |
| 961 | aggaggcgtc gccctggtcc ccaaggaaga ttcccgcttc tcagcgccca gggtcgccct |
| 1021 | ggtggagcag gacgcgccga tggcgcccgg gcgctccccg ctggccacca cggtgatgga |
| 1081 | tttcatccac gtgcctatcc tgcctctcaa tcacgcctta ttggcagccc gcactcggca |
| 1141 | gctgctggaa gacgaaagtt acgacggcgg ggccggggct gccagcgcct ttgccccgcc |
| 1201 | gcggagttca ccctgtgcct cgtccacccc ggtcgctgta ggcgacttcc ccgactgcgc |
| 1261 | gtacccgccc gacgccgagc ccaaggacga cgcgtaccct ctctatagcg acttccagcc |
| 1321 | gcccgctcta aagataaagg aggaggagga aggcgcggag gcctccgcgc gctccccgcg |
| 1381 | ttcctacctt gtggccggtg ccaaccccgc agccttcccg gatttcccgt tggggccacc |
| 1441 | gcccccgctg ccgccgcgag cgaccccatc cagacccggg gaagcggcgg tgacggccgc |
| 1501 | acccgccagt gcctcagtct cgtctgcgtc ctcctcgggg tcgaccctgg agtgcatcct |
| 1561 | gtacaaagcg gagggcgcgc cgccccagca gggcccgttc gcgccgccgc cctgcaaggc |
| 1621 | gccgggcgcg agcggctgcc tgctcccgcg ggacggcctg ccctccacct ccgcctctgc |
| 1681 | cgccgccgcc ggggcggccc ccgcgctcta ccctgcactc ggcctcaacg ggctcccgca |
| 1741 | gctcggctac caggccgccg tgctcaagga gggcctgccg caggtctacc cgccctatct |
| 1801 | caactacctg aggccggatt cagaagccag ccagagccca caatacagct tcgagtcatt |
| 1861 | acctcagaag atttgtttaa tctgtgggga tgaagcatca ggctgtcatt atggtgtcct |
| 1921 | tacctgtggg agctgtaagg tcttctttaa gagggcaatg gaagggcagc acaactactt |
| 1981 | atgtgctgga agaaatgact gcatcgttga taaaatccgc agaaaaaact gcccagcatg |
| 2041 | tcgccttaga aagtgctgtc aggctggcat ggtccttgga ggtcgaaaat ttaaaaagtt |
| 2101 | caataaagtc agagttgtga gagcactgga tgctgttgct ctcccacagc cagtgggcgt |
| 2161 | tccaaatgaa agccaagccc taagccagag attcactttt tcaccaggtc aagacataca |
| 2221 | gttgattcca ccactgatca acctgttaat gagcattgaa ccagatgtga tctatgcagg |
| 2281 | acatgacaac acaaaacctg acacctccag ttctttgctg acaagtctta atcaactagg |
| 2341 | cgagaggcaa cttctttcag tagtcaagtg gtctaaatca ttgccaggtt ttcgaaactt |
| 2401 | acatattgat gaccagataa ctctcattca gtattcttgg atgagcttaa tggtgtttgg |
| 2461 | tctaggatgg agatcctaca aacacgtcag tgggcagatg ctgtattttg cacctgatct |
| 2521 | aatactaaat gaacagcgga tgaaagaatc atcattctat tcattatgcc ttaccatgtg |
| 2581 | gcagatccca caggagtttg tcaagcttca agttagccaa gaagagttcc tctgtatgaa |
| 2641 | agtattgtta cttcttaata caattccttt ggaagggcta cgaagtcaaa cccagtttga |
| 2701 | ggagatgagg tcaagctaca ttagagagct catcaaggca attggtttga ggcaaaaagg |
| 2761 | agttgtgtcg agctcacagc gtttctatca acttacaaaa cttcttgata acttgcatga |
| 2821 | tcttgtcaaa caacttcatc tgtactgctt gaatacattt atccagtccc gggcactgag |
| 2881 | tgttgaattt ccagaaatga tgtctgaagt tattgctgca caattaccca agatattggc |
| 2941 | agggatggtg aaaccccttc tctttcataa aaagtgaatg tcatcttttt cttttaaaga |
| 3001 | attaaatttt gtgg |

| | |
|---|---|
| 63. | SEQ ID NO: 63 PR DBD nucleic acid sequence |

| | |
|---|---|
| | cctcagaag atttgtttaa tctgtgggga tgaagcatca ggctgtcatt atggtgtcct |
| 1921 | tacctgtggg agctgtaagg tcttctttaa gagggcaatg gaagggcagc acaactactt |
| 1981 | atgtgctgga agaaatgact gcatcgttga taaaatccgc agaaaaaact gcccagcatg |
| 2041 | tcgccttaga aagtgctgtc aggctggcat ggtcctt |

| | |
|---|---|
| 64. | SEQ ID NO: 64 PR hinge domain nucleic acid sequence |

| | |
|---|---|
| | gga ggtcgaaaat ttaaaaagtt |
| 2101 | caataaagtc agagttgtga gagcactgga tgctgttgct ctcccacagc cagtgggcgt |
| 2161 | tccaaatgaa agccaagccc taagccagag attcactttt tcaccaggtc aagacataca |
| 2221 | gttgattcca ccactga |

| | |
|---|---|
| 65. | SEQ ID NO: 65 Thyroid hormone Receptor (TR) amino acid sequence |

MTENGLTAWDKPKHCPDREHDWKLVGMSEACLHRKSHSERRSTLKNEQSSPHLIQ
TTWTSSIFHLDHDDVNDQSVSSAQTFQTEEKKCKGYIPSYLDKDELCVVCGDKATG
YHYRCITCEGCKGFFRRTIQKNLHPSYSCKYEGKCVIDKVTRNQCQECRFKKCIYVG
MATDLVLDDSKRLAKRKLIEENREKRRREELQKSIGHKPEPTDEEWELIKTVTEAHV
ATNAQGSHWKQKPKFLPEDIGQAPIVNAPEGGKVDLEAFSHFTKIITPAITRVVDFA
KKLPMFCELPCEDQIILLKGCCMEIMSLRAAVRYDPESETLTLNGEMAVIRGQLKNG
GLGVVSDAIFDLGMSLSSFNLDDTEVALLQAVLLMSSDRPGLACVERIEKYQDSFLL
AFEHYINYRKHHVTHFWPKLLMKVTDLRMIGACHASRFLHMKVECPTELLPPLFEV
FED

| | |
|---|---|
| 66. | SEQ ID NO: 66 TR nucleic acid sequence |

| | |
|---|---|
| 1 | cggcggggat caactttgca tgaataatgt gagtgcgctt ggaaaagaga cctcctgctc |
| 61 | cgcgggctcg gggcaagagc ccgcaggcta ccttccccgg gcaggggcgc tcaacccaac |
| 121 | cggctccagg gcactggtaa tttggctaga ggaccgcgcg gaggcagcgg gatctgcgat |
| 181 | ttccttctgg ttggctgtcc tgcgtgggtg ccaagttcca cacatgattt aatgaataag |
| 241 | aaggagatgt cagtgaaaaa agggatccag aatgattact aacctataac ccccaacagt |
| 301 | atgacagaaa atggccttac agcttgggac aaaccgaagc actgtccaga ccgagaacac |
| 361 | gactggaagc tagtaggaat gtctgaagcc tgcctacata ggaagagcca ttcagagagg |
| 421 | cgcagcacgt tgaaaaatga acagtcgtcg ccacatctca tccagaccac ttggactagc |
| 481 | tcaatattcc atctggacca tgatgatgtg aacgaccaga gtgtctcaag tgcccagacc |
| 541 | ttccaaacgg aggagaagaa atgtaaaggg tacatcccca gttacttaga caaggacgag |
| 601 | ctctgtgtag tgtgtggtga caaagccacc gggtatcact accgctgtat cacgtgtgaa |
| 661 | ggctgcaagg gtttctttag aagaaccatt cagaaaaatc tccatccatc ctattcctgt |
| 721 | aaatatgaag gaaaatgtgt catagacaaa gtcacgcgaa atcagtgcca ggaatgtcgc |
| 781 | tttaagaaat gcatctatgt tggcatggca acagatttgg tgctggatga cagcaagagg |
| 841 | ctggccaaga ggaagctgat agaggagaac cgggagaaaa gacggcggga agagctgcag |
| 901 | aagtccatcg ggcacaagcc agagcccaca gacgaggaat gggagctcat caaaactgtc |
| 961 | accgaagccc atgtggcgac caacgcccaa ggcagccact ggaagcaaaa accgaaattt |
| 1021 | ctgccagaag acattggaca agcaccaata gtcaatgccc cagaaggtgg aaaggttgac |
| 1081 | ttggaagcct tcagccattt tacaaaaatc atcacaccag caattaccag agtggtggat |
| 1141 | tttgccaaaa agttgcctat gttttgtgag ctgccatgtg aagaccagat catcctcctc |
| 1201 | aaaggctgct gcatggagat catgtccctt cgcgctgctg tgcgctatga cccggaaagt |
| 1261 | gagactttaa ccttgaatgg ggaaatggca gtgatacggg gccagctgaa aaatgggggt |
| 1321 | cttggggtgg tgtcagacgc catctttgac ctaggcatgt ctctgtcttc tttcaacctg |
| 1381 | gatgacactg aagtagccct ccttcaggcc gtcctgctga tgtcttcaga tcgcccgggg |
| 1441 | cttgcctgtg ttgagagaat agaaaagtac caagatagtt tcctgctggc ctttgaacac |
| 1501 | tatatcaatt accgaaaaca ccacgtgaca cacttttggc caaaactcct gatgaaggtg |
| 1561 | acagatctgc ggatgatagg agcctgccat gccagccgct tcctgcacat gaaggtggaa |
| 1621 | tgccccacag aactcctccc ccctttgttc ctggaagtgt tcgaggatta gactgactgg |
| 1681 | attccttcct ataattcc |

| | |
|---|---|
| 67. | SEQ ID NO: 67 TR DBD nucleic acid sequence |

| | |
|---|---|
| | aaggacgag |
| 601 | ctctgtgtag tgtgtggtga caaagccacc gggtatcact accgctgtat cacgtgtgaa |
| 661 | ggctgcaagg gtttctttag aagaaccatt cagaaaaatc tccatccatc ctattcctgt |
| 721 | aaatatgaag gaaaatgtgt catagacaaa gtcacgcgaa atcagtgcca ggaatgtcgc |
| 781 | tttaagaaat gcatctatgt tggcatg |

| | |
|---|---|
| 68. | SEQ ID NO: 68 TR hinge domain nucleic acid sequence |

| | |
|---|---|
| | gca acagatttgg tgctggatga cagcaagagg |
| 841 | ctggccaaga ggaagctgat agaggagaac cgggagaaaa gacggcggga agagctgcag |
| 901 | aagtccatcg ggcacaagcc agagcccaca gacgaggaat gggagctcat caaaactgtc |
| 961 | accgaagccc atgtggcgac caacgcccaa ggcagc |

| | |
|---|---|
| 69. | SEQ ID NO: 69 Retinoic X Receptor α (RXRα) amino acid sequence |

MDTKHFLPLDFSTQVNSSLTSPTGRGSMAAPSLHPSLGPGIGSPGQLHSPISTLSSPIN
GMGPPFSVISSPMGPHSMSVPTTPTLGFSTGSPQLSSPMNPVSSSEDIKPPLGLNGVL
KVPAHPSGNMASFTKHICAICGDRSSGKHYGVYSCEGCKGFFKRTVRKDLTYTCRD
NKDCLIDKRQRNRCQYCRYQKCLAMGMKREAVQEERQRGKDRNENEVESTSSAN
EDMPVERILEAELAVEPKTEIYVEANMGLNPSSPNDPVTNICQAADKQLFTLVEWA
KRIPHFSELPLDDQVILLRAGWNELLIASFSHRSIAVKDGILLATGLHVHRNSAHSAG
VGAIFDRVLTELVSKMRDMQMDKTELGCLRAIVLFNPDSKGLSNPAEVEALREKVY
ASLEAYCKHKYPEQPGRFAKLLLRLPALRSIGLKCLEHLFFFKLIGDTPIDTFLMEML
EAPHQMT

| | |
|---|---|
| 70. | SEQ ID NO: 70 RXRα amino acid sequence |

| | |
|---|---|
| 1 | gcgccggggg ccgccgcgcc cgccgcccgc tgcctgcgcc gccggccggg catgagttag |
| 61 | tcgcagacat ggacaccaaa catttcctgc cgctcgattt ctccacccag gtgaactcct |
| 121 | ccctcacctc cccgacgggg cgaggctcca tggctgcccc ctcgctgcac ccgtccctgg |
| 181 | ggcctggcat cggctccccg ggacagctgc attctcccat cagcaccctg agctccccca |
| 241 | tcaacggcat gggcccgcct ttctcggtca tcagctcccc catgggcccc cactccatgt |
| 301 | cggtgcccac cacacccacc ctgggcttca gcactggcag cccccagctc agctcaccta |
| 361 | tgaaccccgt cagcagcagc gaggacatca agccccccct gggcctcaat ggcgtcctca |
| 421 | aggtccccgc ccacccctca ggaaacatgg cttccttcac caagcacatc tgcgccatct |
| 481 | gcggggaccg ctcctcaggc aagcactatg gagtgtacag ctgcgagggg tgcaagggct |
| 541 | tcttcaagcg gacggtgcgc aaggacctga cctacacctg ccgcgacaac aaggactgcc |
| 601 | tgattgacaa gcggcagcgg aaccggtgcc agtactgccg ctaccagaag tgcctggcca |
| 661 | tgggcatgaa gcgggaagcc gtgcaggagg agcggcagcg tggcaaggac cggaacgaga |
| 721 | atgaggtgga gtcgaccagc agcgccaacg aggacatgcc ggtggagagg atcctggagg |
| 781 | ctgagctggc cgtggagccc aagaccgaga cctacgtgga ggcaaacatg gggctgaacc |
| 841 | ccagctcgcc gaacgaccct gtcaccaaca tttgccaagc agccgacaaa cagcttttca |
| 901 | ccctggtgga gtgggccaag cggatcccac acttctcaga gctgcccctg gacgaccagg |
| 961 | tcatcctgct gcgggcaggc tggaatgagc tgctcatcgc ctccttctcc caccgctcca |
| 1021 | tcgccgtgaa ggacgggatc ctcctggcca ccgggctgca cgtccaccgg aacagcgccc |
| 1081 | acagcgcagg ggtgggcgcc atctttgaca gggtgctgac ggagcttgtg tccaagatgc |
| 1141 | gggacatgca gatggacaag acggagctgg gctgcctgcg cgccatcgtc ctctttaacc |
| 1201 | ctgactccaa ggggctctcg aacccggccg aggtggaggc gctgagggag aaggtctatg |
| 1261 | cgtccttgga ggcctactgc aagcacaagt acccagagca gccgggaagg ttcgctaagc |
| 1321 | tcttgctccg cctgccggct ctgcgctcca tcgggctcaa atgcctggaa catctcttct |
| 1381 | tcttcaagct catcggggac acacccattg acaccttcct tatggagatg ctggaggcgc |
| 1441 | cgcaccaaat gacttaggcc tgcgggcoca tcctttgtgc ccacccgttc tggccaccct |
| 1501 | gcctggacgc cagctgttct tctcagcctg agccctgtcc ctgcccttct ctgcctggcc |
| 1561 | tgtttggact ttggggcaca gcctgtcact gctctgccta agagatgtgt tgtcaccctc |
| 1621 | cttatttctg ttactacttg tctgtggccc agggcagtgg ctttcctgag gcagcagcct |
| 1681 | tcgtggcaag aactagcgtg agcccagcca ggcgcctccc caccgggctc tcaggacacc |
| 1741 | ctgccacacc ccacggggct tgggcgacta cagggtcttc gggccccagc cctggagctg |
| 1801 | caggagttgg gaacggggct tttgtttccg ttgctgttta tcgatgctgg ttttcagaat |
| 1861 | tcctgtgtgg ccctcctgtc tggagtgaca tcttcatctg ctctgaatac tggtgcccag |
| 1921 | ccagcccgtg acagcttccc cctaatcagg aggggacagc tgggggcgca agctggtgtg |
| 1981 | tcatcagcaa agacctcagc cgcctcgggg atgagagggg actcgtgggg caagcaagct |
| 2041 | gccctgtgct ctgagtgagg gggaaggtag cccctttttc caaagataac tcacagtttt |
| 2101 | gccctcgagc caatgagaac atgagctgcc ctctgtgcaa ggtttcgggg ccacctccag |
| 2161 | gctgcagggg cgggtcactc acccccctgt tttctctctg ccttggtgtt ctggtttcag |
| 2221 | actcccgact ccccgttcag accagagtgc cccggcccct ccccagcctg agtcttctcc |
| 2281 | ttgctctgcg gggtgggctg aggcttgtce ttgtttcctg cagggctggc cctggctcgg |
| 2341 | gcagggtggg gcatcaccac ctcactggcc ttgctggagg cacagggctc tgcggacctg |
| 2401 | cagccatctg tgaggcccgc ggggatggga ggggaggagg gtggcctgtt ggtttccctc |
| 2461 | agagggggca ggtggcctgg agagagaggg gctcaggaac tgggagcctc gtgggtgggg |
| 2521 | cagatgctcc gcggcctgga gtggctctgc cggggcattg gtgggacccc tgctcaggcc |
| 2581 | ttctctctgg ctgccagttg tgtctaaaag actcttggaa tctgagaacc cggagtcgca |
| 2641 | gcgccctcgg gcctgggcca cacgcaggcc ctggtgggac cacccagcct ggtattgtcc |
| 2701 | acggacagcg ttgttcaccc agagccttac ttgggagcct cactgaacgc ctgctctggt |
| 2761 | tgaaggtggg gtgggggcgg ggcttggggc ctccctggct cagcccagtg cggcctggcg |
| 2821 | ctcctcccgc aggctctgcc cccgggctcc ggtggtgcgg ggccctctca ggttgaactc |
| 2881 | gcctcttttg cactggaagg ccctcccttt ggcctgagta cttttcccgt tcacgcctca |
| 2941 | gtcccgtgga cccagccttt gtcagtggca ggtgcctgaa cagagggtgg atggggggga |
| 3001 | taccggaggg ggtcttgtct tcccagccgc agtctaggaa tgatgcgggg gggtggacgc |
| 3061 | cttctccata gtctttcccc acctggagca ggggcttcct cagtggtgag gggagctgcc |
| 3121 | tacaggttgg accgggaggc agtggcttgg agaggcagct ttccagcctt ggtggggaag |
| 3181 | aaagtgtcca ttctttgcct tcctggagct cccagccaga gctgagctta ggcacccgag |
| 3241 | tggagcctgc agctgagtct gtgcccgaga caggctgtca gagattccag aagcctctcc |
| 3301 | tccccgccgc cctccacccc tgcctttcag cgttgtggat ccctagaggt ggccccctgc |
| 3361 | ccgatccacc gtcctgaggc agagtgttga gcctcatacc tgtaccaggt ccccggccag |
| 3421 | ctgggcccct cccaggcact gccaggaagc cccagctgcc cctggcgggt gtggtggaaa |
| 3481 | tggcaggagg gtgcaggtac tcttggggcc ccagcggtgg gagtgcaaaa gacccaacgc |
| 3541 | caacacctgg tgccttttgc agccagcgcc cacccatccg tgcccggacc cttgggaatg |
| 3601 | cccgcggctc cagaggaaaa agcccaggga cggggcctcc gttgcggggg gtcggctgct |
| 3661 | tcttgggaac tttgtcgttt ccggcgctgg ctggctggct ggctgtaaag cactgaagcc |
| 3721 | ccccggccgc caacccctga aagcagaacc tggcctccct ggccacagca gccttaccca |
| 3781 | ccgctctacg tgtcccgggc acttcccgca gccttcccgt ccctttctca tcggccttgt |
| 3841 | agttgtacag tgctgttggt ttgaaaaggt gatgtgtggg gagtgcggct catcactgag |
| 3901 | tagagaggta gaatttctat ttaaccagac ctgtagtagt attaccaatc cagttcaatt |
| 3961 | aaggtgattt tttgtaatta ttattatttt ggtgggacaa tctttaattt tctaaagata |
| 4021 | gcactaacat cagctcatta gccacctgtg cctgtccccg ccttggcccg gctggatgaa |
| 4081 | gcggcttccc cgcagggccc ccacttccca gtggctgctt cctggggacc cagggcaccc |
| 4141 | cggcaccttc aggcacgctc ctcagctggt cacctcccgg ctttgccgtt cagatggggc |
| 4201 | tcctgaggct caggagtgaa gatgccacag agccgggctc ccctaggctg cgtcgggcat |
| 4261 | gcttggaagc tggcctgcca ggaccttcca ccctggggcc tgtgtcagcc gccggccctc |
| 4321 | cgcaccctgg aagcacacgg cctctgggaa ggacagccct gaccttcggt tttccgagca |
| 4381 | cggtgtttcc caagaattct gggctggcgg cctggtggca gtgctggaga tgaccccgag |
| 4441 | cccctccccg tggggcaccc aggagggccc tgccggaatg tgcagcctgt gggtagtcgg |
| 4501 | ctggtgtccc tgtcgtggag ctggggtgcg tgatctggtg ctcgtccacg caggtgtgtg |
| 4561 | gtgtaaacat gtatgtgctg tacagagaga cgcgtgtgga gagagccgca caccagcgcc |
| 4621 | acccaggaaa ggcggagcgg ttaccagtgt tttgtgttta tttttaatca agacgtttcc |
| 4681 | cctgttttcc tataaatttg cttcgtgtaa gcaagtacat aaggaccctc ctttggtgaa |
| 4741 | atccgggttc gaatgaatat ctcaaggcag gagatgcatc tattttaaga tgctttggag |
| 4801 | cagacagctt tagccgttcc caatccttag caatgcctta gctgggacgc atagctaata |
| 4861 | ctttagagag gatgacagat ccataaagag agtaaagata agagaaaatg tctaaagcat |
| 4921 | ctggaaaggt aaaaaaaaaa aatctatttt tgtacaaatg taattttatc cctcatgtat |
| 4981 | acttggatat ggcgggggga gggctgggac tgtttcgttt ctgcttctag agattgaggt |
| 5041 | gaaagcttcg tccgagaaac gccaggacag acgatggcag aggagagggc tcctgtgacg |
| 5101 | gcggcgaggc ttgggaggaa accgccgcaa tgggggtgtc ttccctcggg gcaggagggt |
| 5161 | gggcctgagg ctttcaaggg ttttcttccc tttcgagtaa tttttaaagc cttgctctgt |
| 5221 | tgtgtcctgt tgccggctct ggccttcctg tgactgactg tgaagtggct tctccgtacg |
| 5281 | attgtctctg aaacatcgtg gcctcaggtg ccagggtttg atggacagta gcattagaat |
| 5341 | tgtggaaaag gaacacgcaa agggagaagt gtgagaggag aaacaaaata tgagcgttta |
| 5401 | aaatacatcg ccattcagtt cgttaaaaaa aaaaaaaaaa aaaaaaaaa |

| | |
|---|---|
| 71. | SEQ ID NO: 71 RXRα DBD nucleic acid sequence |

| | |
|---|---|
| | ac caagcacatc tgcgccatct |
| 481 | gcggggaccg ctcctcaggc aagcactatg gagtgtacag ctgcgagggg tgcaagggct |
| 541 | tcttcaagcg gacggtgcgc aaggacctga cctacacctg ccgcgacaac aaggactgcc |
| 601 | tgattgacaa gcggcagcgg aaccggtgcc agtactgccg ctaccagaag tgcctggcca |
| 661 | tgggcatg |

| | |
|---|---|
| 72. | SEQ ID NO: 72 RXRα hinge domain nucleic acid sequence |

| | |
|---|---|
| | aa gcgggaagcc gtgcaggagg agcggcagcg tggcaaggac cggaacgaga |
| 721 | atgaggtgga gtcgaccagc agcgccaacg aggacatgcc ggtggagagg atcctggagg |
| 781 | ctgagctggc cgtggagccc aagaccgaga cctacgtgga ggcaaacatg gggctgaacc |
| 841 | ccagc |

| | |
|---|---|
| 73. | SEQ ID NO: 73 Retinoic Acid Receptor α (RARα) amino acid sequence |

MASNSSSCPTPGGGHLNGYPVPPYAFFFPPMLGGLSPPGALTTLQHQLPVSGYSTPS
PATIETQSSSSEEIVPSPPSPPPLPRIYKPCFVCQDKSSGYHYGVSACEGCKGFFRRSIQ
KNMVYTCHRDKNCIINKVTRNRCQYCRLQKCFEVGMSKESVRNDRNKKKKEVPK
PECSESYTLTPEVGELIEKVRKAHQETFPALCQLGKYTTNNSSEQRVSLDIDLWDKF
SELSTKCIIKTVEFAKQLPGFTTLTIADQITLLKAACLDILILRICTRYTPEQDTMTFSD
GLTLNRTQMHNAGFGPLTDLVFAFANQLLPLEMDDAETGLLSAICLICGDRQDLEQP
DRVDMLQEPLLEALKVYVRKRRPSRPHMFPKMLMLKITDLRSISAKGAERVITLKME
IPGSMPPLIQEMLENSEGLDTLSGQPGGGGRDGGGLAPPPGSCSPSLSPSSNRSSPAT
HSP

| | |
|---|---|
| 74. | SEQ ID NO: 74 RARα nucleic acid sequence |

| | |
|---|---|
| 1 | gccatctggg cccaggcccc atgccccgag gaggggtggt ctgaagccca ccagagcccc |
| 61 | ctgccagact gtctgcctcc cttctgactg tggccgcttg gcatggccag caacagcagc |
| 121 | tcctgcccga cacctggggg cgggcacctc aatgggtacc cggtgcctcc ctacgccttc |
| 181 | ttcttccccc ctatgctggg tggactctcc ccgccaggcg ctctgaccac tctccagcac |
| 241 | cagcttccag ttagtggata tagcaoacca tccccagcca ccattgagac ccagagcagc |
| 301 | agttctgaag agatagtgcc cagccctccc tcgccacccc ctctaccccg catctacaag |
| 361 | ccttgctttg tctgtcagga caagtcctca ggctaccact atggggtcag cgcctgtgag |
| 421 | ggctgcaagg gcttcttccg ccgcagcatc cagaagaaca tggtgtacac gtgtcaccgg |
| 481 | gacaagaact gcatcatcaa caaggtgacc cggaaccgct gccagtactg ccgactgcag |
| 541 | aagtgctttg aagtgggcat gtccaaggag tctgtgagaa acgaccgaaa caagaagaag |
| 601 | aaggaggtgc ccaagcccga gtgctctgag agctacacgc tgacgccgga ggtgggggag |
| 661 | ctcattgaga aggtgcgcaa agcgcaccag gaaaccttcc ctgccctctg ccagctgggc |
| 721 | aaatacacta cgaacaacag ctcagaacaa cgtgtctctc tggacattga cctctgggac |
| 781 | aagttcagtg aactctccac caagtgcatc attaagactg tggagttcgc caagcagctg |
| 841 | cccggcttca ccaccctcac catcgccgac cagatcaccc tcctcaaggc tgcctgcctg |
| 901 | gacatcctga tcctgcggat ctgcacgcgg tacacgcccg agcaggacac catgaccttc |
| 961 | tcggacgggc tgaccctgaa ccggacccag atgcacaacg ctggcttcgg ccccctcacc |
| 1021 | gacctggtct ttgccttcgc caaccagctg ctgcccctgg agatggatga tgcggagacg |
| 1081 | gggctgctca gcgccatctg cctcatctgc ggagaccgcc aggacctgga gcagccggac |
| 1141 | cgggtggaca tgctgcagga gccgctgctg gaggcgctaa aggtctacgt gcggaagcgg |
| 1201 | aggcccagcc gcccccacat gttccccaag atgctaatga agattactga cctgcgaagc |
| 1261 | atcagcgcca agggggctga gcgggtgatc acgctgaaga tggagatccc gggctccatg |
| 1321 | ccgcctctca tccaggaaat gttggagaac tcagagggcc tggacactct gagcggacag |
| 1381 | ccggggggtg gggggcggga cgggggtggc ctggcccccc cgccaggcag ctgtagcccc |
| 1441 | agcctcagcc ccagctccaa cagaagcagc ccggccaccc actccccgtg accgcccacg |
| 1501 | ccacatggac acagccctcg ccctccgccc cggcttttct ctgcctttct accgaccatg |
| 1561 | tgaccccgca ccagccctgc ccccacctgc cctcccgggc agtactgggg accttccctg |
| 1621 | ggggacgggg agggaggagg cagcgactcc ttggacagag gcctgggccc tcagtggact |
| 1681 | gcctgctccc acagcctggg ctgacgtcag aggccgaggc caggaactga gtgaggcccc |
| 1741 | tggtcctggg tctcaggatg ggtcctgggg gcctcgtgtt catcaagaca cccctctgcc |
| 1801 | cagctcacca catcttcatc accagcaaac gccaggactt ggctccccca tcctcagaac |
| 1861 | tcacaagcca ttgctcccca gctggggaac ctcaacctcc cccctgcctc ggttggtgac |
| 1921 | agagggggtg ggacaggggc ggggggttcc ccctgtacat accctgccat accaacccca |
| 1981 | ggtattaatt ctcgctggtt ttgtttttat tttaattttt ttgttttgat ttttttaata |
| 2041 | agaattttca ttttaagcac atttatactg aaggaatttg tgctgtgtat tggggggagc |
| 2101 | tggatccaga gctggagggg gtgggtccgg gggagggagt ggctcggaag gggcccccac |
| 2161 | tctcctttca tgtccctgtg ccccccagtt ctcctcctca gccttttcct cctcagtttt |
| 2221 | ctctttaaaa ctgtgaagta ctaactttcc aaggcctgcc ttcccctccc tcccactgga |
| 2281 | gaagccgcca gcccctttct ccctctgcct gaccactggg tgtggacggt gtggggcagc |
| 2341 | cctgaaagga caggctcctg gccttggcac ttgcctgcac ccaccatgag gcatggagca |
| 2401 | gggcagagca agggccccgg gacagagttt tcccagacct ggctcctcgg cagagctgcc |
| 2461 | tcccgtcagg gcccacatca tctaggctcc ccagccccca ctgtgaaggg gctggccagg |
| 2521 | ggcccgagct gcccccaccc ccggcctcag ccaccagcac ccccataggg cccccagaca |
| 2581 | ccacacacat gcgcgtgcgc acacacacaa acacacacac actggacagt agatgggccg |
| 2641 | acacacactt ggcccgagtt cctccatttc cctggcctgc cccccacccc caacctgtcc |
| 2701 | cacccccgtg ccccctcctt accccgcagg acgggcctac aggggggtct cccctcaccc |
| 2761 | ctgcaccccc agctggggga gctggctctg ccccgacctc cttcaccagg ggttggggcc |
| 2821 | ccttcccctg gagcccgtgg gtgcacctgt tactgttggg ctttccactg agatctactg |
| 2881 | gataaagaat aaagttctat ttattct |

| | |
|---|---|
| 75. | SEQ ID NO: 75 RARα DBD nucleic acid sequence |

| | |
|---|---|
| | atctacaag |
| 361 | ccttgctttg tctgtcagga caagtcctca ggctaccact atggggtcag cgcctgtgag |
| 421 | ggctgcaagg gcttcttccg ccgcagcatc cagaagaaca tggtgtacac gtgtcaccgg |
| 481 | gacaagaact gcatcatcaa caaggtgacc cggaaccgct gccagtactg ccgactgcag |
| 541 | aagtgctttg aagtgggcat gtccaag |

| | |
|---|---|
| 76. | SEQ ID NO: 76 RARα hinge domain nucleic acid sequence |

| | |
|---|---|
| | gag tctgtgagaa acgaccgaaa caagaagaag |
| 601 | aaggaggtgc ccaagcccga gtgctctgag agctacacgc tgacgccgga ggtgggggag |
| 661 | ctcattgaga aggtgcgcaa agcgcaccag gaaaccttcc ctgccctctg ccagctgggc |
| 721 | aaatacacta cgaacaacag ctcagaacaa cgtgtctctc tggacattga cctc |

| | |
|---|---|
| 77. | SEQ ID NO: 77 Vitamin D Receptor (VDR) amino acid sequence |

MEAMAASTSLPDPGDFDRNVPRICGVCGDRATGFHFNAMTCEGCKGFFRRSMKRK
ALFTCPFNGDCRITKDNRRHCQACRLKRCVDIGMMKEFILTDEEVQRKREMILKRK
EEEALKDSLRPKLSEEQQRIIAILLDAHHKTYDPTYSDFCQFRPPVRVNDGGGSHPSR
PNSRHTPSFSGDSSSSCSDHCITSSDMMDSSSFSNLDLSEEDSDDPSVTLELSQLSMLP
HLADLVSYSIQKVIGFAKMIPGFRDLTSEDQIVLLKSSAIEVIMLRSNESFTMDDMSW
TCGNQDYKYRVSDVTKAGHSLELIEPLIKFQVGLKKLNLHEEEHVLLMAICIVSPDR
PGVQDAALIEAIQDRLSNTLQTYIRCRHPPPGSHLLYAKMIQKLADLRSLNEEHSKQ
YRCLSFQPECSMKLTPLVLEVFGNEIS

| | |
|---|---|
| 78. | SEQ ID NO: 78 VDR nucleic acid sequence |

| | |
|---|---|
| 1 | atggaggcaa tggcggccag cacttccctg cctgaccctg gagactttga ccggaacgtg |
| 61 | ccccggatct gtggggtgtg tggagaccga gccactggct ttcacttcaa tgctatgacc |
| 121 | tgtgaaggct gcaaaggctt cttcaggcga agcatgaagc ggaaggcact attcacctgc |
| 181 | cccttcaacg gggactgccg catcaccaag gacaaccgac gccactgcca ggcctgccgg |
| 241 | ctcaaacgct gtgtggacat cggcatgatg aaggagttca ttctgacaga tgaggaagtg |
| 301 | cagaggaagc gggagatgat cctgaagcgg aaggaggagg aggccttgaa ggacagtctg |
| 361 | cggcccaagc tgtctgagga gcagcagcgc atcattgcca tactgctgga cgcccaccat |
| 421 | aagacctacg accccaccta ctccgacttc tgccagttcc ggcctccagt tcgtgtgaat |
| 481 | gatggtggag ggagccatcc ttccaggccc aactccagac acactcccag cttctctggg |
| 541 | gactcctcct cctcctgctc agatcactgt atcacctctt cagacatgat ggactcgtcc |
| 601 | agcttctcca atctggatct gagtgaagaa gattcagatg acccttctgt gaccctagag |
| 661 | ctgtcccagc tctccatgct gccccacctg gctgacctgg tcagttacag catccaaaag |
| 721 | gtcattggct ttgctaagat gataccagga ttcagagacc tcacctctga ggaccagatc |
| 781 | gtactgctga agtcaagtgc cattgaggtc atcatgttgc gctccaatga gtccttcacc |
| 841 | atggacgaca tgtcctggac ctgtggcaac caagactaca agtaccgcgt cagtgacgtg |
| 901 | accaaagccg gacacagcct ggagctgatt gagcccctca tcaagttcca ggtgggactg |
| 961 | aagaagctga acttgcatga ggaggagcat gtcctgctca tggccatctg catcgtctcc |
| 1021 | ccagatcgtc ctggggtgca ggacgccgcg ctgattgagg ccatccagga ccgcctgtcc |
| 1081 | aacacactgc agacgtacat ccgctgccgc cacccgcccc cgggcagcca cctgctctat |
| 1141 | gccaagatga tccagaagct agccgacctg cgcagcctca atgaggagca ctccaagcag |
| 1201 | taccgctgcc tctccttcca gcctgagtgc agcatgaagc taacgcccct tgtgctcgaa |
| 1261 | gtgtttggca atgagatctc ctga |

| | |
|---|---|
| 79. | SEQ ID NO: 79 VDR DBD nucleic acid sequence |

| | |
|---|---|
| | cggatct gtggggtgtg Iggagaccga gccactggct ttcacttcaa tgctatgacc |
| 121 | tgtgaaggct gcaaaggctt cttcaggcga agcatgaagc ggaaggcact attcacctgc |
| 181 | cccttcaacg gggactgccg catcaccaag gacaaccgac gccactgcca ggcctgccgg |
| 241 | ctcaaacgct gtgtggacat cggcatgatg aag |

| | |
|---|---|
| 80. | SEQ ID NO: 80 VDR hinge domain nucleic acid sequence |

| | |
|---|---|
| | gagttca ttctgacaga tgaggaagtg |
| 301 | cagaggaagc gggagatgat cctgaagcgg aaggaggagg aggccttgaa ggacagtctg |
| 361 | cggcccaagc tgtctgagga gcagcagcgc atcattgcca tactgctgga cgcccaccat |
| 421 | aagacctacg accccaccta ctccgacttc tgccagttcc ggcctcca |

| | |
|---|---|
| 81. | SEQ ID NO: 81 ERα Hinge Domain Amino Acid Sequence |

MKGGVRKDRRGGRMLKHKRQRDDGEGRGEVGSAGDMRAANLWPSPLMIKRS

| | |
|---|---|
| 82. | SEQ ID NO: 82 c-Myb DNA binding Domain Amino Acid Sequence |

MARRPRHSIYSSDEDDEDFEMCDHDYDGLLPKSGKRHLGKTRWTREE

| | |
|---|---|
| 83. | SEQ ID NO: 83 Hinge Domain Nucleic Acid Sequence |

CTCTGGATCCGGTGGAGGTGGTTCTGGAGGAGGTGGTTCCGGAGGTGGAGGAA
AGGAGACGCGTTACGCTG

| | |
|---|---|
| 84. | SEQ ID NO: 84 Hinge Domain Amino Acid Sequence |

LWIRWRWFWRRWFRRWRKGDALTL

1. A composition comprising the formula W-Z-X, wherein W comprises a first DNA binding domain, X comprises a second DNA binding domain and Z comprises a hinge domain, and wherein the composition binds a DNA binding site.

2. The composition of claim 1, wherein the composition binds a hormone response element selected from the group of response elements consisting of Estrogen Response Element (ERE), Glucocorticoid Response Element (GRE), Androgen Response Element (ARE), Progesterone Response Element (PRE), Thyroid Response Element (TRE), Retinoid X Response Element (RXRE), all-trans Retinoic Acid Response Elements (RARE), and Vitamin D Response Element (VDRE).

3. The composition of claim 1, further comprising Y, an activation domain or a repressor domain.

4. The hinge domain of claim 1, wherein the hinge domain comprises an ER D-domain.

5. The hinge domain of claim 4, wherein the ER D-domain comprises the ER D-domain from ERα or ERβ.

6. (canceled)

7. The composition of claim 1, wherein the first DNA binding domain comprises a DNA binding domain of a transcription factor.

8. The composition of claim 7, wherein the transcription factor can be selected from the group of transcription factors consisting of Estrogen receptor (ER), Glucocorticoid receptor (GR), Androgen receptor (AR), Progesterone receptor (PR), Thyroid receptor (TR), Retinoid X receptor (RXR), all-trans Retinoic Acid receptor (RAR), and Vitamin D receptor (VDR)

9. The composition of claim 7, wherein the transcription factor ER comprises ERα or ERβ.

10. (canceled)

11. The composition of claim 1, wherein the second DNA binding domain comprises a DNA binding domain of a transcription factor.

12. The composition of claim 11, wherein the transcription factor can be selected from the group of transcription factors consisting of Estrogen receptor (ER), Glucocorticoid receptor (GR), Androgen receptor (AR), Progesterone receptor (PR), Thyroid receptor (TR), Retinoid X receptor (RXR), all-trans Retinoic Acid receptor (RAR), and Vitamin D receptor (VDR).

13. The composition of claim 11, wherein the transcription factor ER comprises ERα or ERβ.

14. (canceled)

15. The composition of claim 1, wherein the DNA binding site comprises a first half-site and a second half-site.

16. The composition of claim 15, wherein the first half-site comprises the sequence of SEQ ID NO: 1.

17. The composition of claim 15, wherein the first half-site comprises the sequence of SEQ ID NO: 2.

18. The composition of claim 15, wherein the second half-site comprises the sequence of SEQ ID NO: 1.

19. The composition of claim 15, wherein the second half-site comprises the sequence of SEQ ID NO: 2.

20. The composition of claim 15, wherein the first half-site and the second half-site are direct repeats.

21. The composition of claim 15, wherein the first half-site and the second half-site are inverted repeats.

22. The composition of claim 15, wherein the first half-site and the second half site are not separated by a nucleotide.

23. The composition of claim 15, wherein the first half-site and the second half site are separated by a 1, 2, 3, 4, 5, 10, or 15 nucleotide spacer.

24-29. (canceled)

30. The composition of claim 1, wherein the binding site comprises the sequence as set forth in SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

31-49. (canceled)

50. The composition of claim 1, wherein the composition comprises an activation domain.

51. The composition of claim 50, wherein the activation domain comprises one, two, three, or four VP16 domain(s).

52-54. (canceled)

55. The composition of claim 50, wherein the activation domain comprises one, two, or four p65.

56-57. (canceled)

58. The composition of claim 50, wherein the activation domain comprises a VP16 and a p65.

59. The composition of claim 50, wherein the activation domain comprises two VP16 and two p65.

60. The composition of claim 1, wherein the composition comprises a repressor domain.

61. The composition of claim 60, wherein the repressor domain comprises one, two, or four Krüpple associated box(es) (KRAB).

62-63. (canceled)

64. The composition of claim 60, wherein the repressor domain comprises one, two, or four Sin3 interaction domain(s) (SID).

65-66. (canceled)

67. The composition of claim 60, wherein the repressor domain comprises a KRAB and a SID.

68. The composition of claim 60, wherein the repressor domain comprises two KRAB and two SID.

69. A nucleic acid encoding the composition of claim 1.

70. A vector comprising the nucleic acid of claim 69.

71. A cell comprising the vector of claim 70.

72. An animal comprising the cell of claim 71.

73. A cell comprising the nucleic acid of claim 69.

74. An animal comprising the cell of claim 73.

75. A method of identifying a gene that is under transcriptional control of a hormone response element comprising contacting a cell containing the hormone response element with the composition of claim 1 and monitoring the cell for changes in the transcription.

76. A method of identifying a gene that is under transcriptional control of a hormone response element comprising contacting a cell containing the hormone response element with the composition of claim 51 and monitoring the cell for changes in the transcription.

77. A method of identifying a gene that is under transcriptional control of a hormone response element comprising contacting a cell containing the hormone response element with the composition of claim 60 and monitoring the cell for changes in the transcription.

78. A method of treating cancer in a subject comprising administering to the subject the composition of claim 1.

79. A method of treating cancer in a subject comprising administering to the subject the composition of claim 60.

80. A method of inhibiting the transcription of a gene comprising contacting a cell containing the gene with the composition of claim 60.

81. The method of claim 80, wherein the cell is in a subject.

82. The method of claim 81, wherein the subject has cancer.

83. A method of overexpressing a gene in a cell comprising contacting the gene with the composition of claim 51.

84. A method of treating cancer in a subject comprising administering to the subject the composition of claim 1 wherein the composition causes the destruction of cancerous cells through the overexpression of genes under the control of hormone response elements.

85. A method of treating cancer in a subject comprising administering to the subject the composition of claim 51 so as to cause the destruction of cancerous cells through the overexpression of genes under the control of hormone response elements.

86. The composition of claim 1, wherein the first DNA binding domain comprises a sequence having at least 80% identity to the sequence set forth in SEQ ID NOs; 23, 25, 27, 29, 31, 33, 35, 37, or 39.

87. (canceled)

88. The composition of claim 1, wherein the second DNA binding domain comprises a sequence having at least 80% identity to the sequence set forth in SEQ ID NOs; 23, 25, 27, 29, 31, 33, 35, 37, or 39.

89. (canceled)

90. The composition of claim 87, wherein the second DNA binding domain comprises a sequence having at least 80% identity to the sequence set forth in SEQ ID NOs; 23, 25, 27, 29, 31, 33, 35, 37, or 39.

91. (canceled)

92. The composition of claim 1, wherein the hinge domain comprises a sequence having at least 80% identity to the sequence set forth in SEQ ID NOs; 24, 26, 28, 30, 32, 34, 36, 38, or 40.

93. (canceled)

94. The composition of claim 90, wherein the hinge domain comprises a sequence having at least 80% identity to the sequence set forth in SEQ ID NOs; 24, 26, 28, 30, 32, 34, 36, 38, or 40.

95. (canceled)
